(19) United States
(12) Patent Application Publication
(10) Pub. No.: US 2016/0115447 A1
(43) Pub. Date: Apr. 28, 2016

(54) COMPOSITIONS AND METHODS FOR IMPROVING INDUCED NEURON GENERATION

(71) Applicant: PRESIDENT AND FELLOWS OF HARVARD COLLEGE, Cambridge, MA (US)

(72) Inventors: Andre BLUMENSTEIN, Cambridge, MA (US); Justin ICHIDA, Boston, MA (US); Kevin C. EGGAN, Boston, MA (US); Lee L. RUBIN, Wellesley, MA (US)

(21) Appl. No.: 14/898,028

(22) PCT Filed: Jun. 11, 2014
(86) PCT No.: PCT/US14/41939

§ 371 Date: Dec. 11, 2015

Related U.S. Application Data
(60) Provisional application No. 61/833,911, filed on Jun. 11, 2013.

(51) Int. Cl. C12N 5/0793; A61K 35/30

(52) U.S. Cl. CPC C12N 5/0619; A61K 35/30; C12N 2501/16; C12N 2501/727

(57) ABSTRACT

The present inventions relate to methods and compositions useful for improving the efficiency of inducing the generation of neurons from non-neuronal cell types, for example, by contacting the cell or cell culture medium with one or more agents which inhibit Activin and/or PLK1 signaling. Also disclosed are methods for promoting neuron survival, for example, by inhibiting Activin and/or PLK1 signaling, and methods for promoting the survival of intermediates in a cell differentiation pathway, for example, by inhibiting Activin and/or PLK1 signaling.

### RELATED APPLICATIONS

This application claims the benefit of U.S. Provisional Application No. 61/833,911, filed Jun. 11, 2013, the entire teachings of which are incorporated herein by reference.

### BACKGROUND OF THE INVENTION

The mammalian nervous system comprises many distinct neuronal subtypes, each with its own phenotype and differential sensitivity to degenerative disease. Although specific neuronal types can be isolated from rodents or engineered from stem cells for translational studies, transcription factor-mediated reprogramming provides a more direct route to their generation. Recent studies have demonstrated that the forced expression of select transcription factors is sufficient to convert mouse and human fibroblasts and stem cells directly into a variety of neuronal subtypes. However, the utility of this approach is currently limited by the low efficiency of conversion. Accordingly, there exists a need for agents that are able to increase the efficiency of induced neuron generation.

### SUMMARY OF THE INVENTION

In some aspects, the disclosure provides methods and compositions for improving the efficiency of inducing the generation of neurons (e.g., motor neurons) from non-neuronal cell types (e.g., from a less differentiated cell such as a stem cell or pluripotent cell or from an alternate cell type such as a non-neuronal somatic cell). In some aspects the methods comprise inhibiting Activin signaling, inhibiting Polo-like kinase I (PLK1) signaling, or inhibiting both Activin signaling and PLK1 signaling. The disclosure also provides methods for promoting neuron (e.g., motor neuron) survival, for example, by inhibiting Activin signaling, and methods for promoting the survival of intermediates in a cell differentiation pathway, for example, by inhibiting PLK1 signaling. In certain aspects inhibition of Activin signaling or of the Activin signaling pathway comprises decreasing the level or activity of one or more of activin-like kinase 4 (ALK4), activin-like kinase 5 (ALK5), or activin-like kinase 7 (ALK7). In certain aspects inhibition of PLK1 signaling or of the PLK1 signaling pathway comprises decreasing the level or activity of PLK1.

In some aspects, the disclosure provides methods for improving the efficiency of neuron generation or production (e.g., motor neuron generation or production) from a somatic cell, comprising inhibiting Activin signaling (e.g., by decreasing the level or activity of one or more of ALK4, ALK5, and ALK7) in the cell, thereby increasing the efficiency or rate of motor neuron formation. In some aspects the neuron is generated from the somatic cell via factor-mediated transdifferentiation. In some aspects the efficiency or rate of neuron formation is increased at least 2-fold, at least 5-fold, at least 10-fold, at least 20-fold, etc. compared to the efficiency or rate of neuron formation when Activin signaling is not inhibited. In some aspects inhibiting Activin signaling comprises contacting the cell or cell culture medium with one or more agents which inhibit Activin signaling. In some aspects the agent which inhibits Activin signaling inhibits Activin. In some aspects the agent which inhibits Activin signaling inhibits one or more of ALK4, ALK5 and ALK7. In some aspects the resulting neuron exhibits at least two characteristics of a functional neuron (e.g., of a functional motor neuron).

In some aspects, the disclosure provides methods for improving the efficiency of neuron generation or production (e.g., motor neuron generation or production) from a somatic cell, comprising inhibiting PLK1 signaling in the cell, thereby increasing the efficiency or rate of motor neuron formation. In some aspects the neuron is generated from the somatic cell via factor-mediated transdifferentiation. In some aspects the efficiency or rate of neuron formation is increased at least 2-fold, at least 5-fold, at least 10-fold, at least 20-fold, etc. compared to the efficiency or rate of neuron formation when PLK1 signaling is not inhibited. In some aspects inhibiting PLK1 signaling comprises contacting the cell or cell culture medium with one or more agents which inhibit PLK1 signaling. In some aspects the agent which inhibits PLK1 signaling inhibits PLK1. In some aspects the resulting neuron exhibits at least two characteristics of a functional neuron (e.g., of a functional motor neuron).

In some aspects, the disclosure provides methods for improving the efficiency of neuron generation or production (e.g., motor neuron generation or production) from a somatic cell, comprising inhibiting both Activin signaling and PLK1 signaling in the cell, thereby increasing the efficiency or rate of motor neuron formation. In some aspects the neuron is generated from the somatic cell via factor-mediated transdifferentiation. In some aspects the efficiency or rate of neuron formation is increased at least 2-fold, at least 5-fold, at least 10-fold, at least 20-fold, at least 25-fold, at least 50-fold, etc. compared to the efficiency or rate of neuron formation when either or both of Activin signaling and PLK1 signaling are not inhibited. In some aspects inhibiting Activin signaling and PLK1 signaling comprises contacting the cell or cell culture medium with one or more agents which inhibit Activin signaling and one or more agents which inhibit PLK1 signaling. In some aspects the gent which inhibits Activin signaling inhibits Activin. In some aspects the agent which inhibits Activin signaling inhibits one or more of ALK4, ALK5 and ALK7. In some aspects the agent which inhibits PLK1 signaling inhibits PLK1. In some aspects the resulting neuron exhibits at least two characteristics of a functional neuron (e.g., of a functional motor neuron).

In some aspects, the disclosure provides methods for improving the efficiency of neuron generation or production (e.g., motor neuron generation or production) from a less differentiated cell, comprising inhibiting Activin signaling (e.g., by decreasing the level or activity of one or more of ALK4, ALK5, and ALK7) in the cell, thereby increasing the efficiency or rate of motor neuron formation. In some aspects the neuron is generated from the less differentiated cell via factor-mediated differentiation. In some aspects the efficiency or rate of neuron formation is increased at least 2-fold, at least 5-fold, at least 10-fold, at least 20-fold, etc. compared to the efficiency or rate of neuron formation when Activin signaling is not inhibited. In some aspects inhibiting Activin signaling comprises contacting the cell or cell culture medium with one or more agents which inhibit Activin signaling. In some aspects the agent which inhibits Activin signaling inhibits Activin. In some aspects the agent which inhibits Activin signaling inhibits one or more of ALK4, ALK5 and ALK7. In some aspects the resulting neuron exhibits at least two characteristics of a functional neuron (e.g., of a functional motor neuron).

In some aspects, the disclosure provides methods for improving the efficiency of neuron generation or production (e.g., motor neuron generation or production) from a less differentiated cell, comprising inhibiting PLK1 signaling in the cell, thereby increasing the efficiency or rate of motor neuron formation. In some aspects the neuron is generated from the less differentiated cell via factor-mediated differentiation. In some aspects the efficiency or rate of neuron formation is increased at least 2-fold, at least 5-fold, at least 10-fold, at least 20-fold, etc. compared to the efficiency or rate of neuron formation when PLK1 signaling is not inhibited. In some aspects inhibiting PLK1 signaling comprises contacting the cell or cell culture medium with one or more agents which inhibit PLK1 signaling. In some aspects the agent which inhibits PLK1 signaling inhibits PLK1. In some aspects the resulting neuron exhibits at least two characteristics of a functional neuron (e.g., of a functional motor neuron).

In some aspects, the disclosure provides methods for improving the efficiency of neuron generation or production (e.g., motor neuron generation or production) from less differentiated cell, comprising inhibiting both Activin signaling and PLK1 signaling in the cell, thereby increasing the efficiency or rate of motor neuron formation. In some aspects the neuron is generated from the less differentiated cell via factor-mediated differentiation. In some aspects the efficiency or rate of neuron formation is increased at least 2-fold, at least 5-fold, at least 10-fold, at least 20-fold, at least 25-fold, at least 50-fold, etc. compared to the efficiency or rate of neuron formation when either or both of Activin signaling and PLK1 signaling are not inhibited. In some aspects inhibiting Activin signaling and PLK1 signaling comprises contacting the cell or cell culture medium with one or more agents which inhibit Activin signaling and one or more agents which inhibit PLK1 signaling. In some aspects the agent which inhibits Activin signaling inhibits Activin. In some aspects the agent which inhibits Activin signaling inhibits one or more of ALK4, ALK5 and ALK7. In some aspects the agent which inhibits PLK1 signaling inhibits PLK1. In some aspects the resulting neuron exhibits at least two characteristics of a functional neuron (e.g., of a functional motor neuron).

In some embodiments of any aspect described herein, the somatic cell is a fibroblast. In some embodiments of any aspect described herein the cell is a mouse cell. In some embodiments of any aspect described herein, the cell is a human cell, such as, for example, a patient-derived cell.

In some embodiments of any aspect described herein, a characteristic of the functional motor neuron is expression of at least two motor neuron specific genes selected from the group consisting of: β2-tubulins, Map2, synapsins, synaptophysin, synaptotagmins, NeuroD, Isl1, cholineacetyltransferase (ChAT). In some embodiments of any aspect described herein, the β2-tubulin is selected from Tubb2a and Tubb2b. In some embodiments of any aspect described herein, the synapsins are selected from Syn1 and Syn2. In some embodiments of any aspect described herein, the synaptotagmins are selected from: Syt1, Syt4, Syt13, Syt 16. In some embodiments of any aspect described herein, the ChAT is vesicular ChAT. In some embodiments of any aspect described herein, a characteristic of the functional motor neuron is expression of a decreased level of a fibroblast gene, such as a gene selected from the group consisting of: Snail 1, thy1 and Fsp1, by a statistically significant level as compared to the somatic cell from which the motor neuron was derived. In some embodiments of any aspect described herein, a characteristic of the functional motor neuron is a motor neuron morphology comprising a cell body with axonal projections which form functional synaptic junctions with muscle cells. In some embodiments of any aspect described herein, a characteristic of the functional motor neuron is an average resting potential of below −50 mV. In some embodiments of any aspect described herein, the motor neuron has an average resting potential of between −65 mV and −50 mV. In some embodiments of any aspect described herein, a characteristic of the functional motor neuron is a functional characteristic selected from the group consisting of: ability to fire action potentials, produce an outward current in response to glycine, GABA or kainate, or produce an inward current in response to glutamate.

In some embodiments of any aspect described herein, the level or activity of ALK4, ALK5, and/or ALK7 is inhibited by contacting the cell with an agent which decreases the level or activity of ALK4, ALK5, and ALK7. In some embodiments of any aspect described herein, the agent is selected from the group consisting of small organic or inorganic molecules; saccharines; oligosaccharides; polysaccharides; a biological macromolecule selected from the group consisting of antibodies, peptides, proteins, peptide analogs and derivatives, and dominant negative variants; peptidomimetics; nucleic acids selected from the group consisting of microRNAs, siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof. In some embodiments of any aspect described herein, the agent is RepSox or an analog or derivative thereof. In some embodiments of any aspect described herein, the contacting is done during at least one time period from days 1 to 5, days 6 to 10, and days 11 to 5 of the differentiation process.

In some embodiments of any aspect described herein, the level or activity of PLK1 is inhibited by contacting the cell with an agent which decreases the level or activity of PLK1. In some embodiments of any aspect described herein, the agent is selected from the group consisting of small organic or inorganic molecules; saccharines; oligosaccharides; polysaccharides; a biological macromolecule selected from the group consisting of antibodies, peptides, proteins, peptide analogs and derivatives, and dominant negative variants; peptidomimetics; nucleic acids selected from the group consisting of microRNAs, siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof. In some embodiments of any aspect described herein, the agent is BI 2536 or an analog or derivative thereof. In some embodiments of any aspect described herein, the contacting is done during the time period from days 6 to 10 of the differentiation process.

In some embodiments of any aspect described herein, the method is an in vitro method. In some embodiments of any aspect described herein, the method is an ex vivo method. In some embodiments of any aspect described herein, the cell is a mammalian cell. In some embodiments of any aspect described herein, the cell is obtained from a subject, e.g., a human subject. In some embodiments of any aspect described herein, the subject has, or is at risk of developing, a disease or disorder which causes or results from actual or functional neuronal deficiency. In some embodiments of any aspect described herein, the disease or disorder is selected from the group consisting of amyotrophic lateral sclerosis (ALS) or spinal muscular atrophy (SMA) or a disease, condition, or symptom associated therewith.

In some embodiments of any aspect described herein, the neuron is a motor neuron or a motor neuron-like cell. In some embodiments of any aspect described herein, the neuron is a spinal motor neuron. In some embodiments of any aspect described herein, the neuron is a Hb9::GFP+ spinal motor neuron.

In some aspects, the disclosure provides an isolated population of neurons obtained from a population of somatic cells by a process of transdifferentiation and inhibition of Activin signaling in the population of cells. In some aspects, the disclosure provides an isolated population of neurons obtained from a population of somatic cells by a process of transdifferentiation and inhibition of PLK1 signaling in the population of cells. In some aspects, the disclosure provides an isolated population of neurons obtained from a population of somatic cells by a process of transdifferentiation and inhibition of both Activin signaling and PLK1 signaling in the population of cells.

In some aspects, the disclosure provides an isolated population of neurons obtained from a population of less differentiated cells by a process of differentiation and inhibition of Activin signaling in the population of cells. In some aspects, the disclosure provides an isolated population of neurons obtained from a population of less differentiated cells by a process of differentiation and inhibition of PLK1 signaling in the population of cells. In some aspects, the disclosure provides an isolated population of neurons obtained from a population of less differentiated cells by a process of differentiation and inhibition of both Activin signaling and PLK1 signaling in the population of cells.

In some aspects, the disclosure provides an isolated population of neurons obtained or prepared according to any of the methods described herein.

In some aspects, the disclosure contemplates the use of an isolated population of neurons described herein for administering to a subject in need thereof.

In some aspects, the disclosure provides a kit comprising: (a) an agent or composition which inhibits Activin signaling (e.g., which inhibits the level or activity of ALK4, ALK5, and ALK7); and (b) an agent or composition which inhibits PLK1 signaling (e.g., which inhibits the level or activity of PLK1).

In some embodiments of any aspect described herein, the kit further comprises at least one cell (e.g., a somatic cell, a less differentiated cell, etc.). In some embodiments of any aspect described herein, the kit further comprising instructions for differentiation of the cell into a neuron (e.g., exhibiting at least two characteristics of a functional neuron).

In some aspects, the disclosure provides a composition comprising at least one cell and at least one agent which inhibits Activin signaling. In some aspects, the disclosure provides a composition comprising at least one cell and at least one agent which inhibits PLK1 signaling. In some aspects, the disclosure provides a composition comprising: (a) at least one cell; (b) at least one agent which inhibits Activin signaling; and (c) at least one agent which inhibits PLK1 signaling. In some embodiments of any aspect described herein, the composition further comprises one or more factors which facilitate differentiation from a less differentiated cell or transdifferentiation from a somatic cell.

In some aspects, the disclosure provides methods for increasing neuron survival (e.g., motor neuron survival), comprising inhibiting Activin signaling (e.g., by decreasing the level or activity of one or more of ALK4, ALK5, and ALK7) in the cell. In some embodiments the neuron is an isolated neuron. In some embodiments the neuron is generated from a somatic cell, e.g., via factor-mediated transdifferentiation. In some aspects the neuron is generated from a less differentiated cell, e.g., via factor-mediated differentiation. In some aspects inhibiting Activin signaling comprises contacting the cell or cell culture medium with one or more agents which inhibit Activin signaling. In some aspects the agent which inhibits Activin signaling inhibits Activin. In some aspects the agent which inhibits Activin signaling inhibits one or more of ALK4, ALK5 and ALK7. In some aspects the resulting neuron exhibits at least two characteristics of a functional neuron (e.g., of a functional motor neuron).

In some aspects, the disclosure provides methods for improving the survival of intermediates in a cell differentiation pathway (e.g., a neuron differentiation pathway), comprising inhibiting PLK1 signaling in the cell. In some embodiments the cell (e.g., a neuron) is generated from a somatic cell, e.g., via factor-mediated transdifferentiation. In some aspects the cell (e.g., a neuron) is generated from a less differentiated cell, e.g., via factor-mediated differentiation. In some aspects inhibiting PLK1 signaling comprises contacting the cell or cell culture medium with one or more agents which inhibit PLK1 signaling. In some aspects the agent which inhibits PLK1 signaling inhibits PLK1.

In some embodiments the disclosure relates to a method for improving the efficiency of neuron generation from a somatic cell, comprising (a) exposing the somatic cell to conditions sufficient for transdifferentiation of the somatic cell into a neuron; and (b) inhibiting one or both of Activin signaling and PLK1 signaling in the cell, thereby increasing the efficiency of neuron formation as compared with the efficiency when neither Activin signaling nor PLK1 signaling is inhibited. In some aspects the conditions sufficient for transdifferentiation of the somatic cell are conditions sufficient for factor-mediated transdifferentiation. In some embodiments the disclosure relates to a method for improving the efficiency of neuron generation from a less differentiated cell, comprising (a) exposing the less differentiated cell to conditions sufficient for differentiation of the less differentiated cell into a neuron; and (b) inhibiting one or both of Activin signaling and PLK1 signaling in the cell, thereby increasing the efficiency of neuron formation as compared with the efficiency when neither Activin signaling nor PLK1 signaling is inhibited. In some aspects the conditions sufficient for differentiation of the less differentiated cell are conditions sufficient for factor-mediated differentiation.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A and 1B illustrate the screens performed to identify small molecule enhancers of induced motor neuron (iMN) conversion. FIG. 1A is a schematic illustration depicting the primary screen for small molecules enhancers of iMN conversion via viral transduction with 7 transcription factors performed on fibroblasts harvested from 2 month old Hb9::GFP mice. FIG. 1B is a graphical illustration depicting the results of a secondary screen performed on the top hits identified by the primary screen depicted in FIG. 1A, pointing to two lead compounds as effective enhancers of iMN conversion.

FIGS. 2A and 2B are chemical structures of lead compounds identified in the screens shown in FIGS. 1A and 1B. FIG. 2A shows the chemical structure of RepSox, a TGF-beta, activin, and nodal inhibitor. FIG. 2B shows the chemical structure of BI 2536, a polo-like kinase I (PLK1) inhibitor

FIG. 3 is a bar graph demonstrating that combinations of small molecules identified in the screens result in a greater increase in efficiency than any compound individually, indicating that they act via divergent mechanisms.

FIG. 4 is a combined schematic illustration and bar graph showing that RepSox improved iMN conversion regardless of the time it is added to the culture medium, whereas BI 2536 improved conversion only during days 6-10.

FIGS. 5A and 5B are bar graphs illustrating that chemical treatment greatly promoted the survival of flow-purified mouse and human motor neurons in culture, indicating that Activin inhibition can act by promoting neuronal survival. FIGS. 5A and 5B are bar graphs demonstrating that RepSox promotes survival of FACS-sorted iMNs in wild-type (WT) and SOD1G93A motor neurons, respectively.

FIG. 6 is a line graph demonstrating that RepSox promotes survival of Hb9::GFP+ intermediates exhibiting a non-neuronal morphology.

FIG. 7 is a bar graph demonstrating that RepSox promotes generation of patient specific human iMNs.

FIG. 8 is a bar graph depicting the results of mechanistic studies of specific proteinaceous inhibitors of each RepSox signaling pathway, indicating that specifically inhibiting actavin signaling promotes hESC-derived motor neuron survival to a similar extent as RepSox, whereas inhibition of TGF-beta or Nodal signaling achieves only modest improvements in survival.

FIG. 9 is a bar graph demonstrating that RepSox enhances induced neuron (iN) conversion.

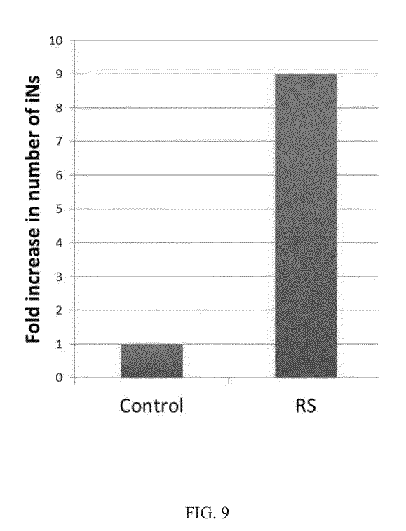

### DETAILED DESCRIPTION OF THE INVENTION

The disclosure relates to compositions, methods, kits, and agents for producing functional neurons (e.g., motor neurons) from non-neuronal cell types and populations of functional neurons produced by those compositions, methods, kits, and agents for use in screening, cell therapies and various methods of treatment.

The disclosure also relates to methods and compositions for improving the efficiency of inducing the generation of neurons (e.g., motor neurons) from non-neuronal cell types (e.g., from a less differentiated cell such as a stem cell or pluripotent cell or from an alternate cell type such as a non-neuronal somatic cell). The disclosure also provides methods for promoting neuron (e.g., motor neuron) survival, for example, by inhibiting Activin signaling, and methods for promoting the survival of intermediates in a cell differentiation pathway, for example, by inhibiting PLK1 signaling.

In certain aspects, the disclosure provides compositions, methods, kits, and agents for the direct conversion of non-neuronal cell types (e.g., somatic cells) to functional neurons (e.g., functional motor neurons (iMNs)), without the non-neuronal cell becoming an induced pluripotent stem cell (iPS) intermediate prior to being transdifferentiated into a functional neuron.

In certain aspects, the disclosure provides a population of induced neurons iNs (e.g., induced motor neurons iMNs) derived from a non-neuronal cell (e.g., somatic cell) and methods, compositions, kits, and agents for the direct reprogramming of cells, such as a somatic cell (e.g., fibroblast) to an iN.

In an aspect, the disclosure provides a method for converting (e.g., transdifferentiating) a non-neuronal cell (e.g., somatic cell) into a neuron (e.g., motor neuron) by inhibiting the level or activity of activin-like kinase 4 (ALK4), activin-like kinase 5 (ALK5), and activin-like kinase 7 (ALK7) in the non-neuronal cell.

In some embodiments, a method for converting a non-neuronal cell into a neuron comprises inhibiting the level or activity of activin-like kinase 4 (ALK4), activin-like kinase 5 (ALK5), and activin-like kinase 7 (ALK7) in the non-neuronal cell, thereby converting the non-neuronal cell into a neuron, wherein the neuron exhibits at least two characteristics of a functional neuron.

In some embodiments, a method for converting (e.g., transdifferentiating) a somatic cell into a motor neuron comprises inhibiting the level or activity of activin-like kinase 4 (ALK4), activin-like kinase 5 (ALK5), and activin-like kinase 7 (ALK7) in the somatic cell, thereby converting the somatic cell into a motor neuron, wherein the motor neuron exhibits at least two characteristics of a functional motor neuron.

In an aspect, the disclosure provides a method for improving the efficiency of inducing the generation of neurons (e.g., motor neurons) from non-neuronal cell types (e.g., from a less differentiated cell such as a stem cell or pluripotent cell or from an alternate cell type such as a non-neuronal somatic cell), comprising inhibiting the level or activity of ALK4, ALK5, and ALK7 in the non-neuronal cell, thereby increasing the efficiency or rate of neuron formation (e.g., motor neuron formation). In some embodiments, inhibiting the level or activity ALK4, ALK5, and ALK7 in the somatic cell increases the efficiency or rate of neuron formation by a factor of at least 2.5 fold, at least 2.6 fold, at least 2.7 fold, at least 2.8 fold, at least 2.9 fold, at least 3 fold, at least 3.3 fold, at least 3.6 fold, at least 3.8 fold, at least 4.1 fold, at least 4.4 fold, at least 4.7 fold, at least 4.8 fold, at least 5.0 fold, at least 5.1 fold, at least 5.4 fold, at least 5.6 fold, at least 5.9 fold, at least 6.0 fold, at least 6.2 fold, at least 6.4 fold, at least 6.5 fold, at least 6.7 fold, at least 6.9 fold, at least 7.0 fold, at least 7.2 fold, at least 7.4 fold, at least 7.7 fold, at least 7.9 fold, at least 8.2 fold, at least 8.5 fold, at least 9.0 fold, at least 9.1 fold, at least 9.2 fold, at least 9.3 fold, at least 9.4 fold, at least 9.5 fold or more compared to forced expression of transdifferentiating transcription factors. In some embodiments, inhibiting the level or activity ALK4, ALK5, and ALK7 in the non-neuronal cell increases the efficiency or rate of neuron formation by a factor of at least 10 fold or more compared to forced expression of transdifferentiating transcription factors.

In an aspect, the disclosure provides a method for improving the efficiency of motor neuron generation or production from a somatic cell, comprising inhibiting the level or activity of ALK4, ALK5, and ALK7 in the somatic cell, thereby increasing the efficiency or rate of motor neuron formation. In some embodiments, inhibiting the level or activity ALK4, ALK5, and ALK7 in the somatic cell increases the efficiency or rate of motor neuron formation via factor-mediated conversion of the somatic cell into a motor neuron by a factor of at least 2.5 fold, at least 2.6 fold, at least 2.7 fold, at least 2.8 fold, at least 2.9 fold, at least 3 fold, at least 3.3 fold, at least 3.6 fold, at least 3.8 fold, at least 4.1 fold, at least 4.4 fold, at least 4.7 fold, at least 4.8 fold, at least 5.0 fold, at least 5.1 fold, at least 5.4 fold, at least 5.6 fold, at least 5.9 fold, at least 6.0 fold, at least 6.2 fold, at least 6.4 fold, at least 6.5 fold, at least 6.7 fold, at least 6.9 fold, at least 7.0 fold, at least 7.2 fold, at least 7.4 fold, at least 7.7 fold, at least 7.9 fold, at least 8.2 fold, at least 8.5 fold, at least 9.0 fold, at least 9.1 fold, at least 9.2 fold, at least 9.3 fold, at least 9.4 fold, at least 9.5 fold or more. In some embodiments, inhibiting the level or activity ALK4, ALK5, and ALK7 in the somatic cell increases the rate or efficiency of motor neuron formation via factor-mediated conversion of the somatic cell into a motor neuron by a factor of at least 10 fold or more compared to forced expression of transdifferentiating transcription factors.

In an aspect, the disclosure provides a method for converting a non-neuronal cell (e.g., a somatic cell) into a neuron (e.g., motor neuron) by inhibiting the level or activity of PLK1 in the somatic cell. In some embodiments, a method for converting a non-neuronal cell into a neuron comprises inhibiting in the level or activity of PLK1 in the somatic cell, thereby converting the non-neuronal cell into a neuron, wherein the neuron exhibits at least two characteristics of a functional neuron.

In an aspect, the disclosure provides a method for converting (e.g., transdifferentiating) a somatic cell into a motor neuron by inhibiting the level or activity of PLK1 in the somatic cell. In some embodiments, a method for converting a somatic cell into a motor neuron comprises inhibiting in the level or activity of PLK1 in the somatic cell, thereby converting the somatic cell into a motor neuron, wherein the motor neuron exhibits at least two characteristics of a functional motor neuron.

In an aspect, a method for improving the efficiency of neuron generation or production (e.g., motor neuron generation or production) from a non-neuronal cell (e.g., a somatic cell) comprises inhibiting the level or activity of Polio-like kinase I (PLK1) in the non-neuronal cell (e.g., somatic cell), thereby increasing the rate or efficiency of neuron generation or production. In some embodiments, inhibiting the level or activity PLK1 in the somatic cell increases the rate or efficiency of neuron generation or production by a factor of at least 2.5 fold, at least 2.6 fold, at least 2.7 fold, at least 2.8 fold, at least 2.9 fold, at least 3 fold, at least 3.3 fold, at least 3.6 fold, at least 3.8 fold, at least 4.1 fold, at least 4.4 fold, at least 4.7 fold, at least 4.8 fold, at least 5.0 fold, at least 5.1 fold, at least 5.4 fold, at least 5.6 fold, at least 5.9 fold, at least 6.0 fold, at least 6.2 fold, at least 6.4 fold, at least 6.5 fold, at least 6.7 fold, at least 6.9 fold, at least 7.0 fold, at least 7.2 fold, at least 7.4 fold, at least 7.7 fold, at least 7.9 fold, at least 8.2 fold, at least 8.5 fold, at least 9.0 fold, at least 9.1 fold, at least 9.2 fold, at least 9.3 fold, at least 9.4 fold, at least 9.5 fold or more. In some embodiments, inhibiting the level or activity PLK1 in the somatic cell increases the rate or efficiency of neuron formation via factor-mediated conversion of the non-neuronal cell into a neuron by a factor of at least 10 fold or more compared to forced expression of transdifferentiating transcription factors.

In an aspect, a method for improving the efficiency of neuron generation or production (e.g., motor neuron generation or production) from a somatic cell comprises inhibiting the level or activity of Polio-like kinase I (PLK1) in the somatic cell, thereby increasing the rate or efficiency of neuron generation or production from the somatic cell, in some embodiments, inhibiting the level or activity PLK1 in the somatic cell increases the rate or efficiency of generation or production via factor-mediated conversion of the somatic cell into a neuron by a factor of at least 2.5 fold, at least 2.6 fold, at least 2.7 fold, at least 2.8 fold, at least 2.9 fold, at least 3 fold, at least 3.3 fold, at least 3.6 fold, at least 3.8 fold, at least 4.1 fold, at least 4.4 fold, at least 4.7 fold, at least 4.8 fold, at least 5.0 fold, at least 5.1 fold, at least 5.4 fold, at least 5.6 fold, at least 5.9 fold, at least 6.0 fold, at least 6.2 fold, at least 6.4 fold, at least 6.5 fold, at least 6.7 fold, at least 6.9 fold, at least 7.0 fold, at least 7.2 fold, at least 7.4 fold, at least 7.7 fold, at least 7.9 fold, at least 8.2 fold, at least 8.5 fold, at least 9.0 fold, at least 9.1 fold, at least 9.2 fold, at least 9.3 fold, at least 9.4 fold, at least 9.5 fold or more. In some embodiments, inhibiting the level or activity PLK1 in the somatic cell increases the rate or efficiency of neuron generation or production from a somatic cell via factor-mediated conversion of the somatic cell into a neuron by a factor of at least 10 fold or more compared to forced expression of transdifferentiating transcription factors.

In an aspect, the disclosure provides a method for converting a non-neuronal cell (e.g., somatic cell) into a neuron (e.g., motor neuron) by inhibiting the level or activity of ALK4, ALK5, ALK7, and PLK1 in the somatic cell.

In some embodiments, a method for converting (e.g., transdifferentiating) a non-neuronal cell (e.g., somatic cell) into a neuron comprises inhibiting the level or activity of ALK4, ALK5, ALK7 and PLK1 in the non-neuronal cell, thereby converting the non-neuronal cell into a neuron, wherein the neuron exhibits at least two characteristics of a functional neuron.

In an aspect, a method for improving the efficiency of inducing the generation of neurons (e.g., motor neurons) from non-neuronal cell types comprises inhibiting the level or activity of ALK4, ALK5, ALK7 and PLK1 in the non-neuronal cell, thereby increasing the rate or efficiency of neuron formation. In some embodiments, inhibiting the level or activity ALK4, ALK5, ALK7 and PLK1 in the non-neuronal cell increases the rate or efficiency of neuron formation via factor-mediated conversion of the somatic cell into a neuron by a factor of at least 2.5 fold, at least 2.6 fold, at least 2.7 fold, at least 2.8 fold, at least 2.9 fold, at least 3 fold, at least 3.3 fold, at least 3.6 fold, at least 3.8 fold, at least 4.1 fold, at least 4.4 fold, at least 4.7 fold, at least 4.8 fold, at least 5.0 fold, at least 5.1 fold, at least 5.4 fold, at least 5.6 fold, at least 5.9 fold, at least 6.0 fold, at least 6.2 fold, at least 6.4 fold, at least 6.5 fold, at least 6.7 fold, at least 6.9 fold, at least 7.0 fold, at least 7.2 fold, at least 7.4 fold, at least 7.7 fold, at least 7.9 fold, at least 8.2 fold, at least 8.5 fold, at least 9.0 fold, at least 9.1 fold, at least 9.2 fold, at least 9.3 fold, at least 9.4 fold, at least 9.5 fold or more. In some embodiments, inhibiting the level or activity ALK4, ALK5, ALK7 and PLK1 in the non-neuronal cell increases the rate of neuron formation via factor-mediated conversion of the non-neuronal cell into a neuron by a factor of at least 10 fold or more compared to forced expression of transdifferentiating transcription factors.

In an aspect, the disclosure provides a method for converting (e.g., transdifferentiating) a somatic cell into a motor neuron by inhibiting the level or activity of ALK4, ALK5, ALK7, and PLK1 in the somatic cell. In some embodiments, a method for converting a somatic cell into a motor neuron comprises inhibiting the level or activity of ALK4, ALK5, ALK7 and PLK1 in the somatic cell, thereby converting the somatic cell into a motor neuron, wherein the motor neuron exhibits at least two characteristics of a functional motor neuron.

In an aspect, a method for improving the efficiency of motor neuron generation or production from a somatic cell comprises inhibiting the level or activity of ALK4, ALK5, ALK7 and PLK1 in the somatic cell, thereby increasing the rate or efficiency of motor neuron formation. In some embodiments, inhibiting the level or activity ALK4, ALK5, ALK7 and PLK1 in the somatic cell increases the rate or efficiency of motor neuron formation via factor-mediated conversion of the somatic cell into a motor neuron by a factor of at least 2.5 fold, at least 2.6 fold, at least 2.7 fold, at least 2.8 fold, at least 2.9 fold, at least 3 fold, at least 3.3 fold, at least 3.6 fold, at least 3.8 fold, at least 4.1 fold, at least 4.4 fold, at least 4.7 fold, at least 4.8 fold, at least 5.0 fold, at least 5.1 fold, at least 5.4 fold, at least 5.6 fold, at least 5.9 fold, at least 6.0 fold, at least 6.2 fold, at least 6.4 fold, at least 6.5 fold, at least 6.7 fold, at least 6.9 fold, at least 7.0 fold, at least 7.2 fold, at least 7.4 fold, at least 7.7 fold, at least 7.9 fold, at least 8.2 fold, at least 8.5 fold, at least 9.0 fold, at least 9.1 fold, at least 9.2 fold, at least 9.3 fold, at least 9.4 fold, at least 9.5 fold or more. In some embodiments, inhibiting the level or activity ALK4, ALK5, ALK7 and PLK1 in the somatic cell increases the rate or efficiency of motor neuron formation via factor-mediated conversion of the somatic cell into a motor neuron by a factor of at least 10 fold or more compared to forced expression of transdifferentiating transcription factors.

In this and other aspects described herein, the non-neuronal cell converts (e.g., transdifferentiates) directly from a non-neuronal cell to a neuron. In this and other aspects described herein, the non-neuronal cell converts into a neuron in the absence of exogenous transcription factors. In this and other aspects described herein, the non-neuronal cell converts into a neuron (iN) without the non-neuronal cell becoming an iPS intermediate prior to being converted into the neuron.

In this and other aspects described herein, the somatic cell transdifferentiates directly from a somatic cell to a motor neuron. In this and other aspects described herein, the somatic cell transdifferentiates into a motor neuron in the absence of exogenous transcription factors. In this and other aspects described herein, the somatic cell transdifferentiates into a motor neuron (iMN) without the somatic cell becoming an iPS intermediate prior to being transdifferentiated into the motor neuron.

In some embodiments of this and other aspects described herein, the method comprises increasing the expression of least one MN-inducing factors selected from any of: Lhx3, Ascl1, Brn2, Myt1l, Isl1, Hb9, Ngn2 or NeuroD1, as is described in detail in PCT International Application WO2013/025963, which is incorporated herein by reference in its entirety. In some embodiments of this and other aspects described herein, the method comprises increasing the expression of least two MN-inducing factors selected from any of: Lhx3, Ascl1, Brn2, Myt1l, Isl1, Hb9, Ngn2 or NeuroD1. In some embodiments of this and other aspects described herein, the method comprises increasing the expression of least three MN-inducing factors selected from any of: Lhx3, Ascl1, Brn2, Myt1l, Isl1, Hb9, Ngn2 or NeuroD1.

In some embodiments of this and other aspects described herein, the method comprises increasing the expression of least four MN-inducing factors selected from any of: Lhx3, Ascl1, Brn2, Myt1l, Isl1, Hb9, Ngn2 or NeuroD1. In some embodiments of this and other aspects described herein, the method comprises increasing the expression of least five MN-inducing factors selected from any of: Lhx3, Ascl1, Brn2, Myt1l, Isl1, Hb9, Ngn2 or NeuroD1. In some embodiments of this and other aspects described herein, the method comprises increasing the expression of least six MN-inducing factors selected from any of: Lhx3, Ascl1, Brn2, Myt1l, Isl1, Hb9, Ngn2 or NeuroD1. In some embodiments of this and other aspects described herein, the method comprises increasing the expression of least seven MN-inducing factors selected from any of: Lhx3, Ascl1, Brn2, Myt1l, Isl1, Hb9, Ngn2 or NeuroD1.

In some embodiments of this and other aspects described herein, transcription factor mediated conversion of the non-neuronal cell (e.g., somatic cell) to an iN comprises increasing the expression of least one MN-inducing factors selected from any of; Lhx3, Ascl1, Brn2, Myt1l, Isl1, Hb9, Ngn2 or NeuroD1, as is described in detail in PCT International Application WO2013/025963, which is incorporated herein by reference in its entirety. In some embodiments of this and other aspects described herein, transcription factor mediated conversion of the non-neuronal cell (e.g., somatic cell) to an iN comprises increasing the expression of least two MN-inducing factors selected from any of: Lhx3, Ascl1, Brn2, Myt1l, Isl1, Hb9, Ngn2 or NeuroD1. In some embodiments of this and other aspects described herein, transcription factor mediated conversion of the non-neuronal cell (e.g., somatic cell) to an iN comprises increasing the expression of least one MN-inducing factors selected from any of: Lhx3, Ascl1, Brn2, Myt1l, Isl1, Hb9, Ngn2 or NeuroD1. In some embodiments of this and other aspects described herein, transcription factor mediated conversion of the non-neuronal cell (e.g., somatic cell) to an iN comprises increasing the expression of least three MN-inducing factors selected from any of: Lhx3, Ascl1, Brn2, Myt1l, Isl1, Hb9, Ngn2 or NeuroD1. In some embodiments of this and other aspects described herein, transcription factor mediated conversion of the non-neuronal cell (e.g., somatic cell) to an iN comprises increasing the expression of least four MN-inducing factors selected from any of: Lhx3, Ascl1, Brn2, Myt1l, Isl1, Hb9, Ngn2 or NeuroD1. In some embodiments of this and other aspects described herein, transcription factor mediated conversion of the non-neuronal cell (e.g., somatic cell) to an iN comprises increasing the expression of least five MN-inducing factors selected from any of: Lhx3, Ascl1, Brn2, Myt1l, Isl1, Hb9, Ngn2 or NeuroD1. In some embodiments of this and other aspects described herein, transcription factor mediated conversion of the non-neuronal cell (e.g., somatic cell) to an iN comprises increasing the expression of least six MN-inducing factors selected from any of: Lhx3, Ascl1, Brn2, Myt1l, Isl1, Hb9, Ngn2 or NeuroD1. In some embodiments of this and other aspects described herein, transcription factor mediated conversion of the non-neuronal cell (e.g., somatic cell) to an iN comprises increasing the expression of least seven MN-inducing factors selected from any of: Lhx3, Ascl1, Brn2, Myt1l, Isl1, Hb9, Ngn2 or NeuroD1.

In some embodiments of this and other aspects described herein, transcription factor mediated conversion of the non-neuronal cell (e.g., somatic cell) to an iMN comprises increasing the expression of least one MN-inducing factors selected from any of: Lhx3, Ascl1, Brn2, Myt1l, Isl1, Hb9, Ngn2 or NeuroD1, as is described in detail in PCT International Application WO2013/025963, which is incorporated herein by reference in its entirety. In some embodiments of this and other aspects described herein, transcription factor mediated conversion of the non-neuronal cell (e.g., somatic cell) to an iMN comprises increasing the expression of least two MN-inducing factors selected from any of: Lhx3, Ascl1, Brn2, Myt1l, Isl1, Hb9, Ngn2 or NeuroD1. In some embodiments of this and other aspects described herein, transcription factor mediated conversion of the non-neuronal cell (e.g., somatic cell) to an iMN comprises increasing the expression of least one MN-inducing factors selected from any of: Lhx3, Ascl1, Brn2, Myt1l, Isl1, Hb9, Ngn2 or NeuroD1. In some embodiments of this and other aspects described herein, transcription factor mediated conversion of the non-neuronal cell (e.g., somatic cell) to an iMN comprises increasing the expression of least three MN-inducing factors selected from any of: Lhx3, Ascl1, Brn2, Myt1l, Isl1, Hb9, Ngn2 or NeuroD1. In some embodiments of this and other aspects described herein, transcription factor mediated conversion of the non-neuronal cell (e.g., somatic cell) to an iMN comprises increasing the expression of least four MN-inducing factors selected from any of: Lhx3, Ascl1, Brn2, Myt1l, Isl1, Hb9, Ngn2 or NeuroD1. In some embodiments of this and other aspects described herein, transcription factor mediated conversion of the non-neuronal cell (e.g., somatic cell) to an iMN comprises increasing the expression of least five MN-inducing factors selected from any of: Lhx3, Ascl1, Brn2, Myt1l, Isl1, Hb9, Ngn2 or NeuroD1. In some embodiments of this and other aspects described herein, transcription factor mediated conversion of the somatic cell to an iMN comprises increasing the expression of least six MN-inducing factors selected from any of: Lhx3, Ascl1, Brn2, Myt1l, Isl1, Hb9, Ngn2 or NeuroD1. In some embodiments of this and other aspects described herein, transcription factor mediated conversion of the non-neuronal cell (e.g., somatic cell) to an iMN comprises increasing the expression of least seven MN-inducing factors selected from any of: Lhx3, Ascl1, Brn2, Myt1l, Isl1, Hb9, Ngn2 or NeuroD1.

In some embodiments, an isolated population of iNs produced by the methods and compositions as disclosed herein is a mammalian iN, for example, a human iN.

In some embodiments, an isolated population of iMNs produced by the methods and compositions as disclosed herein is a mammalian iMN, for example, a human iMN.

In some embodiments, an isolated population of induced neurons (iNs) and compositions are produced by a method comprising contacting a cell or a population of a non-neuronal cell (e.g., somatic cell, e.g., fibroblast) with an agent, such as a nucleic acid agent, peptide, polypeptide aptamer, antibody, antibody fragment, ribosomes, small molecules, RNAi agents, ribosomes and the like, which inhibits the level of activity of ALK4, ALK5, and ALK7 in the non-neuronal cell (e.g., somatic cell).

In some embodiments, an isolated population of iMNs and compositions are produced by a method comprising contacting a cell or a population of a non-neuronal cell (e.g., somatic cell, e.g., fibroblast) with an agent, such as a nucleic acid agent, peptide, polypeptide aptamer, antibody, antibody fragment, ribosomes, small molecules, RNAi agents, ribosomes and the like, which inhibits the level of activity of ALK4, ALK5, and ALK7 in the non-neuronal cell (e.g., somatic cell).

In some embodiments, the agent which inhibits the level or activity of ALK4, ALK5, and ALK7 is not A83-01. In some embodiments, the compositions and methods described herein exclude A83-01. In some embodiments, the agent which inhibits the level or activity of ALK4, ALK5, and ALK7 is not SB431542. In some embodiments, the compositions and methods described herein exclude SB431542.

In some embodiments, the agent which inhibits the level or activity of ALK4, ALK5, and ALK7 comprises RepSox.

In some embodiments, the agent which inhibits the level or activity of ALK4, ALK5, and ALK7 comprises an analog or derivative of RepSox.

Exemplary analogs or derivatives of RepSox include, but are not limited compounds other than RepSox of formula (I):

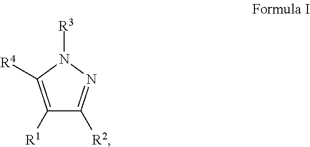

wherein R¹ cyclyl, heterocyclcyl, aryl or heteroaryl, each of which can be optionally substituted; R² cyclyl, heterocyclcyl, aryl or heteroaryl, each of which can be optionally substituted; R³ is H, C₁-C₆ alkyl, arylC₁-C₆, or a nitrogen protecting group, each of which can be optionally substituted; and R⁴ is H, optionally substituted C₁-C₆ alkyl, optionally substituted C₂-C₆ alkenyl, optionally substituted C₂-C₆ alkynyl, or R³ and R⁴ together with the atoms they are attached to form a cyclyl, heterocyclyl, aryl or heteroaryl, each of which can be optionally substituted, as is described further in U.S. Patent Publication No. 2012/0021519, incorporated by reference herein in its entirety.

In some embodiments, the analog or derivative of RepSox comprises a compound other than RepSox selected from the group consisting of: 4-[2-(6-Ethyl-pyridin-2-yl)-pyrazolo[1,5-a]pyridin-3-yl]-quinoline; [2-(6-Methyl-pyridin-2-yl)-pyrazolo[1,5-a]pyridin-3-yl]-quinoline-7-carboxylic acid methyl ester; 4-[2-(6-Methyl-pyridin-2-yl)-pyrazolo[1,5-a]pyridin-3-yl]-quinoline-6-carboxylic acid methyl ester; 4-(5-Benzyl-2-pyridin-2-yl-pyrazolo[1,5-a]pyridin-3-yl)-quinoline-7-carboxylic acid methyl ester; 3-(4-Fluoro-phenyl)-2-(6-methyl-pyridin-2-yl)-pyrazolo[1,5-a]pyridine-6-carboxylic acid (2-dimethylamino-ethyl)-amide; 4-[2-(6-Methyl-pyridin-2-yl)-pyrazolo[1,5-a]pyridin-3-yl]quinoline-6-carboxylic acid (2-dimethylamino-ethyl)-amide; 4-[2-(6-Methyl-pyridin-2-yl)-pyrazolo[1,5-a]pyridin-3-yl]-quinoline-7-carboxylic acid (2-dimethylamino-ethyl)-amide; 5-[2-(6-Methyl-pyridin-2-yl)-pyrazolo[1,5-a]pyridin-3-yl]-benzofuran-2-carboxylic acid (2-dimethyl amino-ethyl)-amide; 4-[2-(6-Methyl-pyridin-2-yl)-pyrazolo[1,5-a]pyridin-3-yl]-quinoline-7-carboxylic acid [3-(4-methyl-piperazin-1-yl)-propyl]-amide; 4-[2-(6-Methoxy-pyridin-2-yl)-pyrazolo[1,5-a]pyridin-3-yl]-quinoline, 4-[2-(6-Ethoxy-pyridin-2-yl)-pyrazolo[1,5-a]pyridin-3-yl]-quinoline; 3-(4-Fluoro-phenyl)-2-(6-methoxy-pyridin-2-yl)-pyrazolo[1,5-a]pyridine; 2-(6-Ethoxy-pyridin-2-yl)-3-(4-fluoro-phenyl)-pyrazolo[1,5-a]pyridine; 7-Benzyl-4-[2-(6-methyl-pyridin-2-yl)-pyrazolo[1,5-a]pyridin-3-yl]-quinoline; 3-{4-[2-(6-Methyl-pyridin-2-yl)-pyrazolo[1,5-a]pyridin-3-yl]-quinolin-7-yl}-acrylic acid methyl ester; 3-{4-[2-(6-Methyl-pyridin-2-yl)-pyrazolo[1,5-a]pyridin-3-yl]-quinolin-7-yl}-acrylic acid; 4-[2-(6-Ethylsulfanyl-pyridin-2-yl)-pyrazolo[1,5-a]-pyridin-3-yl]-quinoline; 4-[2-(6-Phenylsulfanyl-pyridin-2-yl)-pyrazolo[1,5-a]pyridin-3-yl]-quinoline; 4-[2-(6-Morpholin-4-yl-pyridin-2-yl)-pyrazolo[1,5-a]pyridin-3-yl]-quinoline; 3-(4-Fluoro-phenyl)-2-(6-methylsulfanyl-pyridin-2-yl)-pyrazolo[1,5-a]pyridine; 3-(4-Methylsulfanyl-phenyl)-2-(6-methylsulfanyl-pyridin-2-yl)-pyrazolo[1,5-a]pyridine; Dimethyl-{4-[2-(6-methyl-pyridin-2-yl)-pyrazolo[1,5-a]pyridin-3-yl]-quinolin-7-ylsulfanyl}-ethyl)-amine; 2-(Pyridin-2-yl)-3-(quinolin-4-yl)-pyrazolo[1,5-a]pyridine-5-carboxylic acid dimethylamide; 2-(Pyridin-2-yl)-3-(quinolin-4-yl)-pyrazolo[1,5-a]pyridine-6-carboxylic acid dimethylamide; 4-[2-(6-Vinyl-pyridin-2-yl)-pyrazolo[1,5-a]pyridin-3-yl]-quinoline, 6-[2-(6-Methyl-pyridin-2-yl)-pyrazolo[1,5-a]pyridin-3-yl]-imidazo[1,2-a]pyridin-2-yl-amine; 6-[2-(6-Methyl-pyridin-2-yl)-pyrazolo[1,5-a]pyridin-3-yl]-1H-benzoimidazol-2-yl-amine; [3-(4-Fluoro-phenyl)-2-(6-methyl-pyridin-2-yl)-pyrazolo[1,5-a]pyridin-6-yl]-methanol, 6-Allyloxymethyl-3-(4-fluoro-phenyl)-2-(6-methyl-pyridin-2-yl)-pyrazolo[1,5-a]pyridine; 4-[2-(6-Methyl-pyridin-2-yl)-pyrazolo[1,5-a]pyridin-3-yl]-quinoline-7-carboxylic acid (3-pyrrolidin-1-yl-propyl)-amide; 3-{4-[2-(6-Methyl-pyridin-2-yl)-pyrazolo[1,5-a]pyridin-3-yl]-quinolin-7-yl}-propionamide; 3-{4-[2-(6-Methyl-pyridin-2-yl)-pyrazolo[1,5-a]pyridin-3-yl]-quinolin-7-yl}-N-(3-pyrrolidin-1-yl-propyl)-propionamide; N-(Dimethylamino-ethyl)-3-{4-[2-(6-methyl-pyridin-2-yl)-pyrazolo[1,5-a]pyridin-3-yl]-quinolin-7-yl}-propionamide; 2-Pyridin-2-yl-3-quinolin-4-yl-pyrazolo[1,5-a]pyridine-5-carboxylic acid (3-dimethylamino-propyl)-amide; 4-[2-(6-Methyl-pyridin-2-yl)-pyrazolo[1,5-a]pyridin-3-yl]-quinoline-7-carboxylic acid (2-hydroxy-ethyl)-amide; 4-[2-(6-Methyl-pyridin-2-yl)-pyrazolo[1,5-a]pyridin-3-yl]-quinoline-7-carboxylic acid hydrazide; 4-[2-(6-Methyl-pyridin-2-yl)-pyrazolo[1,5-a]pyridin-3-yl]-quinoline-7-carboxylic acid (3-hydroxy-propyl)-amide; 4-[2-(6-Methyl-pyridin-2-yl)-pyrazolo[1,5-a]pyridin-3-yl]-quinoline-7-carboxylic acid methylamide; 4-[2-(6-Methyl-pyridin-2-yl)-pyrazolo[1,5-a]pyridin-3-yl]-quinoline-7-carboxylic acid (3-ethoxy-propyl)-amide; 4-[2-(6-Methyl-pyridin-2-yl)-pyrazolo[1,5-a]pyridin-3-yl]-quinoline-7-carboxylic acid (3-morpholin-4-yl-propyl)-amide; 4-[2-(6-Methyl-pyridin-2-yl)-pyrazolo[1,5-a]pyridin-3-yl]quinoline-7-carboxylic acid (3-imidazol-1-yl-propyl)-amide; 4-[2-(6-Methyl-pyridin-2-yl)-pyrazolo[1,5-a]pyridin-3-yl]-quinoline-7-carboxylic acid (3-dimethylamino-propyl)-amide; 4-[2-(6-Methyl-pyridin-2-yl)-pyrazolo[1,5-a]pyridin-3-yl]-quinoline-7-carboxylic acid [2-(2-methoxy-phenyl)-ethyl]-amide; 4-[2-(6-Methyl-pyridin-2-yl)-pyrazolo[1,5-a]pyridin-3-yl]-quinoline-7-carboxylic acid (2-morpholin-4-yl-ethyl)-amide; 4-[2-(6-Methyl-pyridin-2-yl)-pyrazolo[1,5-a]pyridin-3-yl]quinoline-7-carboxylic acid amide; Dimethyl-{4-[2-(6-methyl-pyridin-2-yl)-pyrazolo[1,5-a]pyridin-3-yl]-quinolin-7-yloxy}-propyl)-amine; 4-[2-(6-Methyl-pyridin-2-yl)-pyrazolo[1,5-a]pyridin-3-yl]-7-(2-morpholin-4-yl-ethoxy)-quinoline; Diisopropyl-(2-{4-[2-(6-methyl-pyridin-2-yl)-pyrazolo[1,5-a]pyridin-3-yl]-quinolin-7-yloxy}-ethyl)-amine; 4-[2-(6-Methyl-pyridin-2-yl)-pyrazolo[1,5-a]pyridin-3-yl]-7-(2-pyrrol-1-yl-ethoxy)-quinoline; Dimethyl-(1-methyl-2-{4-[2-(6-methyl-pyridin-2-yl)-pyrazolo[1,5-a]pyridin-3-yl]-quinolin-7-yloxy}ethyl)-amine; Methyl-(3-{4-[2-(6-methyl-pyridin-2-yl)-pyrazolo[1,5-a]pyridin-3-yl]-quinolin-7-yl-oxy}-propyl)-amine; 4-[2-(6-Methyl-pyridin-2-yl)-pyrazolo[1,5-a]pyridin-3-yl]-7-(2-piperidin-1-yl-ethoxy)-quinoline; Diethyl-(2-{4-[2-(6-methyl-pyridin-2-yl-pyrazolo[1,5-a]pyridin-3-yl]-quinolin-7-yloxy}-ethyl)-amine; Dimethyl-{3-[4-(2-pyridin-2-yl-pyrazolo[1,5-a]pyridin-3-yl)-quinolin-7-yloxy]-propyl}-amine; 7-(2-Morpholin-4-yl-ethoxy)-4-(2-pyridin-2-yl-pyrazolo[1,5-a]pyridin-3-yl)-quinoline; Diisopropyl-{2-[4-(2-pyridin-2-yl-pyrazolo[1,5-a]pyridin-3-yl)-quinolin-7-yloxy]-ethyl}-amine; 4-[2-(6-Methyl-pyridin-2-yl)-pyrazolo[1,5-a]pyridin-3-yl]-7-(3-morpholin-4-yl-propoxy)-quinoline; (3-{4-[2-(6-Methyl-pyridin-2-yl)-pyrazolo[1,5-a]pyridine-3-yl]-quinolin-7-yloxy}-propyl)-1,3-dihydro-benzoimidazol-2-one 3-{4-[2-(6-Methyl-pyridin-2-yl)-pyrazolo[1,5-a]pyridin-3-yl]-quinolin-7-yl}-propionic acid methyl ester; Diethyl-3-[4-(2-pyridin-2-yl-pyrazolo[1,5-a]pyridin-3-yl)-quinolin-7-yloxy]-propyl}-amine; Ethyl-methyl-{3-[4-(2-pyridin-2-yl-pyrazolo[1,5-a]pyridin-3-yl)-quinolin-7-yloxy]-propyl}-amine; 4-(2-Pyridin-2-yl-pyrazolo[1,5-a]pyridin-3-yl)-7-(3-pyrrolidin-1-yl-propoxy)-quinoline; 7-(3-Piperidin-1-yl-propoxy)-4-(2-pyridin-2-yl-pyrazolo[1,5-a]pyridin-3-yl)-quinoline; Diethyl-{2-[4-(2-pyridin-2-yl-pyrazolo[1,5-a]pyridin-3-yl)-quinolin-7-yloxy]-ethyl}-amine; Dimethyl-{2-[4-(2-pyridin-2-yl-pyrazolo[1,5-a]pyridin-3-yl)-quinolin-7-yloxy]-ethyl}-amine; 6-Bromo-4-(2-pyridin-2-yl-5,6-dihydro-4H-pyrrolo[1,2-b]pyrazol-3-yl)-quinoline; 3-Pyridin-4-yl-2-pyridin-2-yl-5,6-dihydro-4H-pyrrolo[1,2-b]pyrazole; 2-(6-Methyl-pyridin-2-yl)-3-p-tolyl-5,6-dihydro-4H-pyrrolo[1,2-b]pyrazole; 4-[3-(6-Methyl-pyridin-2-yl)-5,6-dihydro-4H-pyrrolo[1,2-b]pyrazol-2-yl]-quinoline; 2-(6-Methyl-pyridin-2-yl)-3-naphthalen-1-yl-5,6-dihydro-4H-pyrrolo[1,2-b]pyrazole; (6-Methyl-pyridin-2-yl)-3-pyridin-3-yl-5,6-dihydro-4H-pyrrolo[1,2-b]pyrazole; 3-(4-Fluoro-naphthalen-1-yl)-2-(6-methyl-pyridin-2-yl)-5,6-dihydro-4H-pyrrolo[1,2-b]pyrazole; 3-(3,4-Difluoro-phenyl)-2-(6-methyl-pyridin-2-yl)-5,6-dihydro-4H-pyrrolo[1,2-b]pyrazole; [2-(4-Methanesulfonyl-phenyl)-1-(6-methyl-pyridin-2-yl)-ethylideneamino]-pyrrolidin-2-one; 7-Methoxy-4-(2-pyridin-2-yl-5,6-dihydro-4H-pyrrolo[1,2-b]pyrazol-3-yl)-quinoline; 7-Benzyloxy-6-methoxy-4-(2-pyridin-2-yl-5,6-dihydro-4H-pyrrolo[1,2-b]pyrazol-3-yl)-quinoline; 6-(2-Pyridin-2-yl-5,6-dihydro-4H-pyrrolo[1,2-b]pyrazol-3-yl)-quinoline; 6-[2-(6-Methyl-pyridin-2-yl)-5,6-dihydro-4H-pyrrolo[1,2-b]pyrazol-3-yl]-quinoline; 3-Naphthalen-2-yl-2-pyridin-2-yl-5,6-dihydro-4H-pyrrolo[1,2-b]pyrazole; 2-(6-Methyl-pyridin-2-yl)-3-naphthalen-2-yl-5,6-dihydro-4H-pyrrolo[1,2-b]pyrazole; 3-(4-Fluoro-phenyl)-2-(6-trifluoromethyl-pyridin-2-yl)-5,6-dihydro-4H-pyrrolo[1,2-b]pyrazole; 4-(Quinolin-4-yl)-3-(5-fluoropyridin-2-yl)-5,6-dihydro-4H-pyrrolo[1,2-b]pyrazole; 4-(7-Bromoquinolin-4-yl)-3-(pyridin-2-yl)-5,6-dihydro-4H-pyrrolo[1,2-b]pyrazole; (Quinolin-4-yl)-3-(2,4-difluorophenyl)-5,6-dihydro-4H-pyrrolo[1,2-b]pyrazole; 4-(2-Pyrazin-2-yl-5,6-dihydro-4H-pyrrolo[1,2-b]pyrazol-3-yl)-quinoline; 4-(5-Methyl-2-pyridin-2-yl-5,6-dihydro-4H-pyrrolo[1,2-b]pyrazol-3-yl)-quinoline; 6-Bromo-4-[2-(6-methyl-pyridin-2-yl)-5,6-dihydro-4H-pyrrolo[1,2-b]pyrazol-3-yl]-quinoline; 4-[2-(6-Methyl-pyridin-2-yl)-5,6-dihydro-4H-pyrrolo[1,2-b]pyrazol-3-yl]-6-trifluoromethyl-quinoline; 3-(3-Chloro-4-fluoro-phenyl)-2-(6-methyl-pyridin-2-yl)-5,6-dihydro-4H-pyrrolo[1,2-b]pyrazole; 3-(2-Chloro-4-fluoro-phenyl)-2-(6-methyl-pyridin-2-yl)-5,6-dihydro-4H-pyrrolo[1,2-b]pyrazole; 3-(4-Fluoro-3-trifluoromethyl-phenyl)-2-(6-methyl-pyridin-2-yl)-5,6-dihydro-4H-pyrrolo[1,2-b]pyrazole; 2-(6-Methyl-pyridin-2-yl)-3-(2,4,5-trifluoro-phenyl)-5,6-dihydro-4H-pyrrolo[1,2-b]pyrazole; 8-Fluoro-4-[2-(6-methyl-pyridin-2-yl)-5,6-dihydro-4H-pyrrolo[1,2-b]pyrazol-3-yl]-quinoline; 7-Bromo-4-[2-(6-methyl-pyridin-2-yl)-5,6-dihydro-4H-pyrrolo[1,2-b]pyrazol-3-yl]-quinoline; 4-[2-(6-Methyl-pyridin-2-yl)-5,6-dihydro-4H-pyrrolo[1,2-b]pyrazol-3-yl]-6-trifluoromethoxy-quinoline; 4-[2-(6-Methyl-pyridin-2-yl)-5,6-dihydro-4H-pyrrolo[1,2-b]pyrazol-3-yl]-7-trifluoromethyl-quinoline; 7-Methoxy-4-[2-(6-methyl-pyridin-2-yl)-5,6-dihydro-4H-pyrrolo[1,2-b]pyrazol-3-yl]-quinoline; 3-(2-Chloro-pyridin-4-yl)-2-pyridin-2-yl-5,6-dihydro-4H-pyrrolo[1,2-h]pyrazole; [2-(6-Methyl-pyridin-2-yl)-3-quinolin-4-yl-5,6-dihydro-4H-pyrrolo[1,2-b]pyrazol-6-yl]-methanol; [3-(7-Bromo-quinolin-4-yl)-2-(6-methyl-pyridin-2-yl)-5,6-dihydro-4H-pyrrolo[1,2-b]pyrazol-6-yl]-methanol; 4-[2-(6-Chloro-pyridin-2-yl)-5-(4-fluorophenyl)-5,6-dihydro-4H-pyrrolo[1,2-b]pyrazol-3-yl]-quinoline; 4-[2-(6-Ethoxy-pyridin-2-yl)-5-(4-fluoro-phenyl)-5,6-dihydro-4H-pyrrolo[1,2-b]pyrazol-3-yl]-quinoline; (S)-4-[6-Benzyloxymethyl-2-(6-methyl-pyridin-2-yl)-5,6-dihydro-4H-pyrrolo[1,2-b]pyrazol-3-yl]-7-chloro-quinoline; (S)-4-[6-Benzyloxymethyl-2-(6-chloro-pyridin-2-yl)-5,6-dihydro-4H-pyrrolo[1,2-b]pyrazol-3-yl]-quinoline; 4-[2-(6-Methyl-pyridin-2-yl)-3-quinolin-4-yl-5,6-dihydro-4H-pyrrolo[1,2-b]pyrazol-5-yl]-benzoic acid ethyl ester; 3-(4-Fluoro-phenyl)-5,5-dimethyl-2-(6-methyl-pyridin-2-yl)-5,6-dihydro-4H-pyrrolo[1,2-b]pyrazole; (R)-6-Benzyloxymethyl-3-(4-fluoro-phenyl)-2-(6-methyl-pyridin-2-yl)-5,6-dihydro-4H-pyrrolo[1,2-b]pyrazole; 5-(4-Chloro-phenyl)-3-(4-fluoro-phenyl)-2-(6-methyl-pyridin-2-yl)-5,6-dihydro-4H-pyrrolo[1,2-b]pyrazole; 4-[2-(3-Trifluoromethyl-phenyl)-4,5,6,7-tetrahydro-pyrazolo[1,5-a]pyridin-3-yl]-quinoline; 4-[2-(4-Trifluoromethyl-phenyl)-4,5,6,7-tetrahydro-pyrazolo[1,5-a]pyridin-3-yl]-quinoline; 4-[2-(4-Chloro-phenyl)-4,5,6,7-tetrahydro-pyrazolo[1,5-a]pyridin-3-yl]-quinoline; 4-[2-(3-Chloro-phenyl)-4,5,6,7-tetrahydro-pyrazolo[1,5-a]pyridin-3-yl]-quinoline; 4-[2-(3-Fluoro-5-trifluoromethyl-phenyl)-5,6-dihydro-4H-pyrrolo[1,2-b]pyrazol-3-yl]-quinoline; 4-[2-(3-Fluoro-5-trifluoromethyl-phenyl)-4,5,6,7-tetrahydro-pyrazolo[1,5-a]pyridin-3-yl]-quinoline; 4-(2-Phenyl-4,5,6,7-tetrahydro-pyrazolo[1,5-a]pyridin-3-yl)-quinoline; 4-(2-Pyridin-2-yl-4,5,6,7-tetrahydro-pyrazolo[1,5-a]pyridin-3-yl)-[1,10]phenanthroline; 4-[2-(4-Fluoro-phenyl)-4,5,6,7-tetrahydro-pyrazolo[1,5-a]pyridin-3-yl]-quinoline; 4-[2-(3-Trifluoromethoxy-phenyl)-4,5,6,7-tetrahydro-pyrazolo[1,5-a]pyridin-3-yl]-quinoline; 4-[2-(2-Fluoro-phenyl)-4,5,6,7-tetrahydro-pyrazolo[1,5-a]pyridin-3-yl]-quinoline; 4-(2-Quinolin-2-yl-4,5,6,7-tetrahydro-pyrazolo[1,5-a]pyridin-3-yl)-quinoline; 4-[2-(4-Ethyl-pyridin-2-yl)-4,5,6,7-tetrahydro-pyrazolo[1,5-a]pyridin-3-yl]-quinoline; 4-(2-Quinolin-2-yl-5,6-dihydro-4H-pyrrolo[1,2-b]pyrazol-3-yl)-quinoline; 2-(3-Quinolin-4-yl-4,5,6,7-tetrahydro-pyrazolo[1,5-a]pyridin-2-yl)-[1,8]naphthyridine; 4-[5-(4-Fluoro-phenyl)-2-pyridin-2-yl-5,6-dihydro-4H-pyrrolo[1,2-b]pyrazol-3-yl]-quinoline; 4-(6-Hydroxymethyl-2-pyridin-2-yl-5,6-dihydro-4H-pyrrolo[1,2-b]pyrazol-3-yl)-quinoline; 4-(3-Pyridin-2-yl-5,6-dihydro-4H-pyrrolo[1,2-b]pyrazol-2-yl)-quinoline; 4-(4-Methyl-2-pyridin-2-yl-5,6-dihydro-4H-pyrrolo[1,2-b]pyrazol-3-yl)-quinoline; 4-(5-Benzyl-2-pyridin-2-yl-5,6-dihydro-4H-pyrrolo[1,2-b]pyrazol-3-yl)-quinoline; 4-(5-Phenethyl-2-pyridin-2-yl-5,6-dihydro-4H-pyrrolo[1,2-b]pyrazol-3-yl)-quinoline; 4-(5-Phenyl-2-pyridin-2-yl-5,6-dihydro-4H-pyrrolo[1,2-b]pyrazol-3-yl)-quinoline; 4-[2-(3-Trifluoromethylphenyl)-5,6-dihydro-4H-pyrrolo[1,2-b]pyrazol-3-yl]-quinoline; 4-[2-(4-Trifluoromethyl-phenyl)-5,6-dihydro-4H-pyrrolo[1,2-b]pyrazol-3-yl]-quinoline; 4-(2-Phenyl-5,6-dihydro-4H-pyrrolo[1,2-b]pyrazol-3-yl)-quinoline; 2-Chloro-4-(2-pyridin-2-yl-5,6-dihydro-4H-pyrrolo[1,2-b]pyrazol-3-yl)-quinoline; 6,8-Dimethoxy-4-[2-(6-methyl-pyridin-2-yl)-5,6-dihydro-4H-pyrrolo[1, 2b]pyrazol-3-yl]-quinoline; 4-[2-(6-Bromo-pyridin-2-yl)-5,6-dihydro-4H-pyrrolo[1,2-b]pyrazol-3-yl]-quinoline; 6,8-Dimethoxy-4-[2-pyridin-2-yl-5,6-dihydro-4H-pyrrolo[1, 2b]pyrazol-3-yl]-quinoline; 3-(4-Fluorophenyl)-2-pyridin-2-yl-5,6-dihydro-4H-pyrrolo[1,2-b]pyrazole; 3-(4-Methoxy-phenyl)-2-pyridin-2-yl-5,6-dihydro-4H-pyrrolo[1,2-b]pyrazole; 3-(4-Fluorophenyl)-2-(6-methylpyridin-2-yl)-5,6-dihydro-4H-pyrrolo[1,2-b]pyrazole; 3-(4-Methoxyphenyl)-2-(6-methylpyridin-2-yl)-5,6-dihydro-4H-pyrrolo[1,2-b]pyrazole; 4-(2-Thiophen-2-yl-5,6-dihydro-4H-pyrrolo[1,2-b]pyrazol-3-yl)quinoline; 4-[2-(6-Propylpyridin-2-yl)-5,6-dihydro-4H-pyrrolo[1,2-b]pyrazol-3-yl]-quinoline; 4-[2-(6-Isopropylpyridin-2-yl)-5,6-dihydro-4H-pyrrolo[1,2-b]pyrazol-3-yl]quinoline; 4-[2-(6-Ethyl-pyridin-2-yl)-5,6-dihydro-4H-pyrrolo[1,2-b]pyrazol-3-yl]quinoline; 4-[2-(6-Methyl-pyridin-2-yl)-5,6-dihydro-4H-pyrrolo[1,2-b]pyrazol-3-yl]-quinoline; 4-[2-(3-Fluorophenyl)-5,6-dihydro-4H-pyrrolo[1,2-b]pyrazol-3-yl]-quinoline; 4-[2-(2-Fluoro-phenyl)-5,6-dihydro-4H-pyrrolo[1,2-b]pyrazol-3-yl]-quinoline; 4-[2-(4-Fluoro-phenyl)-5,6-dihydro-4H-pyrrolo[1,2-b]pyrazol-3-yl]-quinoline; 4-[2-(3-Trifluoromethoxy-phenyl)-5,6-dihydro-4H-pyrrolo[1,2-b]pyrazol-3-yl]-quinoline; 4-[2-(4-Chloro-pyridin-2-yl)-5,6-dihydro-4H-pyrrolo[1,2-b]pyrazol-3-yl]-quinoline; 4-[2-(4-Fluoro-3-trifluoromethyl-phenyl)-5,6-dihydro-4H-pyrrolo[1,2-b]pyrazol-3-yl]quinoline; 4-[2-(2-Fluoro-3-trifluoromethyl-phenyl)-5,6-dihydro-4H-pyrrolo[1,2-b]-pyrazol-3-yl]-quinoline; 4-[5-(3-Methoxy-phenyl)-2-pyridin-2-yl-5,6-dihydro-4H-pyrrolo[1,2-b]pyrazol-3-yl]-quinoline; 4-[2-(4-Fluoro-3-trifluoromethyl-phenyl)-5-(3-methoxy-phenyl)-5,6-dihydro-4H-pyrrolo[1,2-b]pyrazol-3-yl]-quinoline; 4-(7-Chloroquinolin-4-yl)-3-(6-methylpyridin-2-yl)-5,6-dihydro-4H-pyrrolo[1,2-b]pyrazole; 4-(7-Ethoxyquinolin-4-yl)-3-(6-methylpyridin-2-yl)-5,6-dihydro-4H-pyrrolo[1,2-h]pyrazole; 6-(3-Quinolin-4-yl-5,6-dihydro-4H-pyrrolo[1,2-b]pyrazol-2-yl)-pyridine-2-carboxylic acid hydrochloride; 6,7-Difluoro-4-[2-(6-methyl-pyridin-2-yl)-5,6-dihydro-4H-pyrrolo[1,2-b]pyrazol-3-yl]-quinoline; 6,7-Dimethoxy-4-[2-(6-methyl-pyridin-2-yl)-5,6-dihydro-4H-pyrrolo[1,2-b]pyrazol-3-yl]-quinoline; 3-Benzo[1, 3]dioxol-5-yl-2-(6-methyl-pyridin-2-yl)-5,6-dihydro-4H-pyrrolo[1,2-b]pyrazole; 6-(4-Fluoro-phenyl)-4-[2-(6-methyl-pyridin-2-yl)-5,6-dihydro-4H-pyrrolo[1,2-b]pyrazol-3-yl]-quinoline; 6-Benzo[lI, 3]dioxol-5-yl-4-[2-(6-methyl-pyridin-2-yl)-5,6-dihydro-4H-pyrrolo[1,2-b]pyrazol-3-yl]-quinoline; 4-[2-(6-Methyl-pyridin-2-yl)-5,6-dihydro-4H-pyrrolo[1,2-b]pyrazol-3-yl]-6-thiophen-2-yl-quinoline; 4-[2-(6-Methyl-pyridin-2-yl)-5,6-dihydro-4H-pyrrolo[1,2-b]pyrazol-3-yl]-6-phenyl-quinoline; 8-[2-(6-Methyl-pyridin-2-yl)-5,6-dihydro-4H-pyrrolo[1,2-b]pyrazol-3-yl]-quinoline; 3-Benzo[b]thiophen-2-yl-2-(6-methy-pyridin-2-yl)-5,6-dihydro-4H-pyrrolo[1,2-b]pyrazole; 4-(2-Pyridin-2-yl-5,6-dihydro-4H-pyrrolo[1,2-b]pyrazol-3-yl)-quinoline-6-carboxylic acid methyl ester; 4-[2-(6-Methyl-pyridin-2-yl)-5,6-dihydro-4H-pyrrolo[1,2-b]pyrazol-3-yl]-quinoline-6-carboxylic acid methyl ester; 4-[2-(6-Methyl-pyridin-2-yl)-5,6-dihydro-4H-pyrrolo[1,2-b]pyrazol-3-yl]-quinoline-7-carboxylic acid methyl ester; 4-[2-Pyridin-2-yl-5,6-dihydro-4H-pyrrolo[1,2-b]pyrazol-3-yl]-quinoline-7-carboxylic acid methyl ester; 2-Pyridin-2-yl-3-quinolin-4-yl-pyrazolo[5,1-c]morpholine; 2-Pyridin-2-yl-3-quinolin-4-yl-pyrazolo[5,1-c]morpholin-4-one; Dimethyl-{3-[4-(2-pyridin-2-yl-5,6-dihydro-4H-pyrrolo[1,2-b]pyrazol-3-yl)-quinolin-7-yloxy]-propyl}-amine; {3-[6-Methoxy-4-(2-pyridin-2-yl-5,6-dihydro-4H-pyrrolo[1,2-b]pyrazol-3-yl)-quinolin-7-yloxy]-propyl}-dimethyl-amine; Cyclopropylmethyl-propyl-{3-[4-(2-pyridin-2-yl-5,6-dihydro-4H-pyrrolo[1,2-b]pyrazol-3-yl)-quinolin-7-yloxy]-propyl}-amine; Diethyl-{3-[4-(2-pyridin-2-yl-5,6-dihydro-4H-pyrrolo[1,2-b]pyrazol-3-yl)-quinolin-7-yloxy]-propyl}-amine; Ethyl-methyl-{3-[4-(2-pyridin-2-yl-5,6-dihydro-4H-pyrrolo[1,2-b]pyrazol-3-yl)-quinolin-7-yloxy]-propyl}-amine)jjjjj) 3-[4-(2-Pyridin-2-yl-5,6-dihydro-4H-pyrrolo[1,2-b]pyrazol-3-yl)-quinolin-7-yloxy]-propylamine; 7-[3-(4-Methyl-piperazin-1-yl)-propoxy]-4-(2-pyridin-2-yl-5,6-dihydro-4H-pyrrolo[1,2-b]pyrazol-3-yl)-quinoline; Benzyl-methyl-{3-[4-(2-pyridin-2-yl-5,6-dihydro-4H-pyrrolo[1,2-b]pyrazol-3-yl)-quinolin-7-yloxy]-propyl}-amine; 7-(3-Piperidin-1-yl-propoxy)-4-(2-pyridin-2-yl-5,6-dihydro-4H-pyrrolo[1,2-b]pyrazol-3-yl)-quinoline; 4-(2-Pyridin-2-yl-5,6-dihydro-4H-pyrrolo[1,2-b]pyrazol-3-yl)-7-(3-pyrrolidin-1-yl-propoxy)-quinoline; 7-(3-Azepan-1-yl-propoxy)-4-(2-pyridin-2-yl-5,6-dihydro-4H-pyrrolo[1,2-b]pyrazol-3-yl)-quinoline; 7-(3-Imidazol-1-yl-propoxy)-4-(2-pyridin-2-yl-5,6-dihydro-4H-pyrrolo[1,2-b]pyrazol-3-yl)-quinoline; 7-(3-Pyrazol-1-yl-propoxy)-4-(2-pyridin-2-yl-5,6-dihydro-4H-pyrrolo[1,2-b]pyrazol-3-yl)-quinoline; 1′-{3-[4-(2-Pyridin-2-yl-5,6-dihydro-4H-pyrrolo[1,2-b]pyrazol-3-yl)-quinolin-7-yloxy]-propyl}-[1,4′]bipiperidinyl; Cyclopropyl-(1-methyl-piperidin-4-yl)-f 3-[4-(2-pyridin-2-yl-5,6-dihydro-4H-pyrrolo[1,2-b]pyrazol-3-yl)-quinolin-7-yloxy]-propyl}-amine; 4-(2-Pyridin-2-yl-5,6-dihydro-4H-pyrrolo[1,2-b]pyrazol-3-yl)-7-(3-[1,2,3]triazol-1-yl-propoxy)-quinoline; Dimethyl-(3-{4-[2-(6-methyl-pyridin-2-yl)-5,6-dihydro-4H-pyrrolo[1,2-b]pyrazol-3-yl]-quinolin-7-yloxy}-propyl)-amine; Diethyl-(3-{4-[2-(6-methyl-pyridin-2-yl)-5,6-dihydro-4H-pyrrolo[1,2-b]pyrazol-3-yl]-quinolin-7-yloxy}-propyl)-amine; Cyclopropylmethyl-(3-{4-[2-(6-methyl-pyridin-2-yl)-5,6-dihydro-4H-pyrrolo[1,2-b]pyrazol-3-yl]-quinolin-7-yloxy}-propyl)-propyl-amine; Ethyl-methyl-(3-{4-[2-(6-methyl-pyridin-2-yl)-5,6-dihydro-4H-pyrrolo[1,2-b]pyrazol-3-yl]-quinolin-7-yloxy}-propyl)-amine; Dimethyl-{2-[4-(2-pyridin-2-yl-5,6-dihydro-4H-pyrrolo[1,2-b]pyrazol-3-yl)-quinolin-7-yloxy]-ethyl}-amine; Diethyl-{2-[4-(2-pyridin-2-yl-5,6-dihydro-4H-pyrrolo[1,2-b]pyrazol-3-yl)-quinolin-7-yloxy]-ethyl}-amine; 7-(2-Piperidin-1-yl-ethoxy)-4-(2-pyridin-2-yl-5,6-dihydro-4H-pyrrolo[1,2-b]pyrazol-3-yl)-quinoline; Ethyl-methyl-{2-[4-(2-pyridin-2-yl-5,6-dihydro-4H-pyrrolo[1,2-b]pyrazol-3-yl)-quinolin-7-yloxy]ethyl}-amine; 4-(2-Pyridin-2-yl-5,6-dihydro-4H-pyrrolo[1,2-b]pyrazol-3-yl)-7-(2-pyrrolidin-1-yl-ethoxy)-quinoline; 7-[2-(4-Methyl-piperazin-1-yl)-ethoxy]-4-(2-pyridin-2-yl-5,6-dihydro-4H-pyrrolo[1,2-b]pyrazol-3-yl)-quinoline; Dimethyl-{3-[1-oxy-4-(2-pyridin-2-yl-5,6-dihydro-4H-pyrrolo[1,2-b]pyrazol-3-yl)-quinolin-7-yloxy]-propyl}-amine; 7-Methylsulfanyl-4-(2-pyridin-2-yl-5,6-dihydro-4H-pyrrolo[1,2-b]pyrazol-3-yl)-quinoline; 7-Ethylsulfanyl-4-(2-pyridin-2-yl-5,6-dihydro-4H-pyrrolo[1,2-b]pyrazol-3-yl)-quinoline; 6-Methylsulfanyl-4-(2-pyridin-2-yl-5,6-dihydro-4H-pyrrolo[1,2-b]pyrazol-3-yl)-quinoline; 7-Benzylsulfanyl-4-(2-pyridin-2-yl-5,6-dihydro-4H-pyrrolo[1,2-b]pyrazol-3-yl)-quinoline; 3-[4-(2-Pyridin-2-yl-5,6-dihydro-4H-pyrrolo[1,2-b]pyrazol-3-yl)-quinolin-7-yl sulfanyl]-propan-1-ol; Dimethyl-{2-[4-(2-pyridin-2-yl-5,6-dihydro-4H-pyrrolo[1,2-b]pyrazol-3-yl)-quinolin-7-ylsulfanyl]-ethyl}-amine; Dimethyl[6-(3-quinolin-4-yl-5,6-dihydro-4H-pyrrolo[1,2-b]pyrazol-2-yl)-pyridin-2-yl-methyl]amine; 7-(2-Propoxy-ethoxy)-4-(2-pyridin-2-yl-5,6-dihydro-4H-pyrrolo[1,2-b]pyrazol-3-yl)-quinoline; N,N-Dimethyl-N′-[4-(2-pyridin-2-yl-5,6-dihydro-4H-pyrrolo[1,2-b]pyrazol-3-yl)-pyridin-2-yl]-ethane-1,2-diamine; N,N-Dimethyl-N′-[4-(2-pyridin-2-yl-5,6-dihydro-4H-pyrrolo[1,2-b]pyrazol-3-yl)-pyridin-2-yl]-propane-1,3-diamine; 3-{3-[4-(2-Pyridin-2-yl-5,6-dihydro-4H-pyrrolo[1,2-b]pyrazol-3-yl)-quinolin-7-yloxy]-propyl}-oxazolidin-2-one; 1-{3-[4-(2-Pyridin-2-yl-5,6-dihydro-4H-pyrrolo-[1,2-b]pyrazol-3-yl)-quinolin-7-yloxy]-propyl}-imidazolidin-2-one; 3-{3-[4-(2-Pyridin-2-yl-5,6-dihydro-4H-pyrrolo[1,2-b]pyrazol-3-yl)-quinolin-7-yloxy]-propyl}-3H-benzooxazol-2-one; Dimethyl-(2-{4-[2-(6-methyl-pyridin-2-yl)-5,6-dihydro-4H-pyrrolo[1,2-b]pyrazol-3-yl]-pyridin-2-ylsulfanyl}-ethyl-amine; 4-(2-Pyridin-2-yl-5,6-dihydro-4H pyrrolo-[1,2-b]pyrazol-3-yl)-2pyrrolidin-1-yl-quinoline; 2-Phenylsulfanyl-4-(2-pyridin-2-yl-5,6-dihydro-4H-pyrrolo[1,2-b]pyrazol-3-yl)-quinoline; 2-Morpholin-4-yl-4-(2-pyridin-2-yl-5,6-dihydro-4H-pyrrolo[1,2-b]pyrazol-3-yl)-quinoline; 2-Ethylsulfanyl-4-(2-pyridin-2-yl-5,6-dihydro-4H-pyrrolo[1,2-b]pyrazol-3-yl)-quinoline; Phenyl-[4-(2-pyridin-2-yl-5,6-dihydro-4H-pyrrolo-[1,2-b]pyrazol-3-yl)-quinolin-2-yl]-amine; 2-Methoxy-4-(2-pyridin-2-yl-5,6-dihydro-4H-pyrrolo[1,2-b]pyrazol-3-yl)-quinoline; 2-Ethoxy-4-(2-pyridin-2-yl-5,6-dihydro-4H-pyrrolo[1,2-b]pyrazol-3-yl)-quinoline; 4-[2-(6-Phenylsulfanyl-pyridin-2-yl)-5,6-dihydro-4H-pyrrolo[1,2-b]pyrazol-3-yl]-quinoline; Phenyl-[6-(3-quinolin-4-yl-5,6-dihydro-4H-pyrrolo[1,2-b]pyrazol-2-yl)-pyridin-2-yl]-amine; 4-{2-[6-(4-Methoxy-phenyl)-pyridin-2-yl]-5,6-dihydro-4H-pyrrolo[1,2-b]pyrazol-3-yl}-quinoline; 4-[2-(6-Phenyl-pyridin-2-yl)-5,6-dihydro-4H-pyrrolo[1,2-b]pyrazol-3-yl]-quinoline; 4-[2-(6-Morpholin-4-yl-pyridin-2-yl)-5,6-dihydro-4H-pyrrolo[1,2-b]pyrazol-3-yl]-quinoline; 4-[2-(6-Pyrrolidin-1-yl-pyridin-2-yl)-5,6-dihydro-4H-pyrrolo[1,2-b]pyrazol-3-yl]-quinoline; 4-[2-(6-Methoxy-pyridin-2-yl)-5,6-dihydro-4H-pyrrolo[1,2-b]pyrazol-3-yl]-quinoline; 2-{3-[4-(2-Pyridin-2-yl-5,6-dihydro-4H-pyrrolo-[1,2-b]pyrazol-3-yl)-quinolin-7-yloxy]-propyl}-isoindole-1,3-dione; 7-(3-Fluoro-propoxy)-4-(2-pyridin-2-yl-5,6-dihydro-4H-pyrrolo[1,2-b]pyrazol-3-yl)-quinoline; 7-(3-Fluoro-propoxy)-4-(2-pyridin-2-yl-5,6-dihydro-4H-pyrrolo-[1,2-b]pyrazol-3-yl)-quinoline; 7-(3-Chloro-propoxy)-4-(2-pyridin-2-yl-5,6-dihydro-4H-pyrrolo[1,2-b]pyrazol-3-yl)-quinoline; 7-(3-Chloro-propoxy)-6-methoxy-4-(2-pyridin-2-yl-5,6-dihydro-4H-pyrrolo[1,2-b]pyrazol-3-yl)-quinoline; 7-(3-Chloro-propoxy)-4-[2-(6-methyl-pyridin-2-yl)-5,6-dihydro-4H-pyrrolo[1,2-b]pyrazol-3-yl]-quinoline; (1-{3-[7-(2-Chloro-ethoxy)-quinolin-4-yl]-5,6-dihydro-4H-pyrrolo[1,2-b]pyrazol-2-yl}-propenyl)-methylene-amine; N,N-Diethyl-2-[4-(2-pyridin-2-yl-5,6-dihydro-4-pyrrolo[1,2-b]pyrazol-3-yl)-quinolin-7-yloxy]-acetamide; 7-[2-((2R)-1-Methyl-pyrrolidin-2-yl)-ethoxy]-4-(2-pyridin-2-yl-5,6-dihydro-4H-pyrrolo[1,2-b]pyrazol-3-yl)-quinoline; Dimethyl-{4-[4-(2-pyridin-2-yl-5,6-dihydro-4H-pyrrolo[1,2-b]pyrazol-3-yl)-pyridin-2-yloxy]-butyl}-amine; 1-{3-[4-(2-Pyridin-2-yl-5,6-dihydro-4H-pyrrolo[1,2-b]pyrazol-3-yl)-pyridin-2-yloxy]-propyl}-pyrrolidin-2-one; 7-(1-Methyl-piperidin-3-ylmethoxy)-4-(2-pyridin-2-yl-5,6-dihydro-4H-pyrrolo[1,2-b]pyrazol-3-yl)-quinoline; 7-(3-N,N-Dimethylamino-2-methyl-propyloxy)-4-(2-pyridin-2-yl-5,6-dihydro-4H-pyrrolo[1,2-b]pyrazol-3-yl)-quinoline; 4-[2-(6-Methyl-pyridin-2-yl)-5,6-dihydro-4H-pyrrolo[1,2-b]pyrazol-3-yl]-7-propoxy-quinoline; 4-[6-Benzyloxymethyl-2-(6-methyl-pyridin-2-yl)-5,6-dihydro-4H-pyrrolo[1,2-b]pyrazol-3-yl]-quinoline; {4-[2-(6-Methyl-pyridin-2-yl)-5,6-dihydro-4H-pyrrolo[1,2-b]pyrazol-3-yl]-quinolin-7-yloxy}-acetic acid methyl ester; 7-Isopropoxy-4-[2-(6-methyl-pyridin-2-yl)-5,6-dihydro-4H-pyrrolo[1,2-b]pyrazol-3-yl]-quinoline; 4-[2-(6-Methyl-pyridin-2-yl)-5,6-dihydro-4H-pyrrolo[1,2-b]pyrazol-3-yl]-7-(3-morpholin-4-yl-propoxy)-quinoline; 4-(6-Benzyloxymethyl-2-pyridin-2-yl-5,6-dihydro-4H-pyrrolo[1,2-b]pyrazol-6-yl)-quinoline; 7-Benzyloxy-2-Pyridin-2-yl-3-quinolin-4-yl-pyrazolo[1,5-a]piperidine; 2-[4-(2-pyridin-2-yl-5,6-dihydro-4H-pyrrolo[1,2-b]pyrazol-3-yl)-quinolin-7-yloxy]-acetamide; 7-(5-Phenyl-[1,2,4]oxadiazol-3-ylmethoxy)-4-(2-pyridin-2-yl-5,6-dihydro-4H-pyrrolo[1,2-b]pyrazol-3-yl)-quinoline; 7-(2,2-Difluoro-benzo[1, 3]dioxol-5-ylmethoxy)-4-(2-pyridin-2-yl-5,6-dihydro-4H-pyrrolo[1,2-b]pyrazol-3-yl)-quinoline; 7-[2-(259-1-Methyl-pyrrolidin-2-yl)-ethoxy]-4-(2-pyridin-2-yl-5,6-dihydro-4H-pyrrolo[1,2-b]pyrazol-3-yl)-quinoline; 5-[4-(2-Pyridin-2-yl-5,6-dihydro-4H-pyrrolo[1,2-b]pyrazol-3-yl)-quinolin-7-yloxymethyl]-pyrrolidin-2-one; 4-(6-Phenoxymethyl-2-pyridin-2-yl-5,6-dihydro-4H-pyrrolo[1,2-b]pyrazol-3-yl)-quinoline; 4-(6-Methylene-2-pyridin-2-yl-5,6-dihydro-4H-pyrrolo[1,2-b]pyrazol-3-yl)-quinoline; 3-(4-Fluoro-phenyl)-6-methylene-2-(6-methyl-pyridin-2-yl)-5,6-dihydro-4H-pyrrolo[1,2-b]pyrazole; 7-(1-Methyl-piperidin-2-ylmethoxy)-4-(2-pyridin-2-yl-5,6-dihydro-4H-pyrrolo[1,2-b]pyrazol-3-yl)-quinoline hydrochloride; 7-[2-(1-Methyl-pyrrolidin-2-yl)-ethoxy]-4-(2-pyridin-2-yl-5,6-dihydro-4H-pyrrolo[1,2-b]pyrazol-3-yl)-quinoline hydrochloride; 4-[2-(6-Methyl-1-oxy-pyridin-2-yl)-5,6-dihydro-4H-pyrrolo[1,2-b]pyrazol-3-yl]-quinoline 1-oxide; 4-[2-(6-Methyl-pyridin-2-yl)-5,6-dihydro-4H-pyrrolo[1,2-b]pyrazol-3-yl]-quinoline 1-oxide; 4-[2-(6-Methyl-1-oxy-pyridin-2-yl)-5,6-dihydro-4H-pyrrolo[1,2-b]pyrazol-3-yl]-quinoline; 7-(3-Chloro-propoxy)-4-(2-pyridin-2-yl-5,6-dihydro-4H-pyrrolo[1,2-b]pyrazol-3-yl)-quinoline 1-oxide; 7-Methanesulfonyl-4-(2 pyridin-2-yl-5,6-dihydro-4H-pyrrolo[1,2-b]pyrazol-3-yl)-quinoline; 3-(4-Fluoro-phenyl)-2-(6-methyl-1-oxy-pyridin-2-yl)-5,6-dihydro-4H-pyrrolo[1,2-b]pyrazole; 4-(Quinolin-N-1-oxide-4-yl)-3-(6-methylpyridin-2-yl)-5,6-dihydro-4H-pyrrolo[1,2-b]pyrazole; 6-Methanesulfonyl-4-(2-pyridin-2-yl-5,6-dihydro-4H-pyrrolo[1,2-b]pyrazol-3-yl)-quinoline; 7-Ethanesulfonyl-4-(2-pyridin-2-yl-5,6-dihydro-4H-pyrrolo[1,2-b]pyrazol-3-yl)-quinoline; 4-(2-Pyridin-2-yl-5,6-dihydro-4H-pyrrolo[1,2-b]pyrazol-3-yl)-7-[3-(pyrimidine-2-sulfonyl)-propoxy]-quinoline; 7-[3-(1-Methyl-1H-imidazole-2-sulfonyl)-propoxy]-4-(2-pyridin-2-yl-5,6-dihydro-4H-pyrrolo[1,2-b]pyrazol-3-yl)-quinoline; 7-[3-(4-Chloro-benzenesulfonyl)-propoxy]-4-(2-pyridin-2-yl-5,6-dihydro-4H-pyrrolo[1,2-b]pyrazol-3-yl)-quinoline; 4-(2-Pyridin-2-yl-5,6-dihydro-4H-pyrrolo[1,2-b]pyrazol-3-yl)-7-[3-(pyridin-2-ylmethanesulfonyl)-propoxy]-quinoline; 4-(2-Pyridin-2-yl-5,6-dihydro-4H-pyrrolo[1,2-b]pyrazol-3-yl)-7-[3-pyridin-2-ylmethanesulfinyl)-propoxy]-quinoline; 4-(Quinolin-1-N-oxide-4-yl)-3-(6-methylpyridin-2-yl-1-N-oxide)-5,6-dihydro-4H-pyrrolo[1,2-b]pyrazole; 3-{4-[2-(6-Methyl-pyridin-2-yl)-5,6-dihydro-4H-pyrrolo[1,2-b]pyrazol-3-yl]-quinolin-7-yl}-acrylic acid methyl ester; 3-{4-[2-(6-Methylpyridin-2-yl-5,6-dihydro-4H-pyrrolo[1,2-b]pyrazol-3-yl]quinolin-7-yl}-1-piperidin-1-yl-propenone; 3-{4-[2-(6-Methyl-pyridin-2-yl)-5,6-dihydro-4H-pyrrolo[1,2-b]pyrazol-3-yl]-quinolin-6-yl}-acrylic acid methyl ester; 4-[2-(6-Methyl-pyridin-2-yl)-5,6-dihydro-4H-pyrrolo[1,2-b]pyrazol-3-yl]-7-vinyl-quinoline; 4-[2-(6-Benzyl-pyridin-2-yl)-5,6-dihydro-4H-pyrrolo[1,2-b]pyrazol-3-yl]-quinoline; 7-Benzyl-4-[2-(6-methyl-pyridin-2-yl)-5,6-dihydro-4H-pyrrolo[1,2-b]pyrazol-3-yl]-quinoline; 4-[2-(6-Methyl-pyridin-2-yl)-5,6-dihydro-4H-pyrrolo[1,2-b]pyrazol-3-yl]-quinoline-7-carboxylic acid; 4-[2-(6-Methyl-pyridin-2-yl)-5,6-dihydro-4H-pyrrolo[1,2-b]pyrazol-3-yl]-quinoline-6-carboxylic acid; 3-{4-[2-(6-Methy-pyridin-2-yl)-5,6-dihydro-4H-pyrrolo[1,2-b]pyrazol-3-yl]-quinolin-7-yl}-acrylic acid; 3-{4-[2-(6-Methyl-pyridin-2-yl)-5,6-dihydro-4H-pyrrolo[1,2-b]pyrazol-3-yl]-quinolin-7-yl}-propionic acid; 4-[2-(6-Methyl-pyridin-2-yl)-3-quinolin-4-yl-5,6-dihydro-4H-pyrrolo[1,2-b]pyrazol-5-yl]-benzoic acid; 4-(2-Pyridin-2-yl-5,6-dihydro-4H-pyrrolo[1,2-b]pyrazol-3-yl)-quinoline-7-carboxylic acid cyclopentylamide; 4-(2-Pyridin-2-yl-5,6-dihydro-4H-pyrrolo[1,2-b]pyrazol-3-yl)-quinoline-7-carboxylic acid (2-morpholin-4-yl-ethyl)-amide; 4-(2-Pyridin-2-yl-5,6-dihydro-4H-pyrrolo[1,2-b]pyrazol-3-yl)-quinoline-7-carboxylic acid [2-(1H-imidazol-4-yl)-ethyl]-amide; 4-(2-Pyridin-2-yl-5,6-dihydro-4H-pyrrolo[1,2-b]pyrazol-3-yl)-quinoline-7-carboxylic acid (2-methylamino-ethyl)-amide; 4-(2-Pyridin-2-yl-5,6-dihydro-4H-pyrrolo[1,2-b]pyrazol-3-yl)-quinoline-7-carboxylic acid (3-methylamino-propyl)-amide; 4-(2-Pyridin-2-yl-5,6-dihydro-4H-pyrrolo[1,2-b]pyrazol-3-yl)-quinoline-7-carboxylic acid (2-dimethylamino-ethyl)-amide; (4-Methyl-piperazin-1-yl)-[4-(2-pyridin-2-yl-5,6-dihydro-4H-pyrrolo[1,2-b]pyrazol-3-yl)-quinolin-7-yl]-methanone; 4-(2-Pyridin-2-yl-5,6-dihydro-4H-pyrrolo[1,2-b]pyrazol-3-yl)-quinoline-7-carboxylic acid cyclobutylamide; 4-(2-Pyridin-2-yl-5,6-dihydro-4H-pyrrolo[1,2-b]pyrazol-3-yl)-quinoline-7-carboxylic acid cyclopropylamide, 4-(2-Pyridin-2-yl-5,6-dihydro-4H-pyrrolo[1,2-b]pyrazol-3-yl)-quinoline-7-carboxylic acid (1-ethyl-propyl)-amide; 4-(2-Pyridin-2-yl-5,6-dihydro-4H-pyrrolo[1,2-b]pyrazol-3-yl)-quinoline-7-carboxylic acid ethylamide; 4-(2-Pyridin-2-yl-5,6-dihydro-4H-pyrrolo[1,2-b]pyrazol-3-yl)-quinoline-7-carboxylic acid isobutyl-amide; 4-(2-Pyridin-2-yl-5,6-dihydro-4H-pyrrolo[1,2-b]pyrazol-3-yl)-quinoline-7-carboxylic acid tert-butylamide; 4-(2-Pyridin-2-yl-5,6-dihydro-4H-pyrrolo[1,2-b]pyrazol-3-yl)-quinoline-7-carboxylic acid isopropylamide; 4-(2-Pyridin-2-yl-5,6-dihydro-4H-pyrrolo[1,2-b]pyrazol-3-yl)-quinoline-7-carboxylic acid propylamide; 4-(2-Pyridin-2-yl-5,6-dihydro-4H-pyrrolo[1,2-b]pyrazol-3-yl)-quinoline-7-carboxylic acid (2-methyl-butyl)-amide; 4-(2-Pyridin-2-yl-5,6-dihydro-4H-pyrrolo[1,2-b]pyrazol-3-yl)-quinoline-7-carboxylic acid ((2S)-2-methyl-butyl)-amide; 4-(2-Pyridin-2-yl-5,6-dihydro-4H-pyrrolo[1,2-b]pyrazol-3-yl)-quinoline-7-carboxylic acid (2S)-sec-butylamide; 4-(2-Pyridin-2-yl-5,6-dihydro-4H-pyrrolo[1,2-b]pyrazol-3-yl)-quinoline-7-carboxylic acid (2R)-sec-butylamide; 4-(2-Pyridin-2-yl-5,6-dihydro-4H-pyrrolo[1,2-b]pyrazol-3-yl)-quinoline-7-carboxylic acid ((IR)-1,2-dimethyl-propyl)-amide; 4-(2-Pyridin-2-yl-5,6-dihydro-4H-pyrrolo[1,2-b]pyrazol-3-yl)-quinoline-7-carboxylic acid (pyridin-4-ylmethyl)-amide; 4-(2-Pyridin-2-yl-5,6-dihydro-4H-pyrrolo[1,2-b]pyrazol-3-yl)-quinoline-7-carboxylic acid (pyridin-3-ylmethyl)-amide; 4-(2-Pyridin-2-yl-5,6-dihydro-4H-pyrrolo[1,2-b]pyrazol-3-yl)-quinoline-7-carboxylic acid (pyridin-2-ylmethyl)-amide; 6-(3-Quinolin-4-yl-5,6-dihydro-4H-pyrrolo[1,2-b]pyrazol-2-yl)-pyridine-2-carboxylic acid amide; 1-(4-Methyl-piperazin-1-yl)-2-[4-(2-pyridin-2-yl-5,6-dihydro-4H-pyrrolo[1,2-b]pyrazol-3-yl)-quinolin-7-yloxy]-ethanone; N-(2-dimethylamino-ethyl)-2-[4-(2-pyridin-2-yl-5,6-dihydro-4H-pyrrolo[1,2-b]pyrazol-3-yl)-quinolin-7-yloxy]-acetamide; N-(2-dimethylamino-ethyl)-N-methyl-2-[4-(2-pyridin-2-yl-5,6-dihydro-4H-pyrrolo[1,2-b]pyrazol-3-yl)-quinolin-7-yloxy]-acetamide; N,N-Dimethyl-3-[4-(2-pyridin-2-yl-5,6-dihydro-4H-pyrrolo[1,2-b]pyrazol-3-yl)-quinolin-7-yloxy]-benzamide; 4-(2-Pyridin-2-yl-5,6-dihydro-4H-pyrrolo[1,2-b]pyrazol-3-yl)-quinoline-7-carboxylic acid amide; 4-(2-Pyridin-2-yl-5,6-dihydro-4H-pyrrolo[1,2-H]pyrazol-3-yl)-quinoline-7-carboxylic acid (2-dimethylamino-ethyl)-methyl-amide; 4-(2-Pyridin-2-yl-5,6-dihydro-4H-pyrrolo[1,2-H]pyrazol-3-yl)-quinoline-7-carboxylic acid (3-dimethylamino-propyl)-methyl-amide; 4-(2-Pyridin-2-yl-5,6-dihydro-4H-pyrrolo[1,2-H]pyrazol-3-yl)-quinoline-7-carboxylic acid dimethylamide; 4-(2-Pyridin-2-yl-5,6-dihydro-4H-pyrrolo[1,2-H]pyrazol-3-yl)-quinoline-7-carboxylic acid methylamide; 4-(2-Pyridin-2-yl-5,6-dihydro-4H-pyrrolo[1,2-b]pyrazol-3-yl)-quinoline-7-carboxylic acid pyridin-2-ylamide; N-(2,2-Dimethylamino-ethyl)-N-methyl-3-{4-[2-(6-methyl-pyridin-2-yl)-5,6-dihydro-4H-pyrrolo[1,2-b]pyrazol-3-yl]-quinolin-7-yl}-propionamide; 2-(6-Methyl-pyridin-2-yl)-5,6-dihydro-4H-pyrrolo[1,2-b]pyrazol-3-yl]-quinoline-6-carboxylic acid (2-dimethylamino-ethyl)-amide; 4-[2-(6-Methyl-pyridin-2-yl)-5,6-dihydro-4H-pyrrolo[1,2-b]pyrazol-3-yl]-quinoline-6-carboxylic acid (3-dimethylamino-propyl)-amide; 4-[2-(6-Methyl-pyridin-2-yl)-5,6-dihydro-4H-pyrrolo[1,2-b]pyrazol-3-yl]-quinoline-6-carboxylic acid (2-morpholin-4-yl-ethyl)-amide; 1-[2-(Quinolin-4-yl)-1-(6-methyl-pyridin-2-yl)-5,6-dihydro-4H-pyrrolo[1,2-b]pyrazol-3-yl]quinoline-7-carboxylic acid N,N-dimethylaminoethylamide; 4-[2-(6-Methylpyridin-2-yl)-5,6-dihydro-4H-pyrrolo[1,2-b]pyrazol-3-yl]quinoline-7-carboxylic acid (2-piperidin-1-yl-ethyl)amide; N-(2-Dimethylamino-ethyl)-3-{4-[2-(6-methyl-pyridin-2-yl)-5,6-dihydro-4H-pyrrolo[1,2-b]pyrazol-3-yl]-quinolin-7-yl}-propionamide; 4-[2-(6-Methyl-pyridin-2-yl)-5,6-dihydro-4H-pyrrolo[1,2-b]pyrazol-3-yl]-quinoline-7-carboxylic acid (3-dimethylamino-propyl)-amide; 4-(2-(6-Methyl-pyridin-2-yl)-5,6-dihydro-4H-pyrrolo[1,2-b]pyrazol-3-yl]-quinoline-7-carboxylic acid (3-pyrrolidin-1-yl-propyl)-amide; 4-(2-(6-Methyl-pyridin-2-yl)-5,6-dihydro-4H-pyrrolo[1,2-b]pyrazol-3-yl]-quinoline-7-carboxylic acid (3-morpholin-4-yl-propyl)-amide; 3-{4-[2-(6-Methyl-pyridin-2-yl)-5,6-dihydro-4H-pyrrolo[1,2-b]pyrazol-3-yl]-quinolin-7-yl}-propionamide; 4-(2-Pyridin-2-yl-5,6-dihydro-4H-pyrrolo[1,2-b]pyrazol-3-yl)-quinoline-6-carboxylic acid (2-dimethylamino-ethyl)-amide; 4-(2-Pyridin-2-yl-5,6-dihydro-4H-pyrrolo[1,2-b]pyrazol-3-yl)-quinoline-6-carboxylic acid (2-morpholin-4-yl-ethyl)-amide; 4-(2-Pyridin-2-yl-5,6-dihydro-4H-pyrrolo[1,2-b]pyrazol-3-yl)-quinoline-6-carboxylic acid; 4-(2-Pyridin-2-yl-5,6-dihydro-4H-pyrrolo[1,2-b]pyrazol-3-yl)-quinoline-6-carboxylic acid hydrazide; 4-(2-Pyridin-2-yl-5,6-dihydro-4H-pyrrolo[1,2-b]pyrazol-3-yl)-quinoline-6-carboxylic acid amide; 4-(2-Pyridin-2-yl-5,6-dihydro-4H-pyrrolo[1,2-b]pyrazol-3-yl)-quinoline-6-carboxylic acid (3-methylamino-propyl)-amide; 4-(2-Pyridin-2-yl-5,6-dihydro-4H-pyrrolo[1,2-b]pyrazol-3-yl)-quinoline-6-carboxylic acid amide; 4-(2-Pyridin-2-yl-5,6-dihydro-4H-pyrrolo[1,2-b]pyrazol-3-yl)-quinoline-6-carboxylic acid (2-hydroxy-ethyl)-amide; 4-(2-Pyridin-2-yl-5,6-dihydro-4H-pyrrolo[1,2-b]pyrazol-3-yl)-quinoline-7-carboxylic acid hydrazide; 4-(2-Pyridin-2-yl-5,6-dihydro-4H-pyrrolo[1,2-b]pyrazol-3-yl)-quinoline-7-carboxylic acid hydroxyamide; 4-(2-Pyridin-2-yl-5,6-dihydro-4H-pyrrolo[1,2-b]pyrazol-3-yl)-quinoline-7-carboxylic acid (2-amino-ethyl)-amide; 4-(2-Pyridin-2-yl-5,6-dihydro-4H-pyrrolo[1,2-b]pyrazol-3-yl)-quinoline-7-carboxylic acid (2-hydroxy-ethyl)-amide; 4-(2-Pyridin-2-yl-5,6-dihydro-4H-pyrrolo[1,2-b]pyrazol-3-yl)-quinoline-7-sulfonic acid amide; 4-(2-Pyridin-2-yl-5,6-dihydro-4H-pyrrolo[1,2-b]pyrazol-3-yl)-quinoline-7-sulfonic acid methylamide; 4-(2-Pyridin-2-yl-5,6-dihydro-4H-pyrrolo[1,2-b]pyrazol-3-yl)-quinoline-7-sulfonic acid dimethylamide; 4-(2-Pyridin-2-yl-5,6-dihydro-4H-pyrrolo[1,2-b]pyrazol-3-yl)-quinoline-7-sulfonic acid (3-dimethylamino-propyl)-amide; 4-(2-Pyridin-2-yl-5,6-dihydro-4H-pyrrolo[1,2-b]pyrazol-3-yl)-quinoline-7-sulfonic acid diethylamide; 4-(2-Pyridin-2-yl-5,6-dihydro-4H-pyrrolo[1,2-b]pyrazol-3-yl)-quinoline-7-sulfonic acid (2-piperidin-1-yl-ethyl)-amide; 4-(2-Pyridin-2-yl-5,6-dihydro-4H-pyrrolo[1,2-b]pyrazol-3-yl)-quinoline-7-sulfonic acid (2-hydroxy-ethyl)-amide; 4-(2-Pyridin-2-yl-5,6-dihydro-4H-pyrrolo[1,2-b]pyrazol-3-yl)quinolin-7-ylamine; 2-Dimethylamino-N-[4-(2-pyridin-2-yl-5,6-dihydro-4H-pyrrolo[1,2-b]pyrazol-3-yl)-quinolin-7-yl]acetamide; 3-Dimethylamino-N-[4-(2-pyridin-2-yl-5,6-dihydro-4H-pyrrolo[1,2-b]pyrazol-3-yl)-quinolin-7-yl]propionamide; N-[4-(2-Pyridin-2-yl-5,6-dihydro-4H-pyrrolo[1,2-b]pyrazol-3-yl)-quinolin-7-yl]-methanesulfonamide; N-4-(2-Pyridin-2-yl-5,6-dihydro-4H-pyrrolo[1,2-b]pyrazol-3-yl)-quinolin-7-yl]-acetamide; 4-(2-Pyridin-2-yl-5,6-dihydro-4H-pyrrolo[1,2-b]pyrazol-3-yl)-quinoline-7-carboxylic acid (2-acetylamino-ethyl)-amide; N-{3-[4-(2-Pyridin-2-yl-5,6-dihydro-4H-pyrrolo[1,2-b]pyrazol-3-yl)-quinolin-7-yloxy]-propyl}-methanesulfonamide; 1-methyl-1H-imidazole-4-sulfonic acid {3-[4-(2-pyridin-2-yl-5,6-dihydro-4H-pyrrolo[1,2-b]pyrazol-3-yl)-quinolin-7-yloxy]-propyl}-amide; 1-(2-Dimethylamino-ethyl)-3-[4-(2-pyridin-2-yl-5,6-dihydro-4H-pyrrolo[1,2-b]pyrazol-3-yl)-quinolin-7-yl]-urea; 1-(3-Dimethylamino-propyl)-3-[4-(2-pyridin-2-yl-5,6-dihydro-4H-pyrrolo[1,2-b]pyrazol-3-yl)-quinolin-7-yl]-urea; 1-(2-Hydroxy-ethyl)-3-[4-(2-pyridin-2-yl-5,6-dihydro-4H-pyrrolo[1,2-b]pyrazol-3-yl)-quinolin-7-yl]-urea; [4-(2-Pyridin-2-yl-5,6-dihydro-4H-pyrrolo[1,2-b]pyrazol-3-yl)-quinolin-7-yl]-carbamic acid methyl ester; [4-(2-Pyridin-2-yl-5,6-dihydro-4H-pyrrolo[1,2-b]pyrazol-3-yl)-quinolin-7-yl]-carbamic acid 2-hydroxy-ethyl ester; [4-(2-Pyridin-2-yl-5,6-dihydro-4H-pyrrolo[1,2-b]pyrazol-3-yl)-quinolin-7-yl]-carbamic acid 2-methoxy-ethyl ester; 1,3-Bis-[4-(2-pyridin-2-yl-5,6-dihydro-4H-pyrrolo[1,2-b]pyrazol-3-yl)-quinolin-7-yl]-urea; Dimethyl-carbamic acid 4-(2-pyridin-2-yl-5,6-dihydro-4H-pyrrolo[1,2-b]pyrazol-3-yl)-quinolin-7-yl ester; 7-Bromo-2-isopropyl-4-(2-pyridin-2-yl-5,6-dihydro-4H-pyrrolo[1,2-b]pyrazol-3-yl)-quinoline; 2-[4-(2-(6-Methyl-pyridin-2-yl-5,6-dihydro-4H-pyrrolo[1,2-b]pyrazol-3-yl]-quinolin-6-yl)-propan-2-ol; 7-(3-Chloro-propylsulfanyl)-4-(2-pyridin-2-yl-5,6-dihydro-4H-pyrrolo[1,2-b]pyrazol-3-yl)-quinoline; 7-Bromo-4-(4-chloro-2-pyridin-2-yl-5,6-dihydro-4H-pyrrolo[1,2-b]pyrazol-3-yl)-quinoline; 8-Chloro-4-(2-Pyridin-2-yl-5,6-dihydro-4H-pyrrolo[1,2-b]pyrazol-3-yl)-quinolin-7-ol; 8-Bromo-4-(2-pyridin-2-yl-5,6-dihydro-4H-pyrrolo[1,2-b]pyrazol-3-yl)-quinolin-7-ol; 3-(7-Bromo-quinolin-4-yl)-2-pyridin-2-yl-5,6-dihydro-4H-pyrrolo[1,2-b]pyrazol-4-ol; 7-Bromo-4-(4-methoxy-2-pyridin-2-yl-5,6-dihydro-4H-pyrrolo[1,2-b]pyrazol-3-yl)-quinoline; [3-(7-Bromo-quinolin-4-yl)-2-pyridin-2-yl-5,6-dihydro-4H-pyrrolo[1,2-b]pyrazol-4-yl]-methyl-amine; 3-(7-Bromo-quinolin-4-yl)-2-pyridin-2-yl-5,6-dihydro-pyrrolo[1,2-b]pyrazol-4-one; 3-[4-(2-Pyridin-2-yl-5,6-dihydro-4H-pyrrolo[1,2-b]pyrazol-3-yl)-quinolin-7-yloxy]-benzamide; N,N-Dimethyl-3-[4-(2-pyridin-2-yl-5,6-dihydro-4H-pyrrolo[1,2-b]pyrazol-3-yl)-quinolin-7-yloxy]-thiobenzamide; Dimethyl-{3-[4-(2-Pyridin-2-yl-5,6-dihydro-4H-pyrrolo[1,2-b]pyrazol-3-yl)-quinolin-7-yloxy]-benzyl}-amine; 4-(2-(6-Methyl-pyridin-2-yl-5,6-dihydro-4H-pyrrolo[1,2-b]pyrazol-3-yl]-1H-quinolin-2-one; 4-(2-Pyridin-2-yl-5,6-dihydro-4H-pyrrolo[1,2-b]pyrazol-3-yl)-quinolin-7-ol; 4-[2-(6-Methyl-pyridin-2-yl)-5,6-dihydro-4H-pyrrolo[1,2-b]pyrazol-3-yl]-quinolin-7-ol; 6-Methoxy-4-(2-pyridin-2-yl-5,6-dihydro-4H-pyrrolo[1,2-b]pyrazol-3-yl)-quinolin-7-ol; 3-{4-[2-(6-Methyl-pyridin-2-yl)-5,6-dihydro-4H-pyrrolo[1,2-b]pyrazol-3-yl]-quinolin-7-yl}-propionic acid methyl ester; 4-(6-(Methyl-2-pyridin-2-yl-5,6-dihydro-4H-pyrrolo[1,2-b]pyrazol-3-yl)-quinoline; 3-{4-[2-(6-Methyl-pyridin-2-yl)-5,6-dihydro-4H-pyrrolo[1,2-b]pyrazol-3-yl]-quinolin-6-yl}-propionic acid methyl ester; 7-Amino-4-[2-(6-Methyl-pyridin-2-yl)-5,6-dihydro-4H-pyrrolo[1,2-b]pyrazol-3-yl]-quinoline; N,N-Dimethyl-3-{4-(2-methyl-pyridin-2-yl-5,6-dihydro-4H-pyrrolo[1,2-b]pyrazol-3-yl]-quinolin-7-}-propionamide; N-{3-[4-(2-Pyridin-2-yl-5,6-dihydro-4H-pyrrolo[1,2-b]pyrazol-3-yl)-quinolin-7-yloxy]-propyl}-acetamide; N-Acetyl-N-{4-[2-(6-methyl-pyridin-2-yl)-5,6-dihydro-4H-pyrrolo[1,2-b]pyrazol-3-yl]-quinolin-7-yl}-acetamide, 2-Pyridin-2-yl-3-quinolin-4-yl-pyrazolo[1,5-a]piperidin-7-ol; 7-Acetoxy-2-pyridin-2-yl-3-quinolin-4-yl-pyrazolo[1,5-a]piperidine; Methyl-{3-[4-(2-pyridin-2-yl-5,6-dihydro-4H-pyrrolo[1,2-b]pyrazol-3-yl)-quinolin-7-yloxy]-propyl}-amine; 7-(Piperidin-4-yloxy)-4-(2-pyridin-2-yl-5,6-dihydro-4H-pyrrolo[1,2-b]pyrazol-3-yl)-quinoline; 4-(6-(Methyl-2-pyridin-2-yl)-5,6-dihydro-4H-pyrrolo[1,2-b]pyrazol-3-yl]-quinoline-7-carboxylic acid (2-amino-1,1-dimethyl-ethyl)-amide; 16-[3-(4-Fluoro-phenyl)-5,6-dihydro-4H-pyrrolo[1,2-b]pyrazol-2-yl]pyridin-2-yl}-methanol, rrrrrm-rrrrr)[6-(3-Quinolin-4-yl-5,6-dihydro-4H-pyrrolo[1,2-b]pyrazol-2-yl)-pyridin-2-yl]methanol; 4-(6-(Methyl-2-pyridin-2-yl-5,6-dihydro-4H-pyrrolo[1,2-b]pyrazol-3-yl]-phenol; 7-(1-Methyl-pyrrolidin-3-ylmethoxy)-4-(2-pyridin-2-yl-5,6-dihydro-4H-pyrrolo[1,2-b]pyrazol-3-yl)-quinoline; 7-(1-Methyl-piperidin-4-ylmethoxy)-4-(2-pyridin-2-yl-5,6-dihydro-4H-pyrrolo[1,2-b]pyrazol-3-yl)-quinoline; 4-[2-(6-Methyl-pyridin-2-yl)-5,6-dihydro-4H-pyrrolo[1,2-b]pyrazol-3-yl]-quinoline-7-carboxylic acid (2-dimethylamino-1,1-dimethyl-ethyl)-amide; (S)-[3-(4-Fluoro-phenyl)-2-(6-methyl-pyridin-2-yl)-5,6-dihydro-4H-pyrrolo[1,2-b]pyrazol-6-yl]-methanol; (R)-[3-(4-Fluoro-phenyl)-2-(6-methyl-pyridin-2-yl)-5,6-dihydro-4H-pyrrolo[1,2-b]pyrazol-6-yl]-methanol; (S)-[3-(4-Fluoro-phenyl)-2-(6-methyl-pyridin-2-yl)-5,6-dihydro-4H-pyrrolo[1,2-b]pyrazol-6-yl]-acetonitrile; (R)-[3-(4-Fluoro-phenyl)-2-(6-methyl-pyridin-2-yl)-5,6-dihydro-4H-pyrrolo[1,2-b]pyrazol-6-yl]-acetonitrile; 4-(3-Pyridin-2-yl-5,6-dihydro-4H-pyrrolo[1,2-b]pyrazol-2-yl)-quinoline; 4-(6-Pyridin-2-yl-2,3-dihydro-pyrazolo[5,1-b]oxazol-7-yl)-quinoline; 3-[4-(2-Pyridin-2-yl-5,6-dihydro-4H-pyrrolo[1,2-b]pyrazol-3-yl)-quinolin-7-yl]-oxazolidin-2-one; 1-[4-(2-Pyridin-2-yl-5,6-dihydro-4H-pyrrolo[1,2-b]pyrazol-3-yl)-quinolin-7-yl]-imidazolidin-2-one; 4-(2-Pyridin-2-yl-5,6-dihydro-4H-pyrrolo[1,2-b]pyrazol-3-yl)-7-(pyridin-4-ylmethoxy)-quinoline; 4-(2-Pyridin-2-yl-5,6-dihydro-4H-pyrrolo[1,2-b]pyrazol-3-yl)-7-(3-pyridin-3-yl-propoxy)-quinoline; 7-(4,5-Dihydro-1H-imidazol-2-yl)-4-(2-pyridin-2-yl-5,6-dihydro-4H-pyrrolo[1,2-b]pyrazol-3-yl)-quinoline; 4-[5-(4-Fluoro-phenyl)-2-(6-methyl-pyridin-2-yl)-5,6-dihydro-4H-pyrrolo[1,2-b]pyrazol-3-yl]-quinoline (Enantiomer A); 4-[5-(4-Fluoro-phenyl)-2-(6-methyl-pyridin-2-yl)-5,6-dihydro-4H-pyrrolo[1,2-b]pyrazol-3-yl]-quinoline (Enantiomer B); 2-Pyridin-2-yl-3-quinolin-4-yl-pyrazolo[5,1-c]morpholine; 4-[2-(6-Vinyl-pyridin-2-yl)-5,6-dihydro-4H-pyrrolo[1,2-b]pyrazol-3-yl]-quinoline; 3-{4-[2-(6-Methyl-pyridin-2-yl)-5,6-dihydro-4H-pyrrolo[1,2-b]pyrazol-3-yl]-quinolin-6-yl}-acrylic acid; 7-(6-Methyl-pyridin-3-yloxy)-4-(2-pyridin-2-yl-5,6-dihydro-4H-pyrrolo[1,2-b]pyrazol-3-yl)-quinoline; 4-(2-Pyridin-2-yl-5,6-dihydro-4H-pyrrolo[1,2-b]pyrazol-3-yl)-7-[4-(4-pyrimidin-2-yl-piperazin-1-yl)-butoxy]-quinoline; 7-[3-[4-(2-Methoxy-phenyl)-piperazin-1-yl]-propoxy]-4-(2-pyridin-2-yl-5,6-dihydro-4H-pyrrolo[1,2-b]pyrazol-3-yl)-quinoline; Pyridin-2-yl-{3-[4-(2-pyridin-2-yl-5,6-dihydro-4H-pyrrolo[1,2-b]pyrazol-3-yl)-quinolin-7-yloxy]-propyl}-amine; 4-(6-(Methyl-pyridin-2-yl)-5,6-dihydro-4H-pyrrolo[1,2-b]pyrazol-3-yl]-quinoline-7-carboxylic acid (2-dimethylamino-1-methyl-ethyl)-amide, rrrrnTn-rr)4-[2-(6-Methyl-pyridin-2-yl)-5,6-dihydro-4H-pyrrolo[1,2-b]pyrazol-3-yl]-quinoline-7-carboxylic acid amide; 4-(2-Pyridin-2-yl-5,6-dihydro-4H-pyrrolo[1,2-b]pyrazol-3-yl)-quinoline-7-carboxylic acid (3-dimethylamino-propyl)-amide; 4-[2-(6-Methyl-pyridin-2-yl)-5,6-dihydro-4H-pyrrolo[1,2-b]pyrazol-3-yl]-quinoline-7-carboxylic acid (2-dimethylamino-ethyl)-methyl-amide; N,N-Dimethyl-3-{4-[2-(6-methyl-pyridin-2-yl)-5,6-dihydro-4H-pyrrolo[1,2-b]pyrazol-3-yl]-quinolin-7-yl}-acrylamide; 4-(2-Pyridin-2-yl-5,6-dihydro-4H-pyrrolo[1,2-b]pyrazol-3-yl)-quinoline 1-oxide; 7-Benzyloxy-4-[2-(6-methyl-pyridin-2-yl)-5,6-dihydro-4H-pyrrolo[1,2-b]pyrazol-3-yl]-quinoline; 4-(2-(6-Chloro-6-dihydro-4H-pyrrolo-pyridin-2-yl)-5[1,2-b]pyrazol-3-yl)-quinoline; 6-(3-Quinolin-4-yl-5,6-dihydro-4H-pyrrolo[1,2-b]pyrazol-2-yl)pyridine-2-carboxylic acid methyl ester; 4-(7-Chloroquinolin-4-yl)-3-(pyridin-2-yl)-5,6-dihydro-4H-pyrrolo[1,2-b]pyrazole; 4-(2-Furan-2-yl-5,6-dihydro-4H-pyrrolo[1,2-b]pyrazol-3-yl)-quinoline; 3-{4-(6-Methyl-pyridin-2-yl)-5,6-dihydro-4H-pyrrolo[1,2-b]pyrazol-3-yl]-quinolin-6-yl}-acrylic acid methyl ester; 4-[2-(2-Methyl-thiazol-4-yl)-5,6-dihydro-4H-pyrrolo[1,2-b]pyrazol-3-yl]-quinoline; 3-(4-Fluoro-phenyl)-2-(2-methyl-thiazol-4-yl)-5,6-dihydro-4H-pyrrolo[1,2-b]pyrazole; 4-[2-(2-Methyl-2H-pyrazol-3-yl)-5,6-dihydro-4H-pyrrolo[1,2-b]pyrazol-3-yl]-quinoline; 4-(2-Thiazol-2-yl-5,6-dihydro-4H-pyrrolo[1,2-b]pyrazol-3-yl)-quinoline; 4-[2-(1-Methyl-1H-imidazol-2-yl-5,6-dihydro-4H-pyrrolo[1,2-b]pyrazol-3-yl]-quinoline; 6,7-Dichloro-4-[2-(6-methyl-pyridin-2-yl)-5,6-dihydro-4H-pyrrolo[1,2-b]pyrazol-3-yl]-quinoline; (S)-6-Benzyloxymethyl-3-(4-fluoro-phenyl)-2-(6-methyl-pyridin-2-yl)-5,6-dihydro-4H-pyrrolo[1,2-b]pyrazole; N,N-Dimethyl-3-{4-[2-(6-methyl-pyridin-2-yl)-5,6-dihydro-4H-pyrrolo[1,2-b]pyrazol-3-yl]-quinolin-7-yl}-acrylamide; 3-methyl-6-[2-[6-methyl-(pyridin-2-yl)]-5,6-di-hydro-4H-pyrrolo[1,2-b]pyrazol-3-yl]-3H-quinazolin-4-one; 1-methyl-7-[2-[6-methyl-(pyridin-2-yl)]-5,6-di-hydro-4H-pyrrolo[1,2-b]pyrazol-3-yl]-1 Hl-quinoxalin-2-one; 3-methyl-6-[2-(pyridin-2-yl)-5,6-dihydro-4H-pyrrolo[1,2-b]pyrazol-3-yl]-3H-quinazolin-4-one; 3-methyl-6-[2-[6-pentyl-(pyridin-2-yl)]-5,6-di-hydro-4H-pyrrolo[1,2-b]pyrazol-3-yl]-3H-quinazolin-4-one; 6-[2-[6-Methyl-(pyridin-2-yl)]-5,6-dihydro-4H-pyrrolo[1,2-b]pyrazol-3-yl]-4H-benzo[1,4]oxazin-3-one; 3-(2-Chloro-ethyl)-6-[2-[6-methyl-(pyridin-2-yl)]-5,6-dihydro-4H-pyrrolo[1,2-b]pyrazol-3-yl]-3H-quinazolin-4-one; 6-[2-[6-methyl-(pyridin-2-yl)]-5,6-dihydro-4H-pyrrolo[1,2-b]pyrazol-3-yl]-3-(2-morpholin-4-yl-ethyl)-3H-quinazolin-4-one; 3-(2-Dimethylamino-ethyl)-6-[2-[6-methyl-(pyridin-2-yl)]-5,6-dihydro-4H-pyrrolo[1,2-b]pyrazol-3-yl]-3H-quinazolin-4-one; 6-[2-[6-Methyl-(pyridin-2-yl)]-5,6-dihydro-4H-pyrrolo[1,2-b]pyrazol-3-yl]-3-(2-piperidin-1-yl-ethyl)-3H-quinazolin-4-one; 6-[2-[6-Methyl-(pyridin-2-yl)]-5,6-dihydro-4H-pyrrolo[1,2-b]pyrazol-3-yl]-3-(2-pyrrolidin-1-yl-ethyl)-3H-quinazolin-4-one; 3-(2-Azepan-1-yl-ethyl)-6-[2-[6-methyl-(pyridin-2-yl)]-5,6-dihydro-4H-pyrrolo[1,2-b]pyrazol-3-yl]-3H-quinazolin-4-one; 7-[2-[6-Methyl-(pyridin-2-yl)]-5,6-dihydro-4H-pyrrolo[1,2-b]pyrazol-3-yl]-1-(2-pyrrolidin-1-yl-ethyl)-3,4-dihydro-1H-quinoxalin-2-one; and 1-(2-Dimethylamino-ethyl)-7-[2-[6-methyl-(pyridin-2-yl)]-5,6-dihydro-4H-pyrrolo[1,2-b]pyrazol-3-yl]-3,4-dihydro-1H-quinoxalin-2-one.

It should be appreciated that contacting the non-neuronal cell (e.g., somatic cell) with an agent which inhibits the level or activity of ALK4, ALK5, and ALK7 can done at any time during the conversion of the non-neuronal cell (e.g., somatic cell) to neurons. In some embodiments, the contacting is done during at least one of from days 1 to 5, days 6 to 10, and days 11 to 15 of conversion of non-neuronal cells (e.g., somatic cell) to neurons.

It should be appreciated that contacting the non-neuronal cell (e.g., somatic cell) with an agent which inhibits the level or activity of ALK4, ALK5, and ALK7 can done at any time during the conversion of the non-neuronal cell (e.g., somatic cell) to motor neurons. In some embodiments, the contacting is done during at least one of from days 1 to 5, days 6 to 10, and days 11 to 15 of transdifferentiation of the somatic cells to motor neurons.

In some embodiments, an isolated population of iNs and compositions are produced by a method comprising contacting a cell or a population of a non-neuronal cell (e.g., somatic cell, e.g., fibroblast) with an agent, such as a nucleic acid agent, peptide, polypeptide aptamer, antibody, antibody fragment, ribosomes, small molecules, RNAi agents, ribosomes and the like, which inhibits the level of activity of PLK1 in the non-neuronal cell (e.g., somatic cell).

In some embodiments, an isolated population of iMNs and compositions are produced by a method comprising contacting a cell or a population of a non-neuronal cell (e.g., somatic cell, e.g., fibroblast) with an agent, such as a nucleic acid agent, peptide, polypeptide aptamer, antibody, antibody fragment, ribosomes, small molecules, RNAi agents, ribosomes and the like, which inhibits the level of activity of PLK1 in the non-neuronal cell (e.g., somatic cell).

In some embodiments, the level or activity of PLK1 is inhibited by contacting the non-neuronal cell (e.g., somatic cell) with an agent which decreases the level or activity of PLK1. Any agent can be used, as long as the agent decreases the level or activity of PLK1, for example, as measured by phosphorylation of a PLK1 substrate by PLK1. Exemplary agents include, but are not limited to, small organic or inorganic molecules; saccharines; oligosaccharides; polysaccharides; a biological macromolecule selected from the group consisting of antibodies, peptides, proteins, peptide analogs and derivatives, and dominant negative variants; peptidomimetics; nucleic acids selected from the group consisting of microRNAs, siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

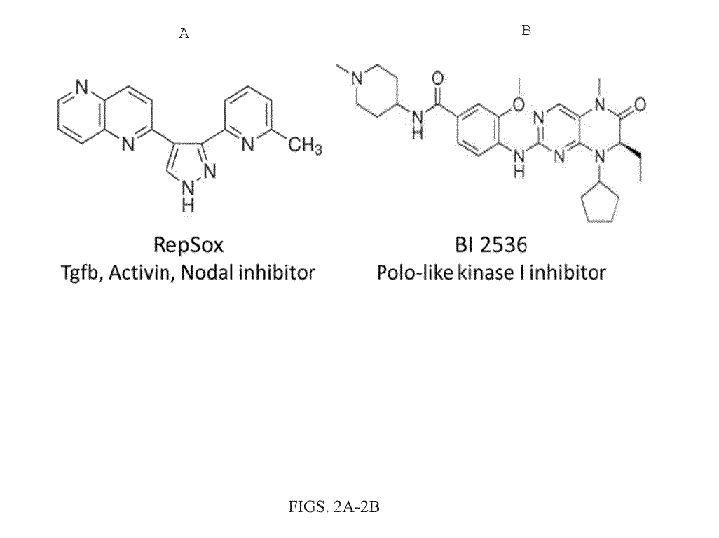

In some embodiments, the agent which inhibits the level or activity of PLK1 comprises methoxy-N-(1-methylpiperidin-4-yl)benzamide (BI 2536), the chemical structure of which is shown in FIG. 2B.

In some embodiments, the agent is an analog or derivative of BI 2536. In some embodiments, an analog or derivative of BI 2536 is a compound other than BI 2536 of formula (II):

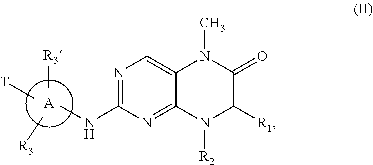

wherein R₁ is hydrogen, or an optionally substituted (C₁-C₆)alkyl, (C₂-C₆)alkenyl, (C₂-C₆)alkynyl or (C₃-C₆)cycloalkyl group; R₂ is hydrogen, or an optionally substituted (C₁-C₆)alkyl, (C₂-C₆)alkenyl, (C₂-C₆)alkynyl or (C₃-C₆)cycloalkyl group; R₃ and R₃′ are independently selected from hydrogen, —CN, hydroxyl, halogen, optionally substituted (C₁-C₆)alkyl, (C₂-C₆)alkenyl, (C₂-C₆)alkynyl or (C₃-C₆)cycloalkyl, —NR₅R₆ or C₁-C₄ alkoxy, wherein R₅ and R₆ are independently hydrogen or optionally substituted (C₁-C₆)alkyl; ring A is an optionally substituted mono- or bi-cyclic carbocyclic or heterocyclic ring or a ring system having up to 12 ring atoms; T is a radical of formula R-L¹-Y¹— wherein Y¹ is a bond, —O—, —S—, —NR₆—, —(C═O)—, —S(O₂)—, —(C═O)NR₆—, —NR₆(C═O)—, —S(O₂)NR₆—, —NR₆S(O₂)—, or —NR₆(C═O)NR₉—, wherein R₆ and R₉ are independently hydrogen or optionally substituted (C₁-C₆)alkyl; L¹ is a divalent radical of formula -(Alk¹)ₘ(Q)ₙ(Alk²)ₚ- wherein m, n and p are independently 0 or 1, Q is (i) an optionally substituted divalent mono- or bicyclic carbocyclic or heterocyclic radical having 5-13 ring members, or (ii), in the case where p is 0, a divalent radical of formula -Q¹-X²— wherein X² is —O—, —S— or NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members, Alk¹ and Alk² independently represent optionally substituted divalent (C₃-C₆)cycloalkyl radicals, or optionally substituted straight or branched, (C₁-C₆)alkylene, (C₂-C₆)alkenylene, or (C₂-C₆)alkynylene radicals which may optionally contain or terminate in an ether (—O—), thioether (—S—) or amino (—NR^{A}—) link wherein R^{A} is hydrogen or optionally substituted (C₁-C₃)alkyl; R is a radical of formula (X) or (Y) wherein R₇ is a carboxylic acid group (—COOH), or an ester group which is hydrolysable by one or more intracellular carboxylesterase enzymes to a carboxylic acid group; R₈ is hydrogen; or optionally substituted C₁-C₆ alkyl, C₃-C₇ cycloalkyl, aryl or heteroaryl or —(C═O)R₆, —(C═O)OR₆, or —(C═O)NR₆ wherein R₆ is hydrogen or optionally substituted (C₁-C₆)alkyl; and D is a monocyclic heterocyclic ring of 5 or 6 ring atoms wherein R₇ is linked to a ring carbon adjacent the ring nitrogen shown, and ring D is optionally fused to a second carbocyclic or heterocyclic ring of 5 or 6 ring atoms in which case the bond shown intersected by a wavy line may be from a ring atom in said second ring.

In some embodiments, the analog or derivative of BI 2536 is not 4-[[(7R)-8-cyclopentyl-7-ethyl-5-methyl-6-oxo-7H-pteridin-2-yl]amino]-3-methoxy-N-(1-methylpiperidin-4-yl)benzamide (BI 2536).

In some embodiments, the analog or derivative of BI 2536 is a pteridine derivative described in U.S. Patent Publication No. 2010/0216802, including for example, Cyclopentyl 4-[(4-{[(7R)-8-cyclopentyl-7-ethyl-5-methyl-6-oxo-5,6,7,8-tetrahydro pteridin-2-yl]amino}-3-methoxybenzoyl)amino]-phenylalaninate, Cyclopentyl O-(4-{[(4-{[(7R)-8-cyclopentyl-7-ethyl-5-methyl-6-oxo-5,6,7,8-tetrahydro pteridin-2-yl]amino}-3-methoxybenzoyl)amino]methyl}phenyl)-L-homoserinate, tert-butyl 4-[(4-{[(7R)-8-cyclopentyl-7-ethyl-5-methyl-6-oxo-5,6,7,8-tetrahydropteridin-2-yl]amino}-3-methoxybenzoyl)amino]-L-phenylalaninate, tert-Butyl O-(4-{[(4-{[(7R)-8-cyclopentyl-7-ethyl-5-methyl-6-oxo-5,6,7,8-tetrahydro pteridin-2-yl]amino}-3-methoxybenzoyl)amino]methyl}phenyl)-L-homoserinate, Cyclopentyl 4-{2-[(4-{[(7R)-8-cyclopentyl-7-ethyl-5-methyl-6-oxo-5,6,7,8-tetrahydro pteridin-2-yl]amino}-3-methoxybenzoyl)amino]ethyl}piperazine-2-carboxylate, tert-butyl 4-{2-[(4-{[(7R)-8-cyclopentyl-7-ethyl-5-methyl-6-oxo-5,6,7,8-tetrahydro pteridin-2-yl]amino}-3-methoxybenzoyl)amino]ethyl}piperazine-2-carboxylate, Cyclopentyl (2S)-2-amino-4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoate, tert-butyl 5-{4-[(4-{[(7R)-8-cyclopentyl-7-ethyl-5-methyl-6-oxo-5,6,7,8-tetrahydro pteridin-2-yl]amino}-3-methoxybenzoyl)amino]piperidin-1-yl}-L-norvalinate, Cyclopentyl 5-{4-[(4-{[(7R)-8-cyclo pentyl-7-ethyl-5-methyl-6-oxo-5,6,7,8-tetrahydro pteridin-2-yl]amino}-3-methoxybenzoyl)amino]piperidin-1-yl}-L-norvalinate, t-butyl (2S)-2-amino-4-{4-[(4-{[(7R)-8-cyclopentyl-7-ethyl-5-methyl-6-oxo-5,6,7,8-tetrahydropteridin-2-yl]amino}-3-methoxy benzoyl)amino]piperidin-1-yl}butanoate, t-butyl (2S)-2-amino-4-{4-[(4-{[(7R)-8-cyclopentyl-7-ethyl-5-methyl-6-oxo-5,6,7,8-tetrahydropteridin-2-yl]amino}-3-methylbenzoyl)amino]piperidin-1-yl}butanoate, Cyclopentyl (2S)-2-amino-4-{4-[(4-{[(7R)-8-cyclopentyl-7-ethyl-5-methyl-6-oxo-5,6,7,8-tetrahydropteridin-2-yl]amino}-3-methylbenzoyl)amino]piperidin-1-yl}butanoate, t-butyl (2S)-2-amino-4-{4-[(4-{[(7R)-8-cyclopentyl-7-ethyl-5-methyl-6-oxo-5,6,7,8-tetrahydropteridin-2-yl]amino}-3-fluorobenzoyl)amino]piperidin-1-yl}butanoate, Cyclopentyl (2S)-2-amino-4-{4-[(4-{[(7R)-8-cyclopentyl-7-ethyl-5-methyl-6-oxo-5,6,7,8-tetrahydropteridin-2-yl]amino}-3-fluorobenzoyl)amino]piperidin-1-yl}butanoate, and salts, N-oxides, hydrates or solvates thereof.

In some embodiments, the analog or derivative of BI 2536 comprises a hydrate or polymorph of 4[[(7R)-8-cyclopentyl-7-ethyl-5,6,7,8-tetrahydro-5-methyl-6-oxo-2-pteridi-nyl]amino]-3-methoxy-N-(1-methyl-4-piperidinyl)-benzamide, as described in U.S. Pat. No. 7,728,134, which is incorporated herein by reference.

In some embodiments, an isolated population of iNs and compositions are produced by a method comprising contacting a cell or a population of a non-neuronal cell (e.g., somatic cell, e.g., fibroblast) with an agent, such as a nucleic acid agent, peptide, polypeptide aptamer, antibody, antibody fragment, ribosomes, small molecules, RNAi agents, ribosomes and the like, which inhibits the level of activity of ALK4, ALK5, ALK7, and PLK1 in the non-neuronal cell (e.g., somatic cell).

In some embodiments, an isolated population of iMNs and compositions are produced by a method comprising contacting a cell or a population of a non-neuronal cell (e.g., somatic cell, e.g., fibroblast) with at least one agent, such as a nucleic acid agent, peptide, polypeptide aptamer, antibody, antibody fragment, ribosomes, small molecules, RNAi agents, ribosomes and the like, which inhibits the level of activity of ALK4, ALK5, ALK7, and PLK1 in the non-neuronal cell (e.g., somatic cell).

In some embodiments, an isolated population of iNs and compositions are produced by a method comprising increasing the levels of protein expression of at least one factor selected from the group consisting of Lhx3, Ascl1, Brn2, Myt1l, Isl1, Hb9, Ngn2 and NeuroD1; and contacting a cell or a population of a non-neuronal cell (e.g., somatic cell) with at least one agent, such as a nucleic acid agent, peptide, polypeptide aptamer, antibody, antibody fragment, ribosomes, small molecules, RNAi agents, ribosomes and the like, which inhibits the level of activity of ALK4, ALK5, ALK7, and PLK1 in the non-neuronal cell (e.g., somatic cell).

### DEFINITIONS

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “transdifferentiation,” “transdifferentiated,” and “transdifferentiating” are used interchangeably herein with the phrase “direct conversion” or “direct reprogramming” and refer to the conversion of one differentiated somatic cell type into a different differentiated somatic cell type without undergoing complete reprogramming to an induced pluripotent stem cell (iPSC) intermediate.

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. A partial reversal of differentiation produces a partially induced pluripotent (PiPS) cell. Reprogramming also encompasses partial reversion of the differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers.

The term “differentiated cell” is meant any primary cell that is not, in its native form, pluripotent as that term is defined herein. It should be noted that placing many primary cells in culture can lead to some loss of fully differentiated characteristics. However, simply culturing such cells does not, on its own, render them pluripotent. The transition to pluripotency requires a reprogramming stimulus beyond the stimuli that lead to partial loss of differentiated character in culture. Reprogrammed pluripotent cells also have the characteristic of the capacity of extended passaging without loss of growth potential, relative to primary cell parents, which generally have capacity for only a limited number of divisions in culture. Stated another way, the term “differentiated cell” refers to a cell of a more specialized cell type derived from a cell of a less specialized cell type (e.g., a stem cell such as an induced pluripotent stem cell) in a cellular differentiation process.

As used herein, the term “somatic cell” refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, which means a somatic cell that is not present in or obtained from an embryo and does not result from proliferation of such a cell in vitro. In some embodiments the somatic cell is an “adult somatic cell”, which means a cell that is present in or obtained from an organism other than an embryo or a fetus or results from proliferation of such a cell in vitro. Unless otherwise indicated the methods for direct conversion of a somatic cell, e.g., fibroblast to a iN or iMN can be performed both in vivo and in vitro (where in vivo is practiced when a somatic cell, e.g., fibroblast are present within a subject, and where in vitro is practiced using an isolated somatic cell, e.g., fibroblast maintained in culture).

As used herein, the term “adult cell” refers to a cell found throughout the body after embryonic development.

As used herein, the terms “iPS cell” and “induced pluripotent stem cell” are used interchangeably and refer to a pluripotent stem cell artificially derived (e.g., induced or by complete reversal) from a non-pluripotent cell, typically an adult somatic cell, for example, by inducing a forced expression of one or more genes.

The term “motor neuron” also referred to as a “motoneuron” refers to a neuron that sends electrical output signals to a muscle, gland, or other effector tissue.

The term “induced neuron” or “iN” as used herein refers to a functional neuron produced by direct conversion from a non-neuronal cell (from a less differentiated cell such as a stem cell or pluripotent cell or from an alternate cell type such as a non-neuronal somatic cell).

The term “induced motor neuron” or “iMN” as used herein refers to a functional motor neuron produced by direct conversion from a non-neuronal cell (from a less differentiated cell such as a stem cell or pluripotent cell or from an alternate cell type such as a non-neuronal somatic cell).

The term “functional” as used in relation to a neuron (e.g., motor neuron) refers to a motor neuron which can fire action potentials and can signal a muscle to contract. A functional motor neuron expresses ChAT, an enzyme necessary for synthesizing the motor neuron transmitter acetylcholine, and expresses VAChAT, which is necessary for the storage and uptake of the transmitter acetylcholine, and expresses synapsin for formation of synapses, and can transmit action potentials and synapse with muscle cells to result in muscle contraction.

As used herein, the term “endogenous motor neuron” refers to a motor neuron in vivo or a motor neuron produced by differentiation of an embryonic stem cell into a motor neuron, and exhibiting an adult motor neuron phenotype. The phenotype of a motor neuron is well known by persons of ordinary skill in the art, and include, for example, formation of synaptic junctions with muscle cells, expression of ChAT, immunostaining with aBTX, responsive to inhibitory and excitatory neurotransmitters, as well as distinct morphological characteristics such long axonal projections and synaptic connections with muscle cells.

The term “progenitor cell” is used herein to refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stemness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art.

In the context of cell ontogeny, the adjective “differentiated”, or “differentiating” is a relative term meaning a “differentiated cell” is a cell that has progressed further down the developmental pathway than the cell it is being compared with, Thus, stem cells can differentiate to lineage-restricted precursor cells (such as a mesodermal stem cell), which in turn can differentiate into other types of precursor cells further down the pathway (such as a cardiomyocyte precursor), and then to an end-stage differentiated cell, which plays a characteristic role in a certain tissue type, and may or may not retain the capacity to proliferate further.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue.

Accordingly, the disclosure appreciates that stem cell populations can be isolated from virtually any animal tissue.

The term a “MN-inducing factor”, as used herein, refers to a gene whose expression, contributes to the direct conversion of a somatic cell (e.g., fibroblast) to a MN which exhibits at least two characteristics of an endogenous motor neuron. A MN-inducing factor be, for example, genes encoding transcription factors Lhx3, Ascl1, Brn2, Myt1l, Isl1, Hb9, Ngn2 or NeuroD1, the sequences of which are disclosed in PCT International Application WO2013/025963, which are all incorporated herein by reference.

The term “MN-inducing agent” refers to any agent which increases the protein expression of a MN-inducing factor, as that term is described herein. Preferably, a MN-inducing agent increases the expression of a MN-inducing factor selected from Lhx3, Ascl1, Brn2, Myt1l, Isl1, Hb9, Ngn2 or NeuroD1.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The term “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. ScL USA 87:22264-2268, 1990) modified as in Karlin and Altschul, Proc. Natl. Acad. ScL USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL www.ncbi.nlm.nih.gov for these programs. In a specific embodiment, percent identity is calculated using BLAST2 with default parameters as provided by the NCBI.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of iNs, refers to a population of cells that contain fewer than about 20%, more preferably fewer than about 15%, 10%, 8%, 7%, most preferably fewer than about 5%, 4%, 3%, 2%, 1%, or less than 1%, of cells that are not iNs or their progeny as defined by the terms herein. In some embodiments, the disclosure encompasses methods to expand a population of iNs, wherein the expanded population of iNs is a substantially pure population of iNs. Recast, the terms “substantially pure” or “essentially purified”, with regard to a population of iMNs, refers to a population of cells that contain fewer than about 20%, more preferably fewer than about 15%, 10%, 8%, 7%, most preferably fewer than about 5%, 4%, 3%, 2%, 1%, or less than 1%, of cells that are not iMNs or their progeny as defined by the terms herein. In some embodiments, the disclosure encompasses methods to expand a population of iMNs, wherein the expanded population of iMNs is a substantially pure population of iMNs.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

As used herein, the term “DNA” is defined as deoxyribonucleic acid.

As used herein, the term “gene” used herein can be a genomic gene comprising transcriptional and/or translational regulatory sequences and/or a coding region and/or non-translated sequences (e.g., introns, 5′- and 3′-untranslated sequences and regulatory sequences). The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene can also be an amplified nucleic acid molecule produced in vitro comprising all or a part of the coding region and/or 5′- or 3′-untranslated sequences linked thereto.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxy thymidine, deoxy guanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated. The terms “nucleic acid” can also refer to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein. Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribonucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

A nucleic acid will generally contain phosphodiester bonds, although nucleic acid analogs can be included that can have at least one different linkage, e.g., phosphoramidate, phosphorothioate, phosphorodithioate, or O-methylphophoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated herein by reference. Nucleic acids containing one or more non-naturally occurring or modified nucleotides are also included within one definition of nucleic acids. The modified nucleotide analog can be located for example at the 5′-end and/or the 3′-end of the nucleic acid molecule. Representative examples of nucleotide analogs can be selected from sugar- or backbone-modified ribonucleotides. It should be noted, however, that also nucleobase-modified ribonucleotides, i.e. ribonucleotides, containing a non naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7 deaza-adenosine; O- and N-alkylated nucleotides, e.g. N6-methyl adenosine are suitable. The 2′ OH— group can be replaced by a group selected from H, OR, R, halo, SH, SR, NH2, NHR, NR2 or CN, wherein R is C—C6 alkyl, alkenyl or alkynyl and halo is F, Cl, Br or I. Modifications of the ribose-phosphate backbone can be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs can be made.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation.

Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The terms “polypeptide variant” refers to any polypeptide differing from a naturally occurring polypeptide by amino acid insertion(s), deletion(s), and/or substitution(s), Variants may be naturally occurring or created using, e.g., recombinant DNA techniques or chemical synthesis. In some embodiments amino acid “substitutions” are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Insertions or deletions may range in size from about 1 to 20 amino acids, e.g., 1 to 10 amino acids. In some instances larger domains may be removed without substantially affecting function. In certain embodiments of the invention the sequence of a variant can be obtained by making no more than a total of 5, 10, 15, or 20 amino acid additions, deletions, or substitutions to the sequence of a naturally occurring enzyme. In some embodiments not more than 1%, 5%, 10%, 15% or 20% of the amino acids in a polypeptide are insertions, deletions, or substitutions relative to the original polypeptide. Guidance in determining which amino acid residues may be replaced, added, or deleted without eliminating or substantially reducing activities of interest, may be obtained by comparing the sequence of the particular polypeptide with that of homologous polypeptides (e.g., from other organisms) and minimizing the number of amino acid sequence changes made in regions of high homology (conserved regions) or by replacing amino acids with those found in homologous sequences since amino acid residues that are conserved among various species are more likely to be important for activity than amino acids that are not conserved.

By “amino acid sequences substantially homologous” to a particular amino acid sequence (e.g. Lhx3, Ascl1, Brn2, Myt1l, Isl1, Hb9, Ngn2 or NeuroD1) is meant polypeptides that include one or more additional amino acids, deletions of amino acids, or substitutions in the amino acid sequence of Lhx3, Ascl1, Brn2, Myt1l, Isl1, Hb9, Ngn2 or NeuroD1 without appreciable loss of functional activity as compared to wild-type Lhx3, Ascl1, Brn2, Myt1l, Isl1, Hb9, Ngn2 or NeuroD1 polypeptides in terms of the ability to produce iMNs from a somatic cell, e.g., fibroblast. For example, the deletion can consist of amino acids that are not essential to the presently defined differentiating activity and the substitution(s) can be conservative (i.e., basic, hydrophilic, or hydrophobic amino acids substituted for the same). Thus, it is understood that, where desired, modifications and changes may be made in the amino acid sequence of Lhx3, Ascl1, Brn2, Myt1l, Isl1, Hb9, Ngn2 or NeuroD1, and a protein having like characteristics still obtained. It is thus contemplated that various changes may be made in the amino acid sequence of the Lhx3, Ascl1, Brn2, Myt1l, Isl1, Hb9, Ngn2 or NeuroD1 amino acid sequence (or underlying nucleic acid sequence) without appreciable loss of biological utility or activity and possibly with an increase in such utility or activity. In some embodiments, the amino acid sequences substantially homologous to a particular amino acid sequence are at least 70%, e.g., 75%, 80%85%, 90%, 95% or another percent from 70% to 100%, in integers thereof, identical to the particular amino acid sequence.

As used herein, “Lhx3” is refers to the Lhx3 protein of Genebank accession No: NP_055379.1; (human) NP_001034742.1 (mouse) encoded by genes NM_014564 (human) NM_001039653.1 (mouse). The term Lhx3 also encompasses species variants, homologues, allelic forms, mutant forms, and equivalents thereof, including conservative substitutions, additions, deletions therein not adversely affecting the structure of function. Lhx3 is referred in the art as aliases; Homo sapiens LIM homeobox 3 (LHX3), transcript variant 2, mRNA, CPHD3; LIM3; M2-LHX3. In addition to naturally-occurring allelic variants of the Lhx3 sequences that may exist in the population, it will be appreciated that, as is the case for virtually all proteins, a variety of changes can be introduced into the human or mouse Lhx3 sequences (referred to as “wild type” sequences) without substantially altering the functional (biological) activity of the polypeptides. Such variants are included within the scope of the terms “Lhx3”, “Lhx3 protein”, etc.

As used herein, “Ascl1” is refers to the Ascl1 protein of Genebank accession No: NP_004307.2 (human), or NP_032579.2 (mouse) and is encoded by genes NM_004316.3 (human) or NM_008553.4 (mouse), respectively. The term Ascl1 also encompasses species variants, homologues, allelic forms, mutant forms, and equivalents thereof, including conservative substitutions, additions, deletions therein not adversely affecting the structure of function. Ascl1 is referred in the art as aliases; Homo sapiens achaete-scute complex homolog 1 (Drosophila) (ASCL1), ASH1; bHLHa46; HASH1; MASH1. In addition to naturally-occurring allelic variants of the Ascl1 sequences that may exist in the population, it will be appreciated that, as is the case for virtually all proteins, a variety of changes can be introduced into the human or mouse Ascl1 sequences (referred to as “wild type” sequences) without substantially altering the functional (biological) activity of the polypeptides. Such variants are included within the scope of the terms “Ascl1”, “Ascl1 protein”, etc.

As used herein, “Brn2” is refers to the Brn2 protein of Genebank accession No: NP_005595.2 (human) or NP_032925.1 (mouse) and encoded by genes NM_005604.2 (human) or NM_008899.1 (mouse), respectively. The term Brn2 also encompasses species variants, homologues, allelic forms, mutant forms, and equivalents thereof, including conservative substitutions, additions, deletions therein not adversely affecting the structure of function. Brn2 is referred in the art as aliases; POU3F2, POU class 3 homeobox 2, BRN2, OCT7, POUF3. In addition to naturally-occurring allelic variants of the Brn2 sequences that may exist in the population, it will be appreciated that, as is the case for virtually all proteins, a variety of changes can be introduced into the human or mouse Brn2 sequences (referred to as “wild type” sequences) without substantially altering the functional (biological) activity of the polypeptides. Such variants are included within the scope of the terms “Brn2”, “Brn2 protein”, etc.

As used herein, “Myt1l” refers to the Myt1l protein of Genebank accession No: NP_055840.2 (human) or NP_001087244.1 (mouse) and encoded by genes NM_015025.2 (human) or NM_001093775.1 (mouse), respectively. The term Myt1l also encompasses species variants, homologues, allelic forms, mutant forms, and equivalents thereof, including conservative substitutions, additions, deletions therein not adversely affecting the structure of function. Myt1l is referred in the art as aliases; myelin transcription factor 1-like (MYT1L), KIAA1106, “neural zinc finger transcription factor 1”, NZF1. In addition to naturally-occurring allelic variants of the Myt1l sequences that may exist in the population, it will be appreciated that, as is the case for virtually all proteins, a variety of changes can be introduced into the human or mouse Myt1l sequences (referred to as “wild type” sequences) without substantially altering the functional (biological) activity of the polypeptides. Such variants are included within the scope of the terms “Myt1l”, “Myt1l protein”, etc.

As used herein, “Isl1” is refers to the Isl1 protein of Genebank accession No: NP_002193.2 (human) or NP_067434.3 (mouse) and is encoded by genes NM_002202.2 (human) or NM_021459.4 (mouse) respectively. The term Isl1 also encompasses species variants, homologues, allelic forms, mutant forms, and equivalents thereof, including conservative substitutions, additions, deletions therein not adversely affecting the structure of function. Isl1 is referred in the art as aliases; ISL LIM homeobox 1, Isl-1, ISLET 1. In addition to naturally-occurring allelic variants of the Isl1 sequences that may exist in the population, it will be appreciated that, as is the case for virtually all proteins, a variety of changes can be introduced into the human or mouse Isl1 sequences (referred to as “wild type” sequences) without substantially altering the functional (biological) activity of the polypeptides. Such variants are included within the scope of the terms “Isl1”, “Isl1 protein”, etc.

As used herein, “Hb9” is refers to the Hb9 protein of Genebank accession No: NP_001158727.1 (human) or NP_064328.2 (mouse) and encoded by genes NM_001165255.1 (human) or NM_019944.2 (mouse) respectively. The term Hb9 also encompasses species variants, homologues, allelic forms, mutant forms, and equivalents thereof, including conservative substitutions, additions, deletions therein not adversely affecting the structure of function. Hb9 is referred in the art as aliases; motor neuron and pancreas homeobox 1, MNX1, HB9, HOXHB9, SCRA1. In addition to naturally-occurring allelic variants of the H 9 sequences that may exist in the population, it will be appreciated that, as is the case for virtually all proteins, a variety of changes can be introduced into the human or mouse Hb9 sequences (referred to as “wild type” sequences) without substantially altering the functional (biological) activity of the polypeptides. Such variants are included within the scope of the terms “Hb9”, “Hb9 protein”, etc.

As used herein, “Ngn2” is refers to the Ngn2 protein of Genebank accession No: NP_076924.1 (human) or NP_033848.1 (mouse) and are encoded by NM_024019.2 (human) or NM_009718.2 (mouse), respectively. The term Ngn2 also encompasses species variants, homologues, allelic forms, mutant forms, and equivalents thereof, including conservative substitutions, additions, deletions therein not adversely affecting the structure of function. Ngn2 is referred in the art as aliases; Neurogenin 2 (NEUROG2), Atoh4, bHLHa8, Math4A, ngn-2. In addition to naturally-occurring allelic variants of the Ngn2 sequences that may exist in the population, it will be appreciated that, as is the case for virtually all proteins, a variety of changes can be introduced into the human or mouse Ngn2 sequences (referred to as “wild type” sequences) without substantially altering the functional (biological) activity of the polypeptides. Such variants are included within the scope of the terms “Ngn2”, “Ngn2 protein”, etc.

As used herein, “NeuroD1” is refers to the NewroDiprotein of Genebank accession No: NP_002491.2 (human) or NP_035024.1 (mouse) and encoded by genes NM_002500.3 (human) or NM_010894.2 (mouse), respectively. The term NeuroD1 also encompasses species variants, homologues, allelic forms, mutant forms, and equivalents thereof, including conservative substitutions, additions, deletions therein not adversely affecting the structure of function. NeuroD1 is referred in the art as aliases; neurogenic differentiation 1, beta-cell E-box transactivator 2”, BETA2, BHF-1, bHLHa3, MODY6, NeuroD, “neurogenic helix-loop-helix protein NEUROD”. In addition to naturally-occurring allelic variants of the NeuroD1 sequences that may exist in the population, it will be appreciated that, as is the case for virtually all proteins, a variety of changes can be introduced into the human or mouse NeuroD1 sequences (referred to as “wild type” sequences) without substantially altering the functional (biological) activity of the polypeptides. Such variants are included within the scope of the terms “NeuroD1”, “NeuroD1 protein”, etc.

The term a “variant” in referring to a polypeptide could be, e.g., a polypeptide at least 80%, 85%, 90%, 95%, 98%, or 99% identical to full length polypeptide. The variant could be a fragment of full length polypeptide, e.g., a fragment of at least 10 or at least 20 contagious amino acids of the wild type version of the polypeptide. In some embodiments, a variant is a naturally occurring splice variant. The variant could be a polypeptide at least 80%, 85%, 90%, 95%, 98%, or 99% identical to a fragment of the polypeptide, wherein the fragment is at least 50%, 60%, 70%, 80%, 85%, 90%, 95%, 98%, or 99% as long as the full length wild type polypeptide or a domain thereof having an activity of interest such as the ability to directly convert fibroblasts to iMNs. In some embodiments the domain is at least 100, 200, 300, or 400 amino acids in length, beginning at any amino acid position in the sequence and extending toward the C-terminus. Variations known in the art to eliminate or substantially reduce the activity of the protein are preferably avoided. In some embodiments, the variant lacks an N- and/or C-terminal portion of the full length polypeptide, e.g., up to 10, 20, or 50 amino acids from either terminus is lacking. In some embodiments the polypeptide has the sequence of a mature (full length) polypeptide, which means a polypeptide that has had one or more portions such as a signal peptide removed during normal intracellular proteolytic processing (e.g., during co-translational or post-translational processing). In some embodiments wherein the protein is produced other than by purifying it from cells that naturally express it, the protein is a chimeric polypeptide, which means that it contains portions from two or more different species. In some embodiments wherein a protein is produced other than by purifying it from cells that naturally express it, the protein is a derivative, which means that the protein comprises additional sequences not related to the protein so long as those sequences do not substantially reduce the biological activity of the protein.

One of skill in the art will be aware of, or will readily be able to ascertain, whether a particular polypeptide variant, fragment, or derivative is functional using assays known in the art. For example, the ability of a variant of a Lhx3, Ascl1, Brn2, Myt1l, Isl1, Hb9, Ngn2 or NeuroD1 polypeptides to convert a somatic cell, e.g., fibroblast to a iMN can be assessed using the assays as disclose herein in the Examples. Other convenient assays include measuring the ability to activate transcription of a reporter construct containing a Lhx3, Ascl1, Brn2, Myt1l, Isl1, Hb9, Ngn2 or NeuroD1 binding site operably linked to a nucleic acid sequence encoding a detectable marker such as luciferase. One assay involves determining whether the Lhx3, Ascl1, Brn2, Myt1l, Isl1, Hb9, Ngn2 or NeuroD variant induces a somatic cell, e.g., fibroblast to become a iMN or express markers of a motor neuron or exhibit functional characteristics of a motor neuron as disclosed herein. Determination of such expression of MN markers can be determined using any suitable method, e.g., immunoblotting. Such assays may readily be adapted to identify or confirm activity of agents that directly convert a somatic cell, e.g., fibroblast to a iMN. In certain embodiments of the invention a functional variant or fragment has at least 50%, 60%, 70%, 80%, 90%, 95% or more of the activity of the full length wild type polypeptide.

The term “functional fragments” as used herein regarding Lhx3, Ascl1, Brn2, Myt1l, Isl1, Hb9, Ngn2 or NeuroD1 polypeptides having amino acid sequences substantially homologous thereto means a polypeptide sequence of at least 5 contiguous amino acids of the Lhx3, Ascl1, Brn2, Myt1l, Isl1, Hb9, Ngn2 or NeuroD1 having amino acid sequences substantially homologous thereto, wherein the functional fragment polypeptide sequence is about at least 50%, or 60% or 70% or at 80% or 90% or 100% or greater, for example 1.5-fold, 2-fold, 3-fold, 4-fold or greater than 4-fold as effective at direct conversion of a somatic cell, e.g., fibroblast to a iMN as the corresponding wild type Lhx3, Ascl1, Brn2, Myt1l, Isl1, Hb9, Ngn2 or NeuroD1 polypeptides, as described herein. The functional fragment polypeptide may have additional functions that can include decreased antigenicity, increased DNA binding (as in transcription factors), or altered RNA binding (as in regulating RNA stability or degradation).

The term “vector” refers to a carrier DNA molecule into which a DNA sequence can be inserted for introduction into a host cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g. EPV and EBV vectors.

As used herein, the term “adenovirus” refers to a virus of the family Adenovirida. Adenoviruses are medium-sized (90-100 nm), nonenveloped (naked) icosahedral viruses composed of a nucleocapsid and a double-stranded linear DNA genome.

As used herein, the term “non-integrating viral vector” refers to a viral vector that does not integrate into the host genome; the expression of the gene delivered by the viral vector is temporary. Since there is little to no integration into the host genome, non-integrating viral vectors have the advantage of not producing DNA mutations by inserting at a random point in the genome. For example, a non-integrating viral vector remains extra-chromosomal and does not insert its genes into the host genome, potentially disrupting the expression of endogenous genes. Non-integrating viral vectors can include, but are not limited to, the following: adenovirus, alphavirus, picornavirus, and vaccinia virus. These viral vectors are “non-integrating” viral vectors as the term is used herein, despite the possibility that any of them may, in some rare circumstances, integrate viral nucleic acid into a host cell's genome. What is critical is that the viral vectors used in the methods described herein do not, as a rule or as a primary part of their life cycle under the conditions employed, integrate their nucleic acid into a host cell's genome. It goes without saying that an iPS cell generated by a non-integrating viral vector will not be administered to a subject unless it and its progeny are free from viral remnants.

As used herein, the term “viral remnants” refers to any viral protein or nucleic acid sequence introduced using a viral vector. Generally, integrating viral vectors will incorporate their sequence into the genome; such sequences are referred to herein as a “viral integration remnant”. However, the temporary nature of a non-integrating virus means that the expression, and presence of, the virus is temporary and is not passed to daughter cells. Thus, upon passaging of a re-programmed cell the viral remnants of the non-integrating virus are essentially removed.

As used herein, the term “free of viral integration remnants” and “substantially free of viral integration remnants” refers to iPS cells that do not have detectable levels of an integrated adenoviral genome or an adenoviral specific protein product (i.e., a product other than the gene of interest), as assayed by PCR or immunoassay. Thus, the iPS cells that are free (or substantially free) of viral remnants have been cultured for a sufficient period of time that transient expression of the adenoviral vector leaves the cells substantially free of viral remnants.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

As used herein, the term “tissue-specific promoter” means a nucleic acid sequence that serves as a promoter, i.e., regulates expression of a selected nucleic acid sequence operably linked to the promoter, and which selectively affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause lesser expression in other tissues as well.

The term “phenotype” refers to one or a number of total biological characteristics that define the cell or organism under a particular set of environmental conditions and factors, regardless of the actual genotype.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, “the presence of lower amounts of a marker in the iMN as compared to the somatic cell, e.g., fibroblast from which the iMN was derived” refers to an amount of a marker protein or gene product (e.g. mRNA) that is significantly decreased in the iMN as compared to the amount of the same marker present in the somatic cell, e.g., fibroblast from which is was derived. The term “significantly decreased” means that the differences between the compared levels is statistically significant. The levels of the marker level can be represented by arbitrary units, for example as units obtained from a densitometer, luminometer, or an Elisa plate reader. As a non-limiting example, a iMN has significantly decreased levels of Snail1, thy1, Fsp1 expression as compared to a fibroblast from which it was derived.

As used herein, “the presence of higher amounts of a marker in the iMN as compared to the somatic cell, e.g., fibroblast from which is was derived” refers to an amount of a marker protein or gene product (e.g. mRNA) that is significantly increased in the iMN as compared to the amount of the same marker present in the somatic cell, e.g., fibroblast from which is was derived. The phrase “significantly increased” means that the differences between the compared levels is statistically significant. The levels of the marker level can be represented by arbitrary units, for example as units obtained from a densitometer, luminometer, or an Elisa plate reader. As a non-limiting example, a iMN has significantly increased levels of P2-tubilins (e.g, Tubb2a and Tubb2b), Map2, synapsins (e.g., Syn1 and Syn2), synaptophysin, synaptotagmins (e.g., Syt1, Syt4, Syt13, Syt 16), NeuroD, Isl1, cholineacetyltransferase (ChAT), e.g., vascular ChAT (VChAT) as compared to a fibroblast from which it was derived.

As used herein, the term “transcription factor” refers to a protein that binds to specific parts of DNA using DNA binding domains and is part of the system that controls the transfer (or transcription) of genetic information from DNA to RNA.

As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “lineages” as used herein describes a cell with a common ancestry or cells with a common developmental fate. In the context of a cell that is of “neuronal linage” this means the cell can differentiate along the neuronal lineage restricted pathways.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased” “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “xenogeneic” refers to cells that are derived from different species.

The term “iMN inducing factor” refers to a gene, RNA, or protein that promotes or contributes to direct conversion or transdifferentiation of a somatic cell to a iMN. In aspects of the invention relating to reprogramming factor(s), the invention provides embodiments in which the iMN-inducing factors of interest for transdifferentiation of somatic cells to iMN in vitro.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions”. To ensure an effective selection, a population of cells can be maintained for a under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

A “reporter gene” as used herein encompasses any gene that is genetically introduced into a cell that adds to the phenotype of the stem cell. Reporter genes as disclosed in this invention are intended to encompass fluorescent, luminescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny. A reporter gene is generally operatively linked to sequences that regulate its expression in a manner dependent upon one or more conditions which are monitored by measuring expression of the reporter gene. In some cases, expression of the reporter gene may be determined in live cells. Where live cell reporter gene assays are used, reporter gene expression may be monitored at multiple timepoints, e.g., 2, 3, 4, 5, 6, 8, or 10 or more timepoints. In some cases, where a live cell reporter assay is used, reporter gene expression is monitored with a frequency of at least about 10 minutes to about 24 hours, e.g., 20 minutes, 1 hour, 2 hours, 3 hours, 4 hours, 5 hours, 6 hours, 7 hours, 8 hours, 9 hours, 10 hours, 12 hours, 18 hours, or another frequency from any integer between about 10 minutes to about 24 hours.

The terms “subject” and “individual” are used interchangeably herein, and refer to an animal, for example, a human from whom cells can be obtained and/or to whom treatment, including prophylactic treatment, with the cells as described herein, is provided. For treatment of those conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the term “treating” refer to providing medical or surgical attention, care, or management to an individual. The individual is usually ill or injured, or at increased risk of becoming ill relative to an average member of the population and in need of such attention, care, or management.

In some embodiments, the term “treating” and “treatment” refers to administering to a subject an effective amount of a composition, e.g., a composition comprising iN or iMN or their differentiated progeny so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, treatment can be “prophylaxic treatment, where the subject is administered a composition as disclosed herein (e.g., a population of iN or iMN or their progeny) to a subject at risk of developing a neuron disease (e.g., a motor neuron disease) as disclosed herein. In some embodiments, treatment is “effective” if the progression of a disease is reduced or halted. Those in need of treatment include those already diagnosed with a motor neuron disease or disorder, e.g., ALS or SMA, as well as those likely to develop a motor neuron disease or disorder due to genetic susceptibility or other factors such as family history of motor neuron disease, exposure to susceptibility factors, weight, diet and health.

As used herein, the terms “administering,” “introducing” and “transplanting” are used interchangeably in the context of the placement of iNs or iMNs of the invention into a subject, by a method or route which results in at least partial localization of the iN or iMN at a desired site. In some embodiments, the iN or iMNs can be placed directly in the spinal cord or in the cerebellum, or alternatively be administered by any appropriate route which results in delivery to a desired location in the subject where at least a portion of the cells or components of the cells remain viable. The period of viability of the cells after administration to a subject can be as short as a few hours, e. g. twenty-four hours, to a few days, to as long as several or more years.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of iMNs and/or their progeny and/or compound and/or other material other than directly into the central nervous system, such that it enters the animal's system and, thus, is subject to metabolism and other like processes, for example, subcutaneous administration.

The term “tissue” refers to a group or layer of specialized cells which together perform certain special functions. The term “tissue-specific” refers to a source of cells from a specific tissue.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the disclosure. Further, all patents, patent applications, and publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

Direct Reprogramming (Transdifferentiation)

The process of altering the cell phenotype of a differentiated cell (i.e. a first cell), e.g., altering the phenotype of a somatic cell to a differentiated cell of a different phenotype (i.e. a second cell) without the first differentiated cell being completely reprogrammed to a less differentiated phenotype intermediate is referred to as “direct reprogramming” or “transdifferentiation”. Stated another way, cells of one type can be converted to another type in a process by what is commonly referred to in the art as transdifferentiation, cellular reprogramming or lineage reprogramming.

Transdifferentiation encompasses a process of switching the phenotype of a first differentiated cell to the phenotype of a second different differentiated cell, without the complete reversal of the differentiation state of the somatic cell, and is different from “reprogramming a cell to a pluripotent state” which typically refers to a process which partially or completely reverses the differentiation state of a somatic cell to a cell with a stem cell-like phenotype, e.g., to an induced pluripotent stem cell (iPSC).

The disclosure relates to compositions and methods for the direct conversion of a non-neuronal cell (e.g., from a less differentiated cell such as a stem cell or pluripotent cell or from an alternate cell type such as a non-neuronal somatic cell) to a functional neuron, referred to herein as an “induced neuron (iN)”. In certain embodiments of the invention, the conversion (e.g., transdifferentiation) of a non-neuronal cell, e.g., somatic cell, e.g., fibroblast causes the non-neuronal cell, e.g., somatic cell, e.g., fibroblast to assume a iN like state, without being completely reprogrammed to a pluripotent state prior to becoming an iN.

In some embodiments, the methods and compositions of the disclosure can be practiced on non-neuronal cells that are fully differentiated and/or restricted to giving rise only to cells of that particular type. The non-neuronal cells can be either partially or terminally differentiated prior to direct conversion to iNs. In some embodiments, non-neuronal cells which are transdifferentiated into iNs are somatic cells (e.g., fibroblast cells).

In some embodiments, the methods and compositions of the disclosure can be practiced on somatic cells that are fully differentiated and/or restricted to giving rise only to cells of that particular type. The somatic cells can be either partially or terminally differentiated prior to direct conversion to iNs. In some embodiments, somatic cells which are transdifferentiated into iNs are fibroblast cells.

The disclosure relates to compositions and methods for direct conversion of a non-neuronal cell (e.g., somatic cell) to a functional neuron. In some embodiments, the disclosure provides methods for direct conversion of fibroblasts to a different phenotype, such as an iN.

The disclosure also relates to compositions and methods for the direct conversion of a somatic cell, e.g., a fibroblast to a functional motor neuron, referred to herein as an “induced motor neuron (iMN)”. In certain embodiments of the invention, the transdifferentiation of a somatic cell, e.g., fibroblast causes the somatic cell to assume a MN like state, without being completely reprogrammed to a pluripotent state prior to becoming an iMN.

In some embodiments, the methods and compositions of the disclosure can be practiced on somatic cells that are fully differentiated and/or restricted to giving rise only to cells of that particular type. The somatic cells can be either partially or terminally differentiated prior to direct conversion to iMNs. In some embodiments, somatic cells which are transdifferentiated into iMNs are fibroblast cells.

The disclosure relates to compositions and methods for direct conversion of a somatic cell, e.g., a fibroblast to a functional motor neuron. In some embodiments, the disclosure provides methods for direct conversion of fibroblasts to a different phenotype, such as an iMN.

Direct Conversion of Fibroblasts to iMNs or iNs

The disclosure relates to a method of converting (e.g., transdifferentiating) non-neuronal cells (e.g., fibroblast cells, e.g., fibroblasts) to neurons, referred to herein as iNs (induced neurons). In some embodiments, a non-neuronal cell, e.g., somatic cell are the preferred starting material. In some embodiments, a population of iNs are produced by inhibiting the level or activity of ALK4, ALK5, and ALK7 in a non-neuronal cell, e.g., somatic cell. In some embodiments, a population of iNs are produced by inhibiting the level or activity of PLK1 in a non-neuronal cell, e.g., fibroblast. In some embodiments, a population of iNs are produced by inhibiting the level or activity of ALK4, ALK5, ALK7, and PLK1 in a non-neuronal cell. In alternative embodiments, the population of a non-neuronal cell can comprise a mixture or combination of different non-neuronal cells (for example a mixture of cells such as a fibroblasts and other somatic cells).

The disclosure relates to a method of converting somatic cells, e.g., fibroblasts to motor neurons, referred to herein as iMNs (induced motor neurons). In some embodiments, a somatic cell, e.g., fibroblast are the preferred starting material. In some embodiments, a population of iMNs are produced by inhibiting the level or activity of ALK4, ALK5, and ALK7 in a somatic cell, e.g., fibroblast. In some embodiments, a population of iMNs are produced by inhibiting the level or activity of PLK1 in a somatic cell, e.g., fibroblast. In some embodiments, a population of iMNs are produced by inhibiting the level or activity of ALK4, ALK5, ALK7 and PLK1 in a somatic cell, e.g., fibroblast. In alternative embodiments, the population of a somatic cell, e.g., fibroblast can comprise a mixture or combination of different a somatic cells, e.g., fibroblast, for example a mixture of cells such as a fibroblasts and other somatic cells.

In some embodiments, the population of a non-neuronal cells is a substantially pure population of non-neuronal cells. In some embodiments, a population of a non-neuronal cells is a population of non-neuronal cells or differentiated cells. In some embodiments, the population of non-neuronal cells, e.g., somatic cells are substantially free or devoid of embryonic stem cells or pluripotent cells or iPS cells.

In some embodiments, the population of a somatic cell, e.g., fibroblast is a substantially pure population of fibroblasts. In some embodiments, a population of a somatic cell, e.g., fibroblast is a population of somatic cells or differentiated cells. In some embodiments, the population of a somatic cell, e.g., fibroblast are substantially free or devoid of embryonic stem cells or pluripotent cells or iPS cells.

In some embodiments, a non-neuronal cell is genetically modified. In some embodiments, the non-neuronal cell comprises one or more nucleic acid sequences encoding at the proteins of least three MN-inducing factors selected from Lhx3, Ascl1, Brn2, Myt1l, Isl1, Hb9, Ngn2 or NeuroD1 or functional variants or functional fragments thereof, as shown in Table 1.

In some embodiments, a somatic cell, e.g., fibroblast is genetically modified. In some embodiments, the somatic cell, e.g., fibroblast comprises one or more nucleic acid sequences encoding at the proteins of least three MN-inducing factors selected from Lhx3, Ascl1, Brn2, Myt1l, Isl1, Hb9, Ngn2 or NeuroD1 or functional variants or functional fragments thereof, as shown in Table 1.

TABLE 1
MN-inducing factors

| MN- | | | | Human | Mouse |
|---|---|---|---|---|---|
| inducing | | | | nucleic | nucleic |
| factor | Gene synonyms | Human protein | Mouse protein | acid | acid |
| Lhx3 | Homo sapiens | NP_055379.1 | NP_001034742.1 | NM_014564 | NM_001039653.1 |
| | LIM homeobox3 | | | | |
| | (LHX3), | | | | |
| | transcript | | | | |
| | variant 2, mRNA, | | | | |
| | CPHD3; LIM3; | | | | |
| | M2-LHX3 | | | | |
| Ascll | Homo sapiens | NP_004307.2 | NP_032579.2 | NM_004316.3 | NM_008553.4 |
| | achaete-scute | | | | |
| | complex | | | | |
| | homolog 1 | | | | |
| | (Drosophila) | | | | |
| | (ASCL1), ASH; | | | | |
| | bHLHa46; | | | | |
| | HASH1; | | | | |
| | MASH1 | | | | |
| Brn2 | POU3F2, POU | NP_005595.2 | NP_032925.1 | NM_005604.2 | NM_008899.1 |
| | class 3 | | | | |
| | homeobox 2, | | | | |
| | BRN2, OCT7, | | | | |
| | POUF3 | | | | |
| Mytl1 | myelin | NP_055840.2 | NP_001087244.1 | NM_015025.2 | NM_001093775.1 |
| | transcription | | | | |
| | factor 1-like | | | | |
| | (MYT1L), | | | | |
| | KIAA1106, | | | | |
| | “neural zinc | | | | |
| | finger | | | | |
| | transcription | | | | |
| | factor 1”, NZF1 | | | | |
| Isl1 | ISL LIM | NP_002193.2 | NP_067434.3 | NM_002202.2 | NM_021459.4 |
| | homeobox 1, Isl- | | | | |
| | 1, ISLET1 | | | | |
| Hb9 | motor neuron and | NP_001158727.1 | NP_064328.2 | NM_001165255.1 | NM_019944.2 |
| | pancreas | | | | |
| | homeobox 1, | | | | |
| | MNX1, HB9, | | | | |
| | HOXHB9, | | | | |
| | SCRA1 | | | | |
| Ngn2 | Neurogenin 2 | NP_076924.1 | NP_033848.1 | NM_024019.2 | NM_009718.2 |
| | (NEUROG2), | | | | |
| | Atoh4, bHLHa8, | | | | |
| | Math4A, ngn-2. | | | | |
| NeuroDI | neurogenic | NP 002491.2 | NP_035024.1 | NM_002500.3 | NM_010894.2 |
| | differentiation 1, | | | | |
| | beta-cell E-box | | | | |
| | transactivator 2”, | | | | |
| | BETA2, BHF-1, | | | | |
| | bHLHa3, | | | | |
| | MODY6, | | | | |
| | NeuroD, | | | | |
| | “neurogenic | | | | |
| | helix-loop-helix | | | | |
| | protein | | | | |
| | NEUROD”. | | | | |

In some embodiments, a non-neuronal cell (e.g., somatic cell, e.g., fibroblast) can be isolated from a subject, for example as a tissue biopsy, such as, for example, a skin biopsy. In some embodiments, the a non-neuronal cells are maintained in culture by methods known by one of ordinary skill in the art, and in some embodiments, propagated prior to being directly converted into iNs or iMNs by the methods as disclosed herein.

Further, a non-neuronal cell, e.g., fibroblast can be from any mammalian species, with non-limiting examples including a murine, bovine, simian, porcine, equine, ovine, or human cell. For clarity and simplicity, the description of the methods herein refers to a mammalian non-neuronal cell (e.g., somatic cell, e.g., fibroblast) but it should be understood that all of the methods described herein can be readily applied to other cell types of non-neuronal cells. In one embodiment, the non-neuronal cell, e.g., somatic cell is derived from a human individual. In one embodiment, the non-neuronal cell, e.g., somatic cell is derived from a human individual, wherein the suitable MN-inducing factors are human (e.g., human Lhx3, Ascl1, Brn2, Myt1l, Isl1, Hb9, Ngn2 or NeuroD1 polypeptides respectively). In alternative embodiments, the non-neuronal cell, e.g., somatic cell is derived from a mouse subject, and wherein the suitable MN-inducing factors are mouse (e.g., mouse Lhx3, Ascl1, Brn2, Myt1l, Isl1, Hb9, Ngn2 or NeuroD1 polypeptides respectively). In some embodiments, mouse MN-inducing factors can be used to directly convert human non-neuronal cell, e.g., somatic cell to iMNs and vice versa, human MN-inducing factors can be used for conversion of mouse fibroblasts into iMNs. In some embodiments, any combination of mouse or human MN-inducing factors can be used for conversion of mouse or human non-neuronal cells, e.g., somatic cells into iMNs.

In some embodiments, at least one MN-inducing factor is used in the method for conversion (e.g., transdifferentiation) of a non-neuronal cell, e.g., somatic cell to a iN (e.g., iMN) according to the methods as disclosed herein. In some embodiments, at least 2, or at least 3, or at least 4, or at least 5, or at least 6, or at least 7, or at least 8 MN-inducing factors selected from any of the group consisting of Lhx3, Ascl1, Brn2, Myt1l, Isl1, Hb9, Ngn2 or NeuroD1 are used in the methods of conversion of a non-neuronal cell, e.g., somatic cell to a iN according to the methods as disclosed herein.

In some embodiments, at least one MN-inducing factor is used in the method for transdifferentiation of a somatic cell, e.g., a fibroblast to a iMN according to the methods as disclosed herein. In some embodiments, at least 2, or at least 3, or at least 4, or at least 5, or at least 6, or at least 7, or at least 8 MN-inducing factors selected from any of the group consisting of Lhx3, Ascl1, Brn2, Myt1l, Isl1, Hb9, Ngn2 or NeuroD1 are used in the methods of transdifferentiation of a somatic cell, e.g., a fibroblast to a iMN according to the methods as disclosed herein.

In some embodiments, Lhx3 and Ascl1 are used with any combination of other MN-inducing factor selected from the group of Brn2, Myt1l, Isl1, Hb9, Ngn2 or NeuroD1. In some embodiments, Ascl1, Lhx3 MN-inducing agents are used with Brn2, and Myt1l in the methods to convert a non-neuronal cell, e.g., somatic cell to a iN. In some embodiments to increase efficiency of conversion (e.g., transdifferentiation), any one or more of a combination of the MN-inducing factors selected from Isl1, Hb9 and Ngn2 can also be used with Ascl1, Lhx3, Brn2, and Myt1l MN-inducing factors. In some embodiments, Myt1l and/or Brn2 and/or Isl1 are not used as MN-inducing factors in the methods as disclosed herein. Additionally, in some embodiments, miR-124 is not used as a MN-inducing agent. In some embodiments, for conversion of human somatic cells, e.g., human fibroblasts, NeuroD1 is used as one of the MN-inducing agents.

In some embodiments, Lhx3 and Ascl1 are used with any combination of other MN-inducing factor selected from the group of Brn2, Myt1l, Isl1, Hb9, Ngn2 or NeuroD1. In some embodiments, Ascl1, Lhx3 MN-inducing agents are used with Brn2, and Myt1l in the methods to transdifferentiate a somatic cell, e.g., a fibroblast to a iMN. In some embodiments to increase efficiency of transdifferentiation, any one or more of a combination of the MN-inducing factors selected from Isl1, Hb9 and Ngn2 can also be used with Ascl1, Lhx3, Brn2, and Myt1l MN-inducing factors. In some embodiments, Myt1l and/or Brn2 and/or Isl1 are not used as MN-inducing factors in the methods as disclosed herein. Additionally, in some embodiments, miR-124 is not used as a MN-inducing agent. In some embodiments, for transdifferentiation of human somatic cells, e.g., human fibroblasts, NeuroD1 is used as one of the MN-inducing agents.

In some embodiments, a ALK4, ALK5, and ALK7 inhibitor is used with any combination of other MN-inducing factors selected from the group of Lhx3, Ascl1, Brn2, Myt1l, Isl1, Hb9, Ngn2 or NeuroD1 in the methods to convert a non-neuronal cell, e.g., somatic cell to a iN to increase efficiency of neuron formation or production. In some embodiments, a ALK4, ALK5, and ALK7 inhibitor is used with any combination of other MN-inducing factors selected from the group of Lhx3, Ascl1, Brn2, Myt1l, Isl1, Hb9, Ngn2 or NeuroD1 in the methods to convert a non-neuronal cell, e.g., somatic cell to a iMN to increase the rate (or efficiency) of induced motor neuron formation. In some embodiments, efficiency of transdifferentiation is increased by at least 2.5 fold. In some embodiments, efficiency of transdifferentiation is increased by at least 3.0 fold. In some embodiments, efficiency of transdifferentiation is increased by at least 3.5 fold. In some embodiments, efficiency of transdifferentiation is increased by at least 4.0 fold. In some embodiments, efficiency of transdifferentiation is increased by at least 4.5 fold. In some embodiments, efficiency of transdifferentiation is increased by at least 5.0 fold. In some embodiments, efficiency of transdifferentiation is increased by at least 5.5 fold. In some embodiments, efficiency of transdifferentiation is increased by at least 6.0 fold. In some embodiments, efficiency of transdifferentiation is increased by at least 6.5 fold. In some embodiments, efficiency of transdifferentiation is increased by at least 7.0 fold. In some embodiments, efficiency of transdifferentiation is increased by at least 7.5 fold. In some embodiments, efficiency of transdifferentiation is increased by at least 8.0 fold. In some embodiments, efficiency of transdifferentiation is increased by at least 8.5 fold. In some embodiments, efficiency of transdifferentiation is increased by at least 9.0 fold. In some embodiments, efficiency of transdifferentiation is increased by at least 9.5 fold. In some embodiments, efficiency of transdifferentiation is increased by at least 10.0 fold.

In some embodiments, a PLK1 inhibitor is used with any combination of other MN-inducing factors selected from the group of Lhx3, Ascl1, Brn2, Myt1l, Isl1, Hb9, Ngn2 or NeuroD1 in the methods to convert a non-neuronal cell, e.g., somatic cell to a iN to increase efficiency of conversion. In some embodiments, a PLK1 inhibitor is used with any combination of other MN-inducing factors selected from the group of Lhx3, Ascl1, Brn2, Myt1l, Isl1, Hb9, Ngn2 or NeuroD1 in the methods to convert a non-neuronal cell, e.g., somatic cell to a iMN to increase rate (or efficiency) of conversion. In some embodiments, efficiency of transdifferentiation is increased by at least 2.5 fold. In some embodiments, efficiency of transdifferentiation is increased by at least 3.0 fold. In some embodiments, efficiency of transdifferentiation is increased by at least 3.5 fold. In some embodiments, efficiency of transdifferentiation is increased by at least 4.0 fold. In some embodiments, efficiency of transdifferentiation is increased by at least 4.5 fold. In some embodiments, efficiency of transdifferentiation is increased by at least 5.0 fold. In some embodiments, efficiency of transdifferentiation is increased by at least 5.5 fold. In some embodiments, efficiency of transdifferentiation is increased by at least 6.0 fold. In some embodiments, efficiency of transdifferentiation is increased by at least 6.5 fold. In some embodiments, efficiency of transdifferentiation is increased by at least 7.0 fold. In some embodiments, efficiency of transdifferentiation is increased by at least 7.5 fold. In some embodiments, efficiency of transdifferentiation is increased by at least 8.0 fold. In some embodiments, efficiency of transdifferentiation is increased by at least 8.5 fold. In some embodiments, efficiency of transdifferentiation is increased by at least 9.0 fold, in some embodiments, efficiency of transdifferentiation is increased by at least 9.5 fold. In some embodiments, efficiency of transdifferentiation is increased by at least 10.0 fold.

In some embodiments, a ALK4, ALK5, and ALK7 inhibitor and a PLK1 inhibitor is used with any combination of other MN-inducing factors selected from the group of Lhx3, Ascl1, Brn2, Myt1l, Isl1, Hb9, Ngn2 or NeuroD1 in the methods to convert a non-neuronal cell, e.g., somatic cell to a iN to increase the rate (or efficiency) of conversion. In some embodiments, a ALK4, ALK5, and ALK7 inhibitor and a PLK1 inhibitor is used with any combination of other MN-inducing factors selected from the group of Lhx3, Ascl1, Brn2, Myt1l, Isl1, Hb9, Ngn2 or NeuroD1 in the methods to convert a non-neuronal cell, e.g., somatic cell to a iMN to increase the rate (or efficiency) of induced motor neuron formation. In some embodiments, efficiency of transdifferentiation is increased by at least 25 fold. In some embodiments, efficiency of transdifferentiation is increased by at least 30 fold. In some embodiments, efficiency of transdifferentiation is increased by at least 35 fold. In some embodiments, efficiency of transdifferentiation is increased by at least 40 fold. In some embodiments, efficiency of transdifferentiation is increased by at least 41 fold. In some embodiments, efficiency of transdifferentiation is increased by at least 42 fold. In some embodiments, efficiency of transdifferentiation is increased by at least 43 fold. In some embodiments, efficiency of transdifferentiation is increased by at least 44 fold. In some embodiments, efficiency of transdifferentiation is increased by at least 45 fold. In some embodiments, efficiency of transdifferentiation is increased by at least 46 fold. In some embodiments, efficiency of transdifferentiation is increased by at least 47 fold. In some embodiments, efficiency of transdifferentiation is increased by at least 48 fold. In some embodiments, efficiency of transdifferentiation is increased by at least 49 fold. In some embodiments, efficiency of transdifferentiation is increased by at least 50 fold.

In some embodiments, a subject from which a non-neuronal cell, e.g., somatic cell are obtained is a mammalian subject, such a human subject. In some embodiments, the subject is suffering from a neurodegenerative disease, e.g., Alzheimer's disease, Parkinson's disease, multiple sclerosis, and the like. In some embodiments, the subject is suffering from a motor neuron disease, e.g., a amylotrophic lateral sclerosis (ALS), spinal muscular atrophy (SMA), primary lateral sclerosis (PLS), progressive bulbar palsy, pseudobulbar palsy, progressive muscular atrophy, post-polio syndrome (PPS) and the like. In such embodiments, the a non-neuronal cell, e.g., somatic cell can be converted into a iNs or iMNs ex vivo by the methods as described herein and then administered to the subject from which the cells were harvested in a method to treat the subject for the neurodegenerative disease or motor neuron disease or disorder.

In some embodiments, a non-neuronal cell, e.g., somatic cell is located within a subject (in vivo) and is directly converted to become an iN or iMN by the methods as disclosed herein in vivo. In some embodiments, direct conversion of a non-neuronal cell, e.g., somatic cell to a iN or iMN in vivo can be achieved by administering to a subject a composition comprising an agent which inhibits the level or activity of ALK4, ALK5, and ALK7. In some embodiments, direct conversion of a non-neuronal cell, e.g., somatic cell to a iN or iMN in vivo can be achieved by administering to a subject a composition comprising an agent which inhibits the level or activity of PLK1. In some embodiments, direct conversion of a non-neuronal cell, e.g., somatic cell to a iN or iMN in vivo can be achieved by administering to a subject a composition comprising an agent which inhibits the level or activity of ALK4, ALK5, ALK7 and PLK1. In some embodiments, direct conversion of a non-neuronal cell, e.g., somatic cell to a iN or MN in vivo can be achieved by transducing the non-neuronal cell, e.g., somatic cell with a viral vector, such as adenovirus which has the ability to express three or more MN-inducing agents selected from any combination of Lhx3, Ascl1, Brn2, Myt1l, Isl1, Hb9, Ngn2 or NeuroD1 in the somatic cell and administering to a subject a composition comprising an agent which inhibits the level or activity of ALK4, ALK5, and ALK7 in the subject. In some embodiments, direct conversion of a non-neuronal cell, e.g., somatic cell to a iN or MN in vivo can be achieved by transducing the fibroblast with a viral vector, such as adenovirus which has the ability to express three or more MN-inducing agents selected from any combination of Lhx3, Ascl1, Brn2, Myt1l, Isl1, Hb9, Ngn2 or NeuroD1 in the somatic cell and administering to a subject a composition comprising an agent which inhibits the level or activity of PLK1 in the subject. In some embodiments, direct conversion of a non-neuronal cell, e.g., somatic cell to a iN or MN in vivo can be achieved by transducing the non-neuronal cell, e.g., somatic cell with a viral vector, such as adenovirus which has the ability to express three or more MN-inducing agents selected from any combination of Lhx3, Ascl1, Brn2, Myt1l, Isl1, Hb9, Ngn2 or NeuroD1 in the somatic cell and administering to a subject a composition comprising an agent which inhibits the level or activity of ALK4, ALK5, ALK7 and PLK1 in the subject.

In some embodiments, such contacting may be performed by maintaining the non-neuronal cell, e.g., somatic cell in culture medium comprising the agent(s). In some embodiments a non-neuronal cell, e.g., somatic cell can be genetically engineered. In some embodiments, a non-neuronal cell, e.g., somatic cell can be genetically engineered to express one or more MN-inducing factors as disclosed herein, for example express at least one a polypeptide selected from Lhx3, Ascl1, Brn2, Myt1l, Isl1, Hb9, Ngn2 or NeuroD1, or an amino acid sequences substantially homologous thereof, or functional fragments or functional variants thereof.

Where the non-neuronal cell, e.g., somatic cell is maintained under in vitro conditions, conventional tissue culture conditions and methods can be used, and are known to those of skill in the art. Isolation and culture methods for various cells are well within the abilities of one skilled in the art.

In the methods of the disclosure a non-neuronal cell, e.g., somatic cell, e.g., fibroblast can, in general, be cultured under standard conditions of temperature, pH, and other environmental conditions, e.g., as adherent cells in tissue culture plates at 37° C. in an atmosphere containing 5-10% CO₂. The cells and/or the culture medium are appropriately modified to achieve direct conversion to iNs or iMNs as described herein. In certain embodiments, non-neuronal cell, e.g., somatic cell can be cultured on or in the presence of a material that mimics one or more features of the extracellular matrix or comprises one or more extracellular matrix or basement membrane components. In some embodiments Matrigel™ is used. Other materials include proteins or mixtures thereof such as gelatin, collagen, fibronectin, etc. In certain embodiments of the invention, a non-neuronal cell, e.g., somatic cell can be cultured in the presence of a feeder layer of cells. Such cells may, for example, be of murine or human origin. They can also be irradiated, chemically inactivated by treatment with a chemical inactivator such as mitomycin e, or otherwise treated to inhibit their proliferation if desired. In other embodiments a non-neuronal cell, e.g., somatic cell are cultured without feeder cells.

Methods of Transdifferentiation of Somatic Cells to iMNs or iNs

Generating iN or iMN by direct conversion of a non-neuronal cell, e.g., somatic cell using the methods of the disclosure has a number of advantages. First, the methods of the disclosure allow one to generate autologous iNs or iMNs, which are cells specific to and genetically matched with an individual. The cells are derived from a non-neuronal cell, e.g., somatic cell, e.g., fibroblast obtained from the individual. In general, autologous cells are less likely than non-autologous cells to be subject to immunological rejection.

Second, the methods of the disclosure allow the artisan to generate iNs or iMNs without using embryos, oocytes, and/or nuclear transfer technology. Herein, the applicants' results demonstrate that a non-neuronal cell, e.g., somatic cell can be directly converted to become a neuron (iN) or motor neuron (iMN), without the need to be fully reprogrammed to a pluripotent state, therefore minimizing the risk of differentiation into unwanted cell types or risk of teratomas formation.

Also encompassed in the methods of the disclosure is a method of conversion of a non-neuronal cell, e.g., somatic cell, e.g., fibroblast by means other than engineering the cells to express MN-inducing factors, i.e., by contacting a non-neuronal cell, e.g., somatic cell, e.g., fibroblast with a MN-inducing factors other than a nucleic acid or viral vector capable of being taken up and causing a stable genetic modification to the cells. In particular, the invention encompasses the recognition that extracellular signaling molecules, e.g., molecules that when present extracellularly bind to cell surface receptors and activate intracellular signal transduction cascades, are of use to reprogram non-neuronal cell, e.g., somatic cells. The invention further encompasses the recognition that activation of such signaling pathways by means other than the application of extracellular signaling molecules is also of use to directly convert a non-neuronal cell, e.g., somatic cell, e.g., fibroblast into a iN or iMN. In addition, the methods of the disclosure relate to methods of identification of the iNs or iMNs that are detectable based on morphological criteria, without the need to employ a selectable marker, as well as functional characteristics, such as ability to generate action potentials, resting membrane potential of less than −50 mV, responsive to inhibitory neurotransmitters such as glycine and GABA, and responsiveness to excitatory neurotransmitters such as glutamate. The present disclosure thus reflects several fundamentally important advances in the area of somatic cell transdifferentiation technology, in particular direct conversion of non-neuronal cell, e.g., somatic cell to neurons, for example a subtype of neurons, in particular, motor neurons.

While certain aspects of the invention are exemplified herein using RepSox and BI 2536, the methods of the invention encompass use of any other agents which inhibit the level or activity of ALK4, ALK5, and ALK7 or PLK1, respectively, in replace of RepSox and BI 2536, where the other agents (e.g., MN-inducing factors) includes, for example, but is not limited to, Oligo2, Pax6, Sox1, Nkx6.1 or functional variants, homologues or functional fragments thereof for the purposes of converting a somatic cell, e.g., fibroblast to iN or iMN.

While certain aspects of the invention are exemplified herein using at least three different MN-inducing factors, e.g., Lhx3, Ascl1, Brn2, Myt1l, Isl1, Hb9, Ngn2, NeuroD1, the methods of the invention encompass use of any other MN-inducing factors in replace of any one of Lhx3, Ascl1, Brn2, Myt1l, Isl1, Hb9, Ngn2 or NeuroD1, where the other MN-inducing factors includes, for example, but is not limited to, Oligo2, Pax6, Sox1, Nkx6.1 or functional variants, homologues or functional fragments thereof for the purposes of converting a non-neuronal cell, e.g., somatic cell, e.g., fibroblast to iN or iMN.

Another aspect of the disclosure relates to methods to produce a population of isolated iN or iMN by inhibiting the level or activity of ALK4, ALK5, and ALK7 in a population of non-neuronal cell, e.g., somatic cell, e.g., fibroblasts. In some embodiments, a non-neuronal cell, e.g., somatic cell, e.g., fibroblast can be treated in any of a variety of ways to cause direct conversion of the non-neuronal cell, e.g., somatic cell to an iN or iMN according to the methods of the disclosure. For example, in some embodiments, the treatment can comprise contacting the cells with one or more agent(s), herein referred to as a “ALK4, ALK5, ALK7 inhibiting agent” which decreases the level or activity of ALK4, ALK5, and ALK7 in the cells. In some embodiments, the method comprises converting a non-neuronal cell, e.g., somatic cell, e.g., fibroblast by decreasing the level or activity of ALK4, ALK5, and ALK7 in the non-neuronal cell, e.g., somatic cell (e.g., fibroblast) wherein the level or activity is decreased for sufficient amount of time to allow the conversion of the cell to become a cell which exhibits at least two characteristics of a endogenous neuron or motor neuron (e.g., a motor neuron differentiated from an embryonic stem cell), for example at least two of the following characteristics; (i) expression of motor neuron markers, for example, but not limited to P2-tubulins (e.g, Tubb2a and Tubb2b), Map2, synapsins (e.g., Syn1 and Syn2), synaptophysin, synaptotagmins (e.g., Syt1, Syt4, Syt13, Syt 16), NeuroD, Isl1, cholineacetyltransferase (ChAT), e.g., vescular ChAT, (ii) significantly decreased level of expression of non-neuronal cell, e.g., somatic cell, e.g., fibroblast genes from which they are derived, selected from the group of: Snail1, thy1 and Fsp1, (iii) exhibit typical motor neuron morphology, e.g., comprising a cell body with axonal projections which form functional synaptic junctions with muscle cells and (iv) an average resting potential of lower than about −50 mV, e.g., a resting potential of about −50 mV to about −65 mV and any integer between, e.g., about −50 mV, or about −50 to −55 mV or about −55 mV to about −60 mV or about −60 mV to about −65 mV, or alternatively a resting potential substantially the same as the resting membrane potential of motor neurons differentiated from embryonic stem cells (v) functional motor neuron characteristics selected from (a) the ability to fire action potentials, (b) responsiveness to inhibitory neurotransmitters glycine and GABA, and (c) responsiveness to excitatory neurotransmitters, e.g., glutamate or kainate.

Another aspect of the disclosure relates to methods to produce a population of isolated iN or iMN by inhibiting the level or activity of PLK1 in a population of non-neuronal cell, e.g., somatic cell, e.g., fibroblasts. In some embodiments, a non-neuronal cell, e.g., somatic cell, e.g., fibroblast can be treated in any of a variety of ways to cause direct conversion of the non-neuronal cell, e.g., somatic cell to an iN or iMN according to the methods of the disclosure. For example, in some embodiments, the treatment can comprise contacting the cells with one or more agent(s), herein referred to as a “PLK1 inhibiting agent” which decreases the level or activity of PLK1 in the cells. In some embodiments, the method comprises converting a non-neuronal cell, e.g., somatic cell, e.g., fibroblast by decreasing the level or activity of PLK in the non-neuronal cell, e.g., somatic cell (e.g., fibroblast) wherein the level or activity is decreased for sufficient amount of time to allow the conversion of the cell to become a cell which exhibits at least two characteristics of a endogenous neuron or motor neuron (e.g., a motor neuron differentiated from an embryonic stem cell), for example at least two of the following characteristics; (i) expression of motor neuron markers, for example, but not limited to P2-tubulins (e.g, Tubb2a and Tubb2b), Map2, synapsins (e.g., Syn1 and Syn2), synaptophysin, synaptotagmins (e.g., Syt1, Syt4, Syt13, Syt 16), NeuroD, Isl1, cholineacetyltransferase (ChAT), e.g., vescular ChAT, (ii) significantly decreased level of expression of non-neuronal cell, e.g., somatic cell, e.g., fibroblast genes from which they are derived, selected from the group of: Snail1, thy1 and Fsp1, (iii) exhibit typical motor neuron morphology, e.g., comprising a cell body with axonal projections which form functional synaptic junctions with muscle cells and (iv) an average resting potential of lower than about −50 mV, e.g., a resting potential of about −50 mV to about −65 mV and any integer between, e.g., about −50 mV, or about −50 to −55 mV or about −55 mV to about −60 mV or about −60 mV to about −65 mV, or alternatively a resting potential substantially the same as the resting membrane potential of motor neurons differentiated from embryonic stem cells (v) functional motor neuron characteristics selected from (a) the ability to fire action potentials, (b) responsiveness to inhibitory neurotransmitters glycine and GABA, and (c) responsiveness to excitatory neurotransmitters, e.g., glutamate or kainate.

Another aspect of the disclosure relates to methods to produce a population of isolated iN or iMN by inhibiting the level or activity of ALK4, ALK5, ALK7 and PLK1 in a population of non-neuronal cell, e.g., somatic cell, e.g., fibroblasts. In some embodiments, a non-neuronal cell, e.g., somatic cell, e.g., fibroblast can be treated in any of a variety of ways to cause direct conversion of the fibroblast to an iN or iMN according to the methods of the disclosure. For example, in some embodiments, the treatment can comprise contacting the cells with one or more ALK4, ALK5, ALK7 inhibiting agents and one or more PLK1 inhibiting agents which decrease the level or activity of ALK4, ALK5, and ALK7, and PLK1, respectively in the cells. In some embodiments, the method comprises converting a non-neuronal cell, e.g., somatic cell, e.g., fibroblast by decreasing the level or activity of ALK4, ALK5, ALK7 and PLK1 in the somatic cell (e.g., fibroblast) wherein the level or activity is decreased for sufficient amount of time to allow the conversion of the cell to become a cell which exhibits at least two characteristics of a endogenous neuron or motor neuron (e.g., a motor neuron differentiated from an embryonic stem cell), for example at least two of the following characteristics; (i) expression of motor neuron markers, for example, but not limited to P2-tubulins (e.g, Tubb2a and Tubb2b), Map2, synapsins (e.g., Syn1 and Syn2), synaptophysin, synaptotagmins (e.g., Syt1, Syt4, Syt13, Syt 16), NeuroD, Isl1, cholineacetyltransferase (ChAT), e.g., vescular ChAT, (ii) significantly decreased level of expression of fibroblast genes from which they are derived, selected from the group of: Snail1, thy1 and Fsp1, (iii) exhibit typical motor neuron morphology, e.g., comprising a cell body with axonal projections which form functional synaptic junctions with muscle cells and (iv) an average resting potential of lower than about −50 mV, e.g., a resting potential of about −50 mV to about −65 mV and any integer between, e.g., about −50 mV, or about −50 to −55 mV or about −55 mV to about −60 mV or about −60 mV to about −65 mV, or alternatively a resting potential substantially the same as the resting membrane potential of motor neurons differentiated from embryonic stem cells (v) functional motor neuron characteristics selected from (a) the ability to fire action potentials, (b) responsiveness to inhibitory neurotransmitters glycine and GABA, and (c) responsiveness to excitatory neurotransmitters, e.g., glutamate or kainate.

Another aspect of the disclosure relates to methods to produce a population of isolated iN or iMN by decreasing the level or activity of ALK4, ALK5, and ALK7 and/or PLK1 alone or in combination with increasing the protein expression of at least three MN-inducing factors in a population of a somatic cell, e.g., fibroblast. In some embodiments, a somatic cell, e.g., fibroblast can be treated in any of a variety of ways to cause direct conversion of the fibroblast to an iN or iMN according to the methods of the disclosure. For example, in some embodiments, the treatment can further comprise contacting the cells with one or more agent(s), herein referred to as a “MN-inducing factor” which increases the protein expression of at least three of the transcription factors selected from Lhx3, Ascl1, Brn2, Myt1l, Isl1, Hb9, Ngn2 or NeuroD1, or increases the protein expression of a functional homologue or a functional fragment of at least three of any combination of Lhx3, Ascl1, Brn2, Myt1l, Isl1, Hb9, Ngn2 or NeuroD1, polypeptides in the somatic cell, e.g., fibroblast.

In some embodiments, the method comprises converting a somatic cell, e.g., fibroblast by increasing the protein expression of at least three in any combination of the following MN-inducing factors Lhx3, Ascl1, Brn2, Myt1l, Isl1, Hb9, Ngn2 or NeuroD1, in the somatic cell (e.g., fibroblast) wherein the expression is for sufficient amount of time, typically transient increase in expression, to allow the conversion of the cell to become a cell which exhibits at least two characteristics of a endogenous motor neuron (e.g., a motor neuron differentiated from an embryonic stem cell), for example at least two of the following characteristics; (i) expression of motor neuron markers, for example, but not limited to P2-tubulins (e.g, Tubb2a and Tubb2b), Map2, synapsins (e.g., Syn1 and Syn2), synaptophysin, synaptotagmins (e.g., Syt1, Syt4, Syt13, Syt 16), NeuroD, Isl1, cholineacetyltransferase (ChAT), e.g., vescular ChAT, (ii) significantly decreased level of expression of fibroblast genes from which they are derived, selected from the group of: Snail1, thy1 and Fsp1, (iii) exhibit typical motor neuron morphology, e.g., comprising a cell body with axonal projections which form functional synaptic junctions with muscle cells and (iv) an average resting potential of lower than about −50 mV, e.g., a resting potential of about −50 mV to about −65 mV and any integer between, e.g., about −50 mV, or about −50 to −55 mV or about −55 mV to about −60 mV or about −60 mV to about −65 mV, or alternatively a resting potential substantially the same as the resting membrane potential of motor neurons differentiated from embryonic stem cells (v) functional motor neuron characteristics selected from (a) the ability to fire action potentials, (b) responsiveness to inhibitory neurotransmitters glycine and GAB A, and (c) responsiveness to excitatory neurotransmitters, e.g., glutamate or kainate.

In some embodiments, the method comprises reprogramming a somatic cell, e.g., fibroblast by increasing the protein expression of three or more of following MN-inducing transcription factors Lhx3, Ascl1, Brn2, Myt1l, Isl1, Hb9, Ngn2 or NeuroD1 in the somatic cell, e.g., fibroblast. The increase in expression of the transcription factors can be done all at the same time (e.g. concurrently), or alternatively, subsequently in any order.

In some embodiments, the method comprises reprogramming a somatic cell, e.g., fibroblast by expressing at least 2, or at least 3, or at least 4 or at least 5, or at least 6, or at least 7 or at least 8, or at least 9 or at least 10 or 11 of any combination of MN-inducing factors selected from, for example, but is not limited to, Lhx3, Ascl1, Brn2, Myt1l, Isl1, Hb9, Ngn2, NeuroD1 or functional variants, polypeptides with amino acids substantially homologues or functional fragments thereof in a somatic cell, e.g., fibroblast to reprogram to an iMN.

In some embodiments, increasing the protein expression can be by any means known by one of ordinary art, for example can include introduction of nucleic acid, or nucleic acid analogue encoding one or more of the MN-inducing factors, or contacting the somatic cell, e.g., fibroblast with an agent which converts the somatic cell, e.g., fibroblast to a cell with a motor neuron phenotype. In some embodiments, a nucleic acid analogue is a locked nucleic acid (LNA), or a modified synthetic RNA (modRNA) encoding one or more of the MN-inducing factors. ModRNA are well known by one of ordinary skill in the art, and are described in U.S. Provisional Application 61/387,220, filed Sep. 28, 2010, and U.S. Provisional Application 61/325,003, filed: Apr. 16, 2010, both of which are incorporated herein in their entirety by reference.

In some embodiments, a MN-inducing agent is a vector comprising a nucleotide sequence encoding the polypeptide one or more of Lhx3, Ascl1, Brn2, Myt1l, Isl1, Hb9, Ngn2, NeuroD or encoding a polypeptide substantially homologous or a functional variant or functional fragment of such polypeptides. In such embodiments, the nucleotide sequence can comprise any nucleic acid sequence selected from the nucleic acid sequences of Lhx3, Ascl1, Brn2, Myt1l, Isl1, Hb9, Ngn2, NeuroD or a fragment or variant thereof.

In some embodiments, the vector is a viral vector. In some embodiments, the viral vector is a non-integrating viral vector. While retroviral vectors incorporate into the host cell genome and can potentially disrupt normal gene function, non-integrating vectors have the advantage of controlling expression of a gene product by extra-chromosomal transcription. It follows that since non-integrating vectors do not become part of the host genome, non-integrating vectors tend to express a nucleic acid transiently in a cell population. This is due in part to the fact that the non-integrating vectors as used herein are rendered replication deficient. Thus, non-integrating vectors have several advantages over retroviral vectors including but not limited to: (1) no disruption of the host genome, and (2) transient expression, and (3) no remaining viral integration products.

Some non-limiting examples of non-integrating vectors include adenovirus, baculovirus, alphavirus, picornavirus, and vaccinia virus. In one embodiment, the non-integrating viral vector is an adenovirus. The advantages of non-integrating viral vectors further include the ability to produce them in high titers, their stability in vivo, and their efficient infection of host cells.

While it is known that some non-integrating vectors integrate into the host genome at extremely low frequencies (i.e., 10″4 to 10″5), a non-integrating vector, as the term is used herein, refers to vectors having a frequency of integration of less than 0.1% of the total number of infected cells; preferably the frequency of integration is less than 0.01%, less than 0.001%, less than 0.0001%, or less than 0.000001% (or lower) of the total number of infected cells. In one embodiment, the vector does not integrate at all. In another embodiment, the viral integration remnants of the virus are below the detection threshold as assayed by PCR (for nucleic acid detection) or immunoassay (for protein detection). In general, iNs or iMNs produced by the methods described herein should be assayed for an integration event by the viral vector using, for example, PCR-mediated detection of the viral genome prior to administering a population of iNs or iMNs to a subject. Any iN or iMN with detectable integration products should not be administered to a subject.

The viral titer necessary to achieve a desired (i.e., effective) level of gene expression in a host cell is dependent on many factors, including, for example, the cell type, gene product, culture conditions, co-infection with other viral vectors, and co-treatment with other agents, among others. It is well within the abilities of one skilled in the art to test a range of titers for each virus or combination of viruses by detecting the expression levels of either (a) a marker expression product, or (b) a test gene product. Detection of protein expression in cells can be achieved by several techniques including Western blot analysis, immuno-cytochemistry, and fluorescence-mediated detection, among others. It is contemplated that experiments are first optimized by testing a variety of titer ranges for each cell type under the desired culture conditions. Once an optimal titer of a virus or a cocktail of viruses is determined, then that protocol will be used to induce the reprogramming of somatic cells.

In addition to viral titers, it is also important that the infection and induction times are appropriate with respect to different cells. For example, as discussed in the Examples section herein, initial attempts with an adenoviral vector were deemed unsuccessful due to an inadequate induction time. Upon recognition of this important consideration and considerable lengthening of induction time, induced pluripotent stem cells were produced using an adenoviral vector. With the knowledge provided herein that length of time is an important variable in induced pluripotent stem cell induction, one of skill in the art can test a variety of time points for infection or induction using a non-integrating vector and recover induced pluripotent stem cells from a given somatic cell type.

In some embodiments, the vector is a non-viral polycystronic vector as disclosed in Gonzalez et al., Proc. Natl. Acad. Sci. USA 2009 106:8918-8922; Carey et al., PNAS, 2009; 106; 157-162, WO/2009/065618 and WO/2000/071096 and Okita et al., Science 7, 2008: 322; 949-953, which are all incorporated herein in their entirety by reference.

In some embodiments, the nucleic acid is a modified synthetic RNA (modRNA) encoding one or more of the MN-inducing factors. ModRNA are well known by one of ordinary skill in the art, and are described in U.S. Provisional Application 61/387,220, filed Sep. 28, 2010, and U.S. Provisional Application 61/325,003, filed: Apr. 16, 2010, both of which are incorporated herein in their entirety by reference.

In other embodiments, the methods or the disclosure encompass non-viral means to increase the expression of iMN inducing factors (e.g. Lhx3), Ascl1, Brn2, Myt1l, Isl1, Hb9, Ngn2 and/or NeuroD1 in a somatic cell, e.g., fibroblast for the purposes for converting to an iMN as disclosed herein. For example, in one embodiment, naked DNA technology can be used, for example nucleic acid encoding the polypeptides of least three transcription factors selected from Lhx3, Ascl1, Brn2, Myt1l, Isl1, Hb9, Ngn2 or NeuroD1 can be introduced into a somatic cell, e.g., fibroblast for the purposes of converting the cell to an iMN. Methods of naked DNA technology are well known in the art, and are disclosed in U.S. Pat. No. 6,265,387 (which is incorporated herein in its entirety by reference) which describes a method of delivering naked DNA into a hepatocyte in vivo the via bile duct. U.S. Pat. No. 6,372,722 (which is incorporated herein in its entirety by reference) describes a method of naked DNA delivery to a secretory gland cell, for example, a pancreatic cell, a mammary gland cell, a thyroid cell, a thymus cell, a pituitary gland cell, and a liver cell.

In some embodiments, another non-viral means to increase the expression of the transcription factors (e.g. Lhx3, Ascl1, Brn2, Myt1l, Isl1, Hb9, Ngn2 or NeuroD1), in a somatic cell, e.g., fibroblast include use of piggyBac transposon vectors, as disclosed in U.S. Pat. No. 7,129,083, and 6,551,825; U.S. Patent Application 2009/0042297 and International Patent Application WO/2007/100821 which are incorporated herein in their entirety by reference.

Other non-viral means to increase the expression of the transcription factors (e.g. Lhx3, Ascl1, Brn2, Myt1l, Isl1, Hb9, Ngn2 or NeuroD1), in a somatic cell, e.g., fibroblast for the purposes for transdifferentiation to a iMN are also encompassed for use in the methods as disclosed herein.

In one embodiment, one can contact the a somatic cell, e.g., fibroblast with polypeptides or peptides of Lhx3, Ascl1, Brn2, Myt1l, Isl1, Hb9, Ngn2 or NeuroD1 or functional variants, polypeptides with amino acids substantially homologues or functional fragments thereof in a somatic cell, e.g., fibroblast to convert to an iMN. Alternatively, one can use aptamers or antibodies or any other agent which activates and increases the expression of the transcription factors (e.g. Lhx3, Ascl1, Brn2, Myt1l, Isl1, Hb9, Ngn2 or NeuroD1) in a somatic cell, e.g., fibroblast.

In alternative embodiments, one can contact the somatic cell, e.g., fibroblast with a small molecule or combination of small molecules (e.g. chemical complementation) to increase the expression of at least two transcription factors in the somatic cell, e.g., fibroblast.

Thus, in some embodiments, the contacting step will typically be for at least twenty-four hours. By “at least twenty-four hours,” is meant twenty-four hours or greater. In some embodiments, fibroblast cells can be contacted with ALK4, ALK5, and ALK7 inhibiting agents (e.g. small molecule, polypeptide, nucleic acid, nucleic acid analogues, etc) for about 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 hours up to 3, 4, 5, 6, 7, or more days or any particular intervening time in hours or minutes within the above range. Preferably, somatic cells, e.g., fibroblasts can be contacted with a ALK4, ALK5 and ALK7 inhibiting agent for seven days. In some embodiments, fibroblast cells can be contacted with PLK1 inhibiting agents (e.g. small molecule, polypeptide, nucleic acid, nucleic acid analogues, etc.) for about 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 hours up to 3, 4, 5, 6, 7, or more days or any particular intervening time in hours or minutes within the above range. Preferably, somatic cells, e.g., fibroblasts can be contacted with a PLK1 inhibiting agent for seven days. In some embodiments, fibroblast cells can be contacted with ALK4, ALK5, ALK7 and PLK1 inhibiting agents (e.g. small molecule, polypeptide, nucleic acid, nucleic acid analogues, etc) for about 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 hours up to 3, 4, 5, 6, 7, or more days or any particular intervening time in hours or minutes within the above range. Preferably, somatic cells, e.g., fibroblasts can be contacted with a ALK4, ALK5, ALK7 and PLK1 inhibiting agent for seven days.

In some embodiments, fibroblast cells can be contacted with MN-inducing factor (e.g. small molecule, polypeptide, nucleic acid, nucleic acid analogues, etc) for about 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 hours up to 3, 4, 5, 6, 7, or more days or any particular intervening time in hours or minutes within the above range. Preferably, somatic cells, e.g., fibroblasts can be contacted with a MN-inducing agent for seven days.

In another embodiment, the disclosure provides a method of direct conversion of somatic cells, e.g., fibroblasts by contacting the somatic cell with at least 3 or more polypeptides selected from any combination from the group of Lhx3, Ascl1, Brn2, Myt1l, Isl1, Hb9, Ngn2 or NeuroD1, or having amino acid sequences substantially homologous thereto, and functional fragments or functional variants thereof. In some embodiments, the disclosure provides a method of reprogramming a somatic cell, e.g., fibroblast comprising contacting the somatic cell, e.g., fibroblast with at least 3 polypeptides selected from the group of polypeptides of Lhx3, Ascl1, Brn2, Myt1l, Isl1, Hb9, Ngn2 and NeuroD1, or having amino acid sequences substantially homologous thereto, and functional fragments or functional variants thereof.

Where the ALK4, ALK5, and ALK7 inhibiting agent, the PLK1 inhibiting agent, or the MN-inducing factor is a polypeptide, e.g. a polypeptide of Lhx3, Ascl1, Brn2, Myt1l, Isl1, Hb9, Ngn2 or NeuroD1, the dosages of Lhx3, Ascl1, Brn2, Myt1l, Isl1, Hb9, Ngn2 or NeuroD1 polypeptides, their active fragments or related growth factors to be used in the in vivo or in vitro methods and processes of the invention preferably range from about 1 pmoles/kg/minute to about 100 nmoles/kg/minute for continuous administration and from about 1 nmoles/kg to about 40 mmoles/kg for bolus injection. Preferably, the dosage of Lhx3, Ascl1, Brn2, Myt1l, Isl1, Hb9, Ngn2 or NeuroD polypeptides in in vitro methods will be 10 pmoles/kg/min to about 100 nmoles/kg/min, and in in vivo methods from about 0.003 nmoles/kg/min to about 48 nmoles/kg/min. More preferably, the dosage of Lhx3, Ascl1, Brn2, Myt1l, Isl1, Hb9, Ngn2 or NeuroD1 polypeptides in in vitro methods ranges from about 100 picomoles/kg/minute to about 10 nanomoles/kg/minute, and in in vivo methods from about 0.03 nanomoles/kg/minute to about 4.8 nanomoles/kg/minute. In some embodiments, the preferred dosage of Lhx3, Ascl1, Brn2, Myt1l, Isl1, Hb9, Ngn2 or NeuroD polypeptides in in vitro methods is 1 pmoles/kg/min to about 10 nmoles/kg/mine, and in in vivo from about 1 pmole/kg to about 400 pmoles/kg for a bolus injection. The more preferred dosage of the preferred dosage of Lhx3, Ascl1, Brn2, Myt1l, Isl1, Hb9, Ngn2 or NeuroD1 polypeptides in in vitro methods ranges from about 10 pmole/kg/minute to about 1 nmole/kg/minute, and in in vivo from about 10 pmoles/kg to about 40 pmoles/kg for a bolus injection.

Confirming Presence of a iMN

An iN or iMN as disclosed herein, produced by the methods as disclosed herein is a cell with the phenotypic characteristics of an endogenous motor neurons. An iN or iMN can have all the phenotypic and functional characteristics of an endogenous motor neuron or may have less than all the phenotypic and functional characteristics of an endogenous motor neuron.

In some embodiments, the iN or iMN can exhibit a neuron morphology (e.g., motor neuron morphology) but otherwise maintain at least one phenotypic characteristic of the somatic cell from which it as converted from. For example, in some embodiments, a somatic cell, e.g., fibroblast that is subjected to an decrease in the level or activity of ALK4, ALK5, and ALK7 and/or PLK1 as disclosed herein can continue to express Snail and other fibroblast markers, but, unlike the typical fibroblast, the iN cell or iMN cell also conducts action potentials and exhibits one or more functional characteristics of a neuron or motor neuron. Thus, a continuum between complete phenotypic change and a single phenotypic change is possible. An increase in proliferation of a somatic cell, e.g., fibroblast may precede the direct conversion to iN or iMNs, and “transdifferentiation” is not meant to exclude any proliferation that accompanies the change of the cell to a iN or iMN phenotype.

To confirm the transdifferentiation of a somatic cell, e.g., fibroblast to an iN or iMN, isolated clones can be tested for the expression of a marker of neurons or motor neurons, respectively. Such expression identifies the cells as a neuron or motor neuron. Markers for motor neurons (iMNs) can be selected from the non-limiting group including (β2-tubulins (e.g, Tubb2a and Tubb2b), Map2, synapsins (e.g., Syn1 and Syn2), synaptophysin, synaptotagmins (e.g., Syt1, Syt4, Syt13, Syt 16), NeuroD, Isl1, cholineacetyltransferase (ChAT), e.g., vescular ChAT (VChAT), immunostaining of a-BTX, where expression is by a statistically significant amount as compared to the somatic cell, e.g., fibroblast from which the iMN was converted from.

Methods for detecting the expression of such markers are well known in the art, and include, for example, RT-PCR and immunological methods that detect the presence of the encoded polypeptides, such as ELISA.

In some embodiments, an iN or iMNs produced by the methods as disclosed herein can be identified based on unique morphological characteristics. In some embodiments, the iMN have a large cell body and axonal projections which form synaptic connections with muscle. As disclosed herein, iMN can be co-cultured with muscle cells, e.g., myotubules or C2Cl2 muscle co-culture according to the methods disclosed in the Examples section of PCT International Publication No. WO2013/025963, and form axonal projections along the length of the myotubules, which undergo regular and rhythmic contractions due to the synaptic connections with the iMNs (see FIG. 41). Thus, in some embodiments, the iMN have a unique functional characteristics with muscle as compared to other non-motor neuron neuronal subtypes.

In some embodiments, the iMN can be identified based on an average resting potential of lower than about −50 mV, e.g., a resting potential of about −50 mV to about −65 mV and any integer between, e.g., about −50 mV, or about −50 to −55 mV or about −55 mV to about −60 mV or about −60 mV to about −65 mV, or alternatively a resting potential substantially the same as the resting membrane potential of motor neurons differentiated from embryonic stem cells. In some embodiments, a iMN can be identified based on functional motor neuron characteristics, such as, but not limited to (a) the ability to fire action potentials, (b) responsiveness to inhibitory neurotransmitters glycine and GABA, and (c) responsiveness to excitatory neurotransmitters, e.g., glutamate or kainate.

In some embodiments, the iMN has a cell body size between about 30-80 μm in diameter, for example, in some embodiments, the iMN are gamma MN and are about at least about 40 μm, or at least about 50 μm, or about at least 60 μm, or at least about 70 μm, or at least about 80 μm, or any integer between about 40-80 μm, and in some embodiments, the iMN is an alpha motor neuron, and has a cell body size of at least about 19 μm, or at least about 20 μm, or at least about 21 μm, or at least about 22 μm, or at least about 23 μm, or at least about 24 μm, or at least about 25 μm, or at least about 26 μm, or at least about 27 μm, or greater than about 30 μm in diameter, or any integer between about 15-35 μm in diameter.

In some embodiments, the disclosure relates to an isolated population of iN produced by the methods as disclosed herein. In some embodiments, iN can be isolated by methods known in the art, for example FACs sorting, as disclosed in Liu et al, Journal Sichuan University, medical science edition, 209; 40(1); 153-6 or Liu et al, J Biol Chem, 1998; 273, 22201-22208, which are incorporated herein by reference).

In some embodiments, the disclosure relates to an isolated population of iMN produced by the methods as disclosed herein. In some embodiments, iMN can be isolated by methods known in the art, for example FACs sorting, as disclosed in Liu et al, Journal Sichuan University, medical science edition, 209; 40(1); 153-6 or Liu et al, J Biol Chem, 1998; 273, 22201-22208, which are incorporated herein by reference).

Monitoring the Production of iNs or iMNs from a Somatic Cell, e.g., Fibroblast

The progression of a somatic cell, e.g., fibroblast to an iN can be monitored by determining the expression of markers characteristic of neurons. The progression of a somatic cell, e.g., fibroblast to an iMN can be monitored by determining the expression of markers characteristic of motor neurons. In some processes, the expression of certain markers is determined by detecting the presence or absence of the marker. Alternatively, the expression of certain markers can be determined by measuring the level at which the marker is present in the cells of the cell culture or cell population. In certain processes, the expression of markers characteristic of motor neurons as well as the lack of significant expression of markers characteristic of the somatic cell, e.g., fibroblast from which it was derived can readily be determined.

As described in connection with monitoring the production of an iN or iMN, qualitative or semiquantitative techniques, such as blot transfer methods and immunocytochemistry, can be used to measure marker expression. Alternatively, marker expression can be accurately quantitated through the use of technique such as Q-PCR. Additionally, it will be appreciated that many of the markers of iMNs are secreted compounds such as acetylcholine. As such, techniques for measuring extracellular motor neuron marker content include HPLC or ELISA or other methods commonly known by persons of ordinary skill in the art.

As will be appreciated by the skilled artisan, markers of motor neurons include the expression of markers, but are not limited to, 2-tubulins (e.g, Tubb2a and Tubb2b), Map2, synapsins (e.g., Syn1 and Syn2), synaptophysin, synaptotagmins (e.g., Syt1, Syt4, Syt13, Syt 16), NeuroD, Isl1, cholineacetyltransferase (ChAT), e.g., vascular ChAT (VChAT), immunostaining of OC-BTX.

The iMNs produced by the processes described herein express one or more of the above-listed markers, thereby producing the corresponding gene products. However, it will be appreciated that iMNs need not express all of the above-described markers. For example, iMNs converted from a somatic cell, e.g., fibroblast do not always express Isl1.

In some embodiments, the transition of a somatic cell, e.g., fibroblast to an iN or iMN can be validated by monitoring the decrease in expression of fibroblast markers, e.g., Snail1, Thy1 and Fsp1 while monitoring the increase in expression of one or more of neuron markers or motor neuron markers. In addition to monitor the increase and/or decrease in expression of one or more the above-described markers, in some processes, the expression of genes indicative motor neurons or other neuronal markers can also be monitored.

It will be appreciated that 2-tubulins (e.g, Tubb2a and Tubb2b), Map2, synapsins (e.g., Syn1 and Syn2), synaptophysin, synaptotagmins (e.g., Syt1, Syt4, Syt13, Syt 16), NeuroD, Isl1, cholineacetyltransferase (ChAT), e.g., vescular ChAT (VChAT) marker expression is induced over a range of different levels in iMN depending on the differentiation conditions. As such, in some embodiments described herein, the expression of these markers are similar to the levels of expression in motor neurons differentiated from embryonic stem cells, e.g., at least about 70%, or at least about 80% or at least about 90% or at least about 100% or more than 100% the level of the expression of these markers by ES-derived motor neurons.

Methods of Identifying Agents for Transdifferentiation of Somatic Cells to iNs or iMNs.

Another aspect of the disclosure relates to methods of identifying agents that alone or in combination with other agents convert a somatic cell, e.g., fibroblast to an iN or iMN. In some embodiments, the method includes contacting one or more a somatic cell, e.g., fibroblast with one or more test agents (simultaneously or at separate times) and determining the level or activity of ALK4, ALK5, and ALK7. Where one or more test agents that decreases the level or activity of ALK4, ALK5, and ALK7 below the level or activity of ALK4, ALK5, and ALK7 normally found in the somatic cell, in the absence of one or more test agents, are considered candidate agents to be used as ALK4, ALK5, and ALK7 inhibiting agents for transdifferentiation of a somatic cell, e.g., fibroblast to an iN or iMN. The test agents may include, but are not limited to, small molecules, nucleic acids, peptides, polypeptides, immunoglobulins, and oligosaccarides. In some embodiments, the just-mentioned method includes determining the level of expression of one or more of ALK4, ALK5, and ALK7. Expression levels can be determined by any means known by one of ordinary skill in the art, for example, by RT-PCR or immunological methods. In some embodiments, the just-mentioned method includes assaying for phosphorylation of a ALK4, ALK5, and ALK7 substrate.

In some embodiments, the method includes contacting one or more a somatic cell, e.g., fibroblast with one or more test agents (simultaneously or at separate times) and determining the level or activity of PLK1. Where one or more test agents that decreases the level or activity of PLK1 below the level or activity of PLK1 normally found in the somatic cell, in the absence of one or more test agents, are considered candidate agents to be used as PLK1 inhibiting agents for transdifferentiation of a somatic cell, e.g., fibroblast to an iN or iMN. The test agents may include, but are not limited to, small molecules, nucleic acids, peptides, polypeptides, immunoglobulins, and oligosaccarides. In some embodiments, the just-mentioned method includes determining the level of expression of one or more of PLK1. Expression levels can be determined by any means known by one of ordinary skill in the art, for example, by RT-PCR or immunological methods. In some embodiments, the just-mentioned method includes assaying for phosphorylation of a PLK1 substrate.

In some embodiments, the method includes contacting one or more a somatic cell, e.g., fibroblast with one or more test agents (simultaneously or at separate times) and determining the level or activity of ALK4, ALK5, ALK7, and PLK1. Where one or more test agents that decreases the level or activity of ALK4, ALK5, ALK7, and PLK1 below the level or activity of ALK4, ALK5, ALK7, and PLK1 normally found in the somatic cell, in the absence of one or more test agents, are considered candidate agents to be used as ALK4, ALK5, ALK7, and PLK1 inhibiting agents for transdifferentiation of a somatic cell, e.g., fibroblast to an iN or iMN. The test agents may include, but are not limited to, small molecules, nucleic acids, peptides, polypeptides, immunoglobulins, and oligosaccarides. In some embodiments, the just-mentioned method includes determining the level of expression of one or more of ALK4, ALK5, ALK7, and PLK1. Expression levels can be determined by any means known by one of ordinary skill in the art, for example, by RT-PCR or immunological methods. In some embodiments, the just-mentioned method includes assaying for phosphorylation of a ALK4, ALK5, ALK7, and PLK1 substrate.

In some embodiments, the method includes contacting one or more a somatic cell, e.g., fibroblast with one or more test agents (simultaneously or at separate times) and determining the level or activity of ALK4, ALK5, ALK7, and PLK1, along with the level of expression of one or more MN-inducing factors as defined herein. In some embodiments, the MN-inducing factors include any one of Lhx3, Ascl1, Brn2, Myt1l, Isl1, Hb9, Ngn2 or NeuroD1. Where one or more test agents that decreases the level or activity of ALK4, ALK5, ALK7, and PLK1 below the level or activity of ALK4, ALK5, ALK7, and PLK1 normally found in the somatic cell, and increase the level of expression of one or more of the foregoing genes above the level of expression normally found in the somatic cell, in the absence of one or more test agents, are considered candidate agents to be used as ALK4, ALK5, ALK7, and PLK1 inhibiting agents and MN-inducing agents for transdifferentiation of a somatic cell, e.g., fibroblast to an iN or iMN. Expression levels can be determined by any means known by one of ordinary skill in the art, for example, by RT-PCR or immunological methods.

Of particular interest are screening assays for agents that transdifferentiate a human somatic cell, e.g., fibroblast to a iN or iMN. A wide variety of assays may be used for this purpose, including immunoassays for protein binding; determination of cell growth, differentiation and functional activity; production of factors; and the like.

In the screening method of the invention for agents, the a somatic cell, e.g., fibroblast are contacted with the agent of interest, and the effect of the agent assessed by monitoring output parameters, such as the level or activity of ALK4, ALK5, and ALK7, and/or the level or activity of PLK1, and/or expression of MN-inducing factors such as, but not limited to Lhx3, Ascl1, Brn2, Myt1l, Isl1, Hb9, Ngn2 or NeuroD1, cell viability, motor neuron functional characteristics, and the like. The cells may be freshly isolated, cultured, genetically engineered as described above, or the like. The somatic cell, e.g., fibroblast may be environmentally induced variants of clonal cultures: e.g. split into independent cultures and grown under distinct conditions, for example with or without virus; in the presence or absence of other cytokines or combinations thereof. Alternatively, a somatic cell, e.g., fibroblast may be variants with a desired pathological characteristic. For example, the desired pathological characteristic includes a mutation and/or polymorphism which contribute to disease pathology.

In alternative embodiments, the methods of the invention can be used to screen for agents in which a somatic cell, e.g., fibroblast comprising a particular mutation and/or polymorphism respond differently compared with a somatic cell, e.g., fibroblast without the mutation and/or polymorphism, therefore the methods can be used for example, to asses an effect of a particular drug and/or agent on iNs or iMNs from a defined subpopulation of people and/or cells, therefore acting as a high-throughput screen for personalized medicine and/or pharmacogenetics. The manner in which cells respond to an agent, particularly a pharmacologic agent, including the timing of responses, is an important reflection of the physiologic state of the cell. Accordingly, the iMNs generated from human fibroblasts can be useful to study disease mechanisms due to different mutations for ALS and SMA, as well as to identify agents or therapeutic treatment to treat motor neuron diseases of different genetic ALS and SMA phenotypes, as well iMNs from subjects where the complex genetic variation resulting in the motor neuron disease is not yet known. Additionally, the iNs generated from human fibroblasts can be useful to study disease mechanisms due to different mutations for neurodegenerative disorders, as well as to identify agents or therapeutic treatment to treat neurodegenerative disorders of different genetic phenotypes, as well iNs from subjects where the complex genetic variation resulting in the neurodegenerative disorder is not yet known.

The agent used in the screening method can be selected from a group of a chemical, small molecule, chemical entity, nucleic acid sequences, an action; nucleic acid analogues or protein or polypeptide or analogue of fragment thereof. In some embodiments, the nucleic acid is DNA or RNA, and nucleic acid analogues, for example can be PNA, pcPNA and LNA. A nucleic acid may be single or double stranded, and can be selected from a group comprising; nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide agent or fragment thereof, can be any protein of interest, for example, but not limited to; mutated proteins; therapeutic proteins; truncated proteins, wherein the protein is normally absent or expressed at lower levels in the cell. Proteins of interest can be selected from a group comprising; mutated proteins, genetically engineered proteins, peptides, synthetic peptides, recombinant proteins, chimeric proteins, antibodies, humanized proteins, humanized antibodies, chimeric antibodies, modified proteins and fragments thereof. The agent may be applied to the media, where it contacts the cell (such as a somatic cell, e.g., fibroblast) and induces its effects. Alternatively, the agent may be intracellular within the cell (e.g. a somatic cell, e.g., fibroblast) as a result of introduction of the nucleic acid sequence into the cell and its transcription resulting in the production of the nucleic acid and/or protein agent within the cell. An agent also encompasses any action and/or event the cells (e.g. a somatic cell, e.g., fibroblast) are subjected to. As a non-limiting examples, an action can comprise any action that triggers a physiological change in the cell, for example but not limited to; heat-shock, ionizing irradiation, cold-shock, electrical impulse, light and/or wavelength exposure, UV exposure, pressure, stretching action, increased and/or decreased oxygen exposure, exposure to reactive oxygen species (ROS), ischemic conditions, fluorescence exposure etc. Environmental stimuli also include intrinsic environmental stimuli defined below. The exposure to agent may be continuous or non-continuous.

In some embodiments, the agent is an agent of interest including known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like. Candidate agents also include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Also included as agents are pharmacologically active drugs, genetically active molecules, etc.

Compounds of interest include, for example, chemotherapeutic agents, hormones or hormone antagonists, growth factors or recombinant growth factors and fragments and variants thereof. Exemplary pharmaceutical agents suitable for this invention are those described in, “The Pharmacological Basis of Therapeutics,” Goodman and Oilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

The agents include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

Parameters are quantifiable components of a somatic cell (e.g., fibroblast) particularly the level or activity of ALK4, ALK5, and ALK7, and/or PLK1. In some embodiments, the parameters include level or activity of one or more of ALK4, ALK5, ALK7 and PLK1 in any combination that can be accurately measured, desirably in a high throughput system. In some embodiments, a high throughput screen for resting membrane potential and responsiveness to inhibitory neurotransmitters, such as GABA and glycine, and excitatory neurotransmitters, such as glutamate can be used to identify an agent which induces transdifferentiation of a fibroblast into a functional iMN. In some embodiments, a secondary screen can be used to assess the functional characteristics if the iMN, e.g., ability to form synaptic junctions with muscle cells, as well as expression of motor neuron markers, for example, but not limited to, expression of 2-tubulins (e.g, Tubb2a and Tubb2b), Map2, synapsins (e.g., Syn1 and Syn2), synaptophysin, synaptotagmins (e.g., Syt1, Syt4, Syt13, Syt 16), NeuroD, Isl1, cholineacetyltransferase (ChAT), e.g., vescular ChAT (VChAT), immunostaining of OC-BTX. In some embodiments, the iMNs may express transcription factors specifically expressed in motor neurons, including Lim3, and HoxB1, HoxB6, HoxC5 and HoxC8, but not other neuronal markers of non-motor neuron subtypes. For instance, iMNs can be identified by lack of expression of forebrain neuronal markers, Otx2 and Bf-1, or mid-brain markers, En-1.

Parameters are quantifiable components of a somatic cell (e.g., fibroblast) particularly the expression of genes (e.g., protein expression or mRNA expression) such as, one or more in any combination of Lhx3, Ascl1, Brn2, Myt1l, Isl1, Hb9, Ngn2 or NeuroD1. In some embodiments, expression of one or more, in any combination of Lhx3, Ascl1, Brn2, Myt1l, Isl1, Hb9, Ngn2 or NeuroD1 that can be accurately measured, desirably in a high throughput system. In some embodiments, a high throughput screen for resting membrane potential and responsiveness to inhibitory neurotransmitters, such as GABA and glycine, and excitatory neurotransmitters, such as glutamate can be used to identify an agent which induces transdifferentiation of a fibroblast into a functional iMN. In some embodiments, a secondary screen can be used to assess the functional characteristics if the iMN, e.g., ability to form synaptic junctions with muscle cells, as well as expression of motor neuron markers, for example, but not limited to, expression of 2-tubulins (e.g, Tubb2a and Tubb2b), Map2, synapsins (e.g., Syn1 and Syn2), synaptophysin, synaptotagmins (e.g., Syt1, Syt4, Syt13, Syt 16), NeuroD, Isl1, cholineacetyltransferase (ChAT), e.g., vescular ChAT (VChAT), immunostaining of OC-BTX. In some embodiments, the iMNs may express transcription factors specifically expressed in motor neurons, including Lim3, and HoxB1, HoxB6, HoxC5 and HoxC8, but not other neuronal markers of non-motor neuron subtypes. For instance, iMNs can be identified by lack of expression of forebrain neuronal markers, Otx2 and Bf-1, or mid-brain markers, En-1.

In some embodiments, an output parameter from the screen can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values. In some embodiments, the assay is a computerized assay or a robotic high-throughput system operated through a computer interface.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for effect on a somatic cell, e.g., fibroblast by adding the agent to at least one and usually a plurality of a somatic cells, e.g., a population of fibroblasts, and can be performed concurrently with a test well with a somatic cell, e.g., fibroblast lacking the agent (e.g., reference culture). The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method. In some embodiments, agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Optionally, a somatic cell, e.g., fibroblast used in the screen can be manipulated to express desired gene products. Gene therapy can be used to either modify a cell to replace a gene product or add or knockdown a gene product. In some embodiments the genetic engineering is done to facilitate regeneration of tissue, to treat disease, or to improve survival of the iN or iMN following implantation into a subject (i.e. prevent rejection). Techniques for transfecting cells are known in the art.

A skilled artisan could envision a multitude of genes which would convey beneficial properties to a iN or iMN cell or, more indirectly, to a somatic cell, e.g., fibroblast used for transdifferentiation. The added gene may ultimately remain in the recipient cell and all its progeny, or may only remain transiently, depending on the embodiment. For example, genes encoding wild-type SOD1 could be transfected into a somatic cell, e.g., fibroblast. Such genes would be useful for producing iMNs with functional SOD1 protein where the fibroblast was obtained from a subject with an ALS-causing SOD1 mutation. In some situations, it may be desirable to transfect the cell with more than one gene.

In some instances, it is desirable to have the gene product secreted. In such cases, the gene product preferably contains a secretory signal sequence that facilitates secretion of the protein. For example, if the desired gene product is an angiogenic protein, a skilled artisan could either select an angiogenic protein with a native signal sequence, e.g. VEGF, or can modify the gene product to contain such a sequence using routine genetic manipulation (See Nabel et al., 1993).

The desired gene can be transfected into the cell using a variety of techniques. Preferably, the gene is transfected into the cell using an expression vector. Suitable expression vectors include plasmid vectors (such as those available from Stratagene, Madison Wis.), viral vectors (such as replication defective retroviral vectors, herpes virus, adenovirus, adeno-virus associated virus, and lentivirus), and non-viral vectors (such as liposomes or receptor ligands).

The desired gene is usually operably linked to its own promoter or to a foreign promoter which, in either case, mediates transcription of the gene product. Promoters are chosen based on their ability to drive expression in restricted or in general tissue types, for example in a somatic cell (e.g., fibroblast) or on the level of expression they promote, or how they respond to added chemicals, drugs or hormones. Other genetic regulatory sequences that alter expression of a gene may be co-transfected. In some embodiments, the host cell DNA may provide the promoter and/or additional regulatory sequences. Other elements that can enhance expression can also be included such as an enhancer or a system that results in high levels of expression.

Methods of targeting genes in mammalian cells are well known to those of skill in the art (U.S. Pat. Nos. 5,830,698; 5,789,215; 5,721,367 and 5,612,205). By “targeting genes” it is meant that the entire or a portion of a gene residing in the chromosome of a cell is replaced by a heterologous nucleotide fragment. The fragment may contain primarily the targeted gene sequence with specific mutations to the gene or may contain a second gene. The second gene may be operably linked to a promoter or may be dependent for transcription on a promoter contained within the genome of the cell. In a preferred embodiment, the second gene confers resistance to a compound that is toxic to cells lacking the gene. Such genes are typically referred to as antibiotic-resistance genes. Cells containing the gene may then be selected for by culturing the cells in the presence of the toxic compound.

Enrichment, Isolation and/or Purification of a Population of iNs or iMNs.

Another aspect of the disclosure relates to the isolation of a population of iN or iMN from a heterogeneous population of cells, such a comprising a mixed population of iN or iMN and somatic cells from which the iNs or iMNs were derived. A population of iN or iMN produced by any of the above-described processes can be enriched, isolated and/or purified by using an affinity tag that is specific for such cells. Examples of affinity tags specific for iN or iMN are antibodies, ligands or other binding agents that are specific to a marker molecule, such as a polypeptide, that is present on the cell surface of iN or iMN but which is not substantially present on other cell types (i.e. on the a somatic cell, e.g., fibroblast) that would be found in the heterogeneous population of cells produced by the methods described herein. In some processes, an antibody which binds to a cell surface antigen on human iN or iMN is used as an affinity tag for the enrichment, isolation or purification of iN or iMN produced by in vitro methods, such as the methods described herein. Such antibodies are known and commercially available.

The skilled artisan will readily appreciate that the processes for making and using antibodies for the enrichment, isolation and/or purification of iN or iMN are also readily adaptable for the enrichment, isolation and/or purification of iN or iMN. For example, analyzing and sorting for iNs or iMNs using a fluorescence activated cell sorter (FACS). Antibody-bound, fluorescent cells are collected separately from non-bound, non-fluorescent, thereby resulting in the isolation of such cell types.

In preferred embodiments of the processes described herein, the isolated cell composition comprising iN or iMN can be further purified by using an alternate affinity-based method or by additional rounds of sorting using the same or different markers that are specific for iN or iMN.

In some embodiments of the processes described herein, iN or iMN are fluorescently labeled without the use of an antibody then isolated from non-labeled cells by using a fluorescence activated cell sorter (FACS). In such embodiments, a nucleic acid encoding GFP, RFP, YFP or another nucleic acid encoding an expressible fluorescent marker gene, such as the gene encoding luciferase, is used to label iN or iMN using the methods described above, and as disclose in the Examples, where GFP is expressed in HB9 expressing cell. For example, in some embodiments, at least one copy of a nucleic acid encoding GFP or a biologically active fragment thereof is introduced into a somatic cell (e.g., fibroblast) preferably a human somatic cell (e.g., fibroblast) downstream of the HB9 promoter such that the expression of the GFP gene product or biologically active fragment thereof is under control of the HB9 promoter. In some embodiments, the entire coding region of the nucleic acid, which encodes HB9, is replaced by a nucleic acid encoding GFP or a biologically active fragment thereof. In other embodiments, the nucleic acid encoding GFP or a biologically active fragment thereof is fused in frame with at least a portion of the nucleic acid encoding HB9, thereby generating a fusion protein. In such embodiments, the fusion protein retains a fluorescent activity similar to GFP.

It will be appreciated that promoters other than the HB9 promoter can be used provided that the promoter corresponds to a marker that is expressed in motor neurons.

Fluorescently marked cells, such as the above-described a somatic cell (e.g., fibroblast) are differentiated to neurons or motor neurons as described previously above. Because iN or iMN express the fluorescent marker gene, whereas other cell types do not, iN or iMN can be separated from the other cell types. In some embodiments, cell suspensions comprising a population of a mixture of fluorescently-labeled iN or iMN and unlabeled non-iNs or non-iMNs (i.e. somatic cells, e.g., fibroblast from which the iNs or iMNs were derived) are sorted using a FACS. iNs or iMNs can be collected separately from non-fluorescing cells, thereby resulting in the isolation of iNs or iMNs. If desired, the isolated cell compositions comprising iNs or iMNs can be further purified by additional rounds of sorting using the same or different markers that are specific for neurons or motor neurons, respectively.

In preferred processes, iNs or iMNs are enriched, isolated and/or purified from other non-iNs or non-iMNs (i.e. from a somatic cell, e.g., fibroblast which have not been reprogrammed to become iNs or iMNs) after the cell population is induced to reprogram towards motor neurons using the methods and compositions as disclosed herein.

In addition to the procedures just described, iNs or iMNs may also be isolated by other techniques for cell isolation. Additionally, iNs or iMNs may also be enriched or isolated by methods of serial subculture in growth conditions which promote the selective survival or selective expansion of iNs or iMNs.

Using the methods described herein, enriched, isolated and/or purified populations of iNs or iMNs cells can be produced in vitro from a somatic cell (e.g., fibroblast) which has undergone sufficient transdifferentiation to produce at least some iNs or iMNs. In a preferred method, a population of somatic cells, e.g., fibroblasts can be transdifferentiated primarily into a population of iNs, where only a portion of the somatic cell population, e.g., about 5-10% has converted to iNs. Some preferred enrichment, isolation and/or purification methods relate to the in vitro production of iNs from human a somatic cell, e.g., fibroblast. In an alternative preferred method, a population of somatic cells, e.g., fibroblasts can be transdifferentiated primarily into a population of iMNs, where only a portion of the somatic cell population, e.g., about 5-10% has converted to iMNs. Some preferred enrichment, isolation and/or purification methods relate to the in vitro production of iMNs from human a somatic cell, e.g., fibroblast.

Using the methods described herein, isolated cell populations of iNs are enriched in iNs content by at least about 2- to about 1000-fold as compared to a population before transdifferentiation of the a somatic cell, e.g., fibroblast. In some embodiments, iNs can be enriched by at least about 5- to about 500-fold as compared to a population before transdifferentiation of the a somatic cell, e.g., fibroblast. In other embodiments, iNs can be enriched from at least about 10- to about 200-fold as compared to a population before transdifferentiation of the a somatic cell, e.g., fibroblast. In still other embodiments, iNs can be enriched from at least about 20- to about 100-fold as compared to a population before transdifferentiation of the a somatic cell, e.g., fibroblast. In yet other embodiments, iNs can be enriched from at least about 40- to about 80-fold as compared to a population before transdifferentiation of the a somatic cell, e.g., fibroblast. In certain embodiments, iNs can be enriched from at least about 2- to about 20-fold as compared to a population before transdifferentiation of the a somatic cell, e.g., fibroblast.

Using the methods described herein, isolated cell populations of iMNs are enriched in iMNs content by at least about 2- to about 1000-fold as compared to a population before transdifferentiation of the a somatic cell, e.g., fibroblast. In some embodiments, iMNs can be enriched by at least about 5- to about 500-fold as compared to a population before transdifferentiation of the a somatic cell, e.g., fibroblast. In other embodiments, iMNs can be enriched from at least about 10- to about 200-fold as compared to a population before transdifferentiation of the a somatic cell, e.g., fibroblast. In still other embodiments, iMNs can be enriched from at least about 20- to about 100-fold as compared to a population before transdifferentiation of the a somatic cell, e.g., fibroblast. In yet other embodiments, iMNs can be enriched from at least about 40- to about 80-fold as compared to a population before transdifferentiation of the a somatic cell, e.g., fibroblast. In certain embodiments, iMNs can be enriched from at least about 2- to about 20-fold as compared to a population before transdifferentiation of the a somatic cell, e.g., fibroblast.

Compositions Comprising iNs or iMNs

Some embodiments of the disclosure relate to cell compositions, such as cell cultures or cell populations, comprising iNs, wherein the iNs are neurons which have been derived from cells e.g. human a somatic cell (e.g., fibroblast) which express or exhibit one or more characteristics of an endogenous neuron. In accordance with certain embodiments, the iNs are mammalian cells, and in a preferred embodiment, such cells are human iNs.

Some embodiments of the disclosure relate to cell compositions, such as cell cultures or cell populations, comprising iMNs, wherein the iMNs are motor neurons which have been derived from cells e.g. human a somatic cell (e.g., fibroblast) which express or exhibit one or more characteristics of an endogenous motor neuron. In accordance with certain embodiments, the iMNs are mammalian cells, and in a preferred embodiment, such cells are human iMNs.

Other embodiments of the disclosure relate to compositions, such as cell cultures or cell populations, comprising iNs. In such embodiments, somatic cells, e.g., fibroblasts comprise less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 12%, less than about 10%, less than about 8%, less than about 6%, less than about 5%, less than about 4%, less than about 3%, less than about 2% or less than about 1% of the total cells in the cell population.

Other embodiments of the disclosure relate to compositions, such as cell cultures or cell populations, comprising iMNs. In such embodiments, somatic cells, e.g., fibroblasts comprise less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 12%, less than about 10%, less than about 8%, less than about 6%, less than about 5%, less than about 4%, less than about 3%, less than about 2% or less than about 1% of the total cells in the cell population.

Certain other embodiments of the disclosure relate to compositions, such as cell cultures or cell populations, comprising iNs. In some embodiments, a somatic cell, e.g., fibroblast from which the iNs are derived comprise less than about 25%, less than about 20%, less than about 15%, less than about 10%, less than about 5%, less than about 4%, less than about 3%, less than about 2% or less than about 1% of the total cells in the culture. In certain embodiments, iNs comprise less than about 25%, less than about 20%, less than about 15%, less than about 10%, less than about 5%, less than about 4%, less than about 3%, less than about 2% or less than about 1% of the total cells in the culture.

Certain other embodiments of the disclosure relate to compositions, such as cell cultures or cell populations, comprising iMNs. In some embodiments, a somatic cell, e.g., fibroblast from which the iMNs are derived comprise less than about 25%, less than about 20%, less than about 15%, less than about 10%, less than about 5%, less than about 4%, less than about 3%, less than about 2% or less than about 1% of the total cells in the culture. In certain embodiments, iMNs comprise less than about 25%, less than about 20%, less than about 15%, less than about 10%, less than about 5%, less than about 4%, less than about 3%, less than about 2% or less than about 1% of the total cells in the culture.

Additional embodiments of the disclosure relate to compositions, such as cell cultures or cell populations, produced by the processes described herein and which comprise iNs as the majority cell type. In some embodiments, the processes described herein produce cell cultures and/or cell populations comprising at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 81%, at least about 80%, at least about 79%, at least about 78%, at least about 77%, at least about 76%, at least about 75%, at least about 74%, at least about 73%, at least about 72%, at least about 71%, at least about 70%, at least about 69%, at least about 68%, at least about 67%, at least about 66%, at least about 65%, at least about 64%, at least about 63%, at least about 62%, at least about 61%, at least about 60%, at least about 59%, at least about 58%, at least about 57%, at least about 56%, at least about 55%, at least about 54%, at least about 53%, at least about 52%, at least about 51% or at least about 50% iNs. In preferred embodiments, the cells of the cell cultures or cell populations comprise human cells. In other embodiments, the processes described herein produce cell cultures or cell populations comprising at least about 50%, at least about 45%, at least about 40%, at least about 35%, at least about 30%, at least about 25%, at least about 24%, at least about 23%, at least about 22%, at least about 21%, at least about 20%, at least about 19%, at least about 18%, at least about 17%, at least about 16%, at least about 15%, at least about 14%, at least about 13%, at least about 12%, at least about 11%, at least about 10%, at least about 9%, at least about 8%, at least about 7%, at least about 6%, at least about 5%, at least about 4%, at least about 3%, at least about 2% or at least about 1% iNs. In preferred embodiments, the cells of the cell cultures or cell populations comprise human cells. In some embodiments, the percentage of iNs in the cell cultures or populations is calculated without regard to the feeder cells remaining in the culture.

Additional embodiments of the disclosure relate to compositions, such as cell cultures or cell populations, produced by the processes described herein and which comprise iMNs as the majority cell type. In some embodiments, the processes described herein produce cell cultures and/or cell populations comprising at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 81%, at least about 80%, at least about 79%, at least about 78%, at least about 77%, at least about 76%, at least about 75%, at least about 74%, at least about 73%, at least about 72%, at least about 71%, at least about 70%, at least about 69%, at least about 68%, at least about 67%, at least about 66%, at least about 65%, at least about 64%, at least about 63%, at least about 62%, at least about 61%, at least about 60%, at least about 59%, at least about 58%, at least about 57%, at least about 56%, at least about 55%, at least about 54%, at least about 53%, at least about 52%, at least about 51% or at least about 50% iMNs. In preferred embodiments, the cells of the cell cultures or cell populations comprise human cells. In other embodiments, the processes described herein produce cell cultures or cell populations comprising at least about 50%, at least about 45%, at least about 40%, at least about 35%, at least about 30%, at least about 25%, at least about 24%, at least about 23%, at least about 22%, at least about 21%, at least about 20%, at least about 19%, at least about 18%, at least about 17%, at least about 16%, at least about 15%, at least about 14%, at least about 13%, at least about 12%, at least about 11%, at least about 10%, at least about 9%, at least about 8%, at least about 7%, at least about 6%, at least about 5%, at least about 4%, at least about 3%, at least about 2% or at least about 1% iMNs. In preferred embodiments, the cells of the cell cultures or cell populations comprise human cells. In some embodiments, the percentage of iMNs in the cell cultures or populations is calculated without regard to the feeder cells remaining in the culture.

Still other embodiments of the disclosure relate to compositions, such as cell cultures or cell populations, comprising mixtures of iNs or iMNs and a somatic cell, e.g., fibroblast. For example, cell cultures or cell populations comprising at least about 5 iNs or iMNs for about every 95 somatic cells, e.g., fibroblast can be produced. In other embodiments, cell cultures or cell populations comprising at least about 95 iNs or iMNs for about every 5 somatic cell, e.g., fibroblast can be produced. Additionally, cell cultures or cell populations comprising other ratios of iNs or iMNs to somatic cell, e.g., fibroblast are contemplated. For example, compositions comprising at least about 1 iNs or iMNs for about every 1,000,000, or at least 100,000 cells, or a least 10,000 cells, or at least 1000 cells or 500, or at least 250 or at least 100 or at least 10 somatic cell, e.g., fibroblast. Further embodiments of the disclosure relate to compositions, such as cell cultures or cell populations, comprising human cells, including human iNs or iMNs.

In preferred embodiments of the disclosure, cell cultures and/or cell populations of iNs or iMNs comprise human iNs or iMNs that are non-recombinant cells. In such embodiments, the cell cultures and/or cell populations are devoid of or substantially free of recombinant human iNs or iMNs.

Using the processes described herein, compositions comprising iNs or iMNs are substantially free of other cell types can be produced. In some embodiments of the disclosure, the iNs or iMNs populations or cell cultures produced by the methods described herein are substantially free of cells that significantly express the fibroblast markers, or non-motor neuron markers.

Use of the iNs or iMNs

Another aspect of the disclosure further provides a method of treating a subject with a neurodegenerative disease or disorder, or treating a subject at risk of developing a neurodegenerative disease or disorder, comprising administering to the subject a composition comprising a population of iNs. Non-limiting examples of neurodegenerative disorders include polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, Guillain-Barré syndrome, ischemia stroke, Krabbe disease, kuru, Lewy body dementia, multiple sclerosis, multiple system atrophy, non-Huntingtonian type of Chorea, Parkinson's disease, Pelizaeus-Merzbacher disease, Pick's disease, primary lateral sclerosis, progressive supranuclear palsy, Refsum's disease, Sandhoff disease, Schilder's disease, spinal cord injury, spinal muscular atrophy (SMA), SteeleRichardson-Olszewski disease, and Tabes dorsalis.

Another aspect of the disclosure further provides a method of treating a subject with a motor neuron disease or disorder, or treating a subject at risk of developing a motor neuron disease or disorder, comprising administering to the subject a composition comprising a population of iMNs. In some embodiments the motor neuron disease or disorder is amyotrophic lateral sclerosis (ALS) or spinal muscular atrophy (SMA).

In some embodiments, the disclosure also provides a method of treating a motor neuron disease or disorder in a subject, comprising obtaining a population of somatic cells, e.g., fibroblasts from a subject, e.g. from the subject being treated, or from a donor subject; decreasing the level or activity of ALK4, ALK5, and ALK7 in the population of somatic cells, e.g., fibroblasts in vitro or ex vivo, for example by the methods as described herein, thereby promoting conversion of the population of somatic cells, e.g., fibroblasts into iMNs; and administering a substantially pure population of iMNs to the subject.

In some embodiments, the disclosure also provides a method of treating a motor neuron disease or disorder in a subject, comprising obtaining a population of somatic cells, e.g., fibroblasts from a subject, e.g. from the subject being treated, or from a donor subject; decreasing the level or activity of PLK1 in the population of somatic cells, e.g., fibroblasts in vitro or ex vivo, for example by the methods as described herein, thereby promoting conversion of the population of somatic cells, e.g., fibroblasts into iMNs; and administering a substantially pure population of iMNs to the subject.

In some embodiments, the disclosure also provides a method of treating a motor neuron disease or disorder in a subject, comprising obtaining a population of somatic cells, e.g., fibroblasts from a subject, e.g. from the subject being treated, or from a donor subject; decreasing the level or activity of ALK4, ALK5, ALK7, and PLK1 in the population of somatic cells, e.g., fibroblasts in vitro or ex vivo, for example by the methods as described herein, thereby promoting conversion of the population of somatic cells, e.g., fibroblasts into iMNs; and administering a substantially pure population of iMNs to the subject.

In some embodiments, the disclosure also provides a method of treating a motor neuron disease or disorder in a subject, comprising obtaining a population of somatic cells, e.g., fibroblasts from a subject, e.g. from the subject being treated, or from a donor subject; decreasing the level or activity of ALK4, ALK5, ALK7, and PLK1, together with increasing the protein expression of at least one transcription factors selected from Lhx3, Ascl1, Brn2, Myt1l, Isl1, Hb9, Ngn2 or NeuroD1 in the population of somatic cells, e.g., fibroblasts in vitro or ex vivo, for example by the methods as described herein, thereby promoting conversion of the population of somatic cells, e.g., fibroblasts into iMNs; and administering a substantially pure population of iMNs to the subject.

In some embodiments of the method of treating a motor neuron disease, a somatic cell, e.g., fibroblast can be from a donor, the donor can be a cadaver. As a further embodiment of the disclosure, a somatic cell, e.g., fibroblast can be allowed to proliferate in vitro or ex vivo prior to decreasing the level or activity of ALK4, ALK5, and ALK7. Preferably, promoting conversion of a somatic cell, e.g., fibroblast into iN or iMN as disclosed herein will result in greater than about 5% or about 10% of conversion of a somatic cell, e.g., fibroblast into iN or iMN. Even more preferably, greater than about 25%, 30%, 35%, 40%, 45%, 50%, 55%, 60%, 65%, 70%, 75%, 80%, 85%, or 90% of the a somatic cell, e.g., fibroblast will be converted into iN or iMNs.

In some embodiments of the method of treating a motor neuron disease, a somatic cell, e.g., fibroblast can be from a donor, the donor can be a cadaver. As a further embodiment of the disclosure, a somatic cell, e.g., fibroblast can be allowed to proliferate in vitro or ex vivo prior to decreasing the level or activity of PLK1. Preferably, promoting conversion of a somatic cell, e.g., fibroblast into iN or iMN as disclosed herein will result in greater than about 5% or about 10% of conversion of a somatic cell, e.g., fibroblast into iN or iMN. Even more preferably, greater than about 25%, 30%, 35%, 40%, 45%, 50%, 55%, 60%, 65%, 70%, 75%, 80%, 85%, or 90% of the a somatic cell, e.g., fibroblast will be converted into iN or iMNs.

In some embodiments of the method of treating a motor neuron disease, a somatic cell, e.g., fibroblast can be from a donor, the donor can be a cadaver. As a further embodiment of the disclosure, a somatic cell, e.g., fibroblast can be allowed to proliferate in vitro or ex vivo prior to decreasing the level or activity of ALK4, ALK5, ALK7, and PLK1. Preferably, promoting conversion of a somatic cell, e.g., fibroblast into iN or iMN as disclosed herein will result in greater than about 5% or about 10% of conversion of a somatic cell, e.g., fibroblast into iN or iMN. Even more preferably, greater than about 25%, 30%, 35%, 40%, 45%, 50%, 55%, 60%, 65%, 70%, 75%, 80%, 85%, or 90% of the a somatic cell, e.g., fibroblast will be converted into iN or iMNs.

In some embodiments of the method of treating a motor neuron disease, a somatic cell, e.g., fibroblast can be from a donor, the donor can be a cadaver. As a further embodiment of the disclosure, a somatic cell, e.g., fibroblast can be allowed to proliferate in vitro or ex vivo prior to decreasing the level or activity of ALK4, ALK5, ALK7, and PLK1 or increasing the protein expression of at least three or more MN-inducing factors selected from any combination of Lhx3, Ascl1, Brn2, Myt1l, Isl1, Hb9, Ngn2 or NeuroD1. Preferably, promoting conversion of a somatic cell, e.g., fibroblast into iMN as disclosed herein will result in greater than about 5% or about 10% of conversion of a somatic cell, e.g., fibroblast into iMN. Even more preferably, greater than about 25%, 30%, 35%, 40%, 45%, 50%, 55%, 60%, 65%, 70%, 75%, 80%, 85%, or 90% of the a somatic cell, e.g., fibroblast will be converted into iMNs.

In some embodiments, the iNs as disclosed herein can be used in cellular models of human neurodegenerative diseases, where such models could be used for basic research and drug discovery, e.g., to find treatments for neurodegenerative diseases or disorders including but not limited to polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, Guillain-Barré syndrome, ischemia stroke, Krabbe disease, kuru, Lewy body dementia, multiple sclerosis, multiple system atrophy, non-Huntingtonian type of Chorea, Parkinson's disease, Pelizaeus-Merzbacher disease, Pick's disease, primary lateral sclerosis, progressive supranuclear palsy, Refsum's disease, Sandhoff disease, Schilder's disease, spinal cord injury, spinal muscular atrophy (SMA), SteeleRichardson-Olszewski disease, and Tabes dorsalis.

In some embodiments, the iMNs as disclosed herein can be used in cellular models of human motor neuron disease, where such models could be used for basic research and drug discovery, e.g., to find treatments for motor neuron diseases or disorders including but not limited to: amyotrophic lateral sclerosis (ALS), also called Lou Gehrig's disease or classical motor neuron disease; progressive bulbar palsy, also called progressive bulbar atrophy; pseudobulbar palsy; primary lateral sclerosis (PLS); progressive muscular atrophy; spinal muscular atrophy (SMA, including SMA type 1, also called Werdnig-Hoffmann disease, SMA type II, and SMA type III, also called Kugelberg-Welander disease); Fazio-Londe disease; Kennedy disease, also known as progressive spinobulbar muscular atrophy; congenital SMA with arthrogryposis or post-polio syndrome (PPS).

In an exemplary embodiment, gene therapy can be used to insert DNA into a fibroblast which is transdifferentiated into a iN or iMN, where the fibroblast is from a patient or subject with a genetic defect or a defect of unknown origin in their neuron or motor neurons, followed by the transdifferentiation of the fibroblast into a iN or iMN. The thus formed iN or iMN population may then be used as a cellular model for the disorder associated with the genetic defect or any other abnormality carried by these cells. In some embodiments, the cellular model may be used for the development of drugs. In addition, a population of iNs or iMNs transdifferentiated from fibroblasts obtained from a subject with a neuron disease (e.g., neurodegenerative disorder or disease) or motor neuron disease may serve for drug development and testing for the specific patient from which they were developed in the course of personalized medicine.

In another exemplary embodiment neural stem cells, neural precursors or neural progenitors may be developed from any source of somatic cells, e.g., the gonads, bone marrow, brain biopsy or any transdifferentiation of somatic cells obtained from a patient with motor neuron disorder of any etiology, and directed to convert by transdifferentiation method as disclosed herein into a population of motor neurons. Such iMN population may then be used as a cellular model for the motor neuron disorder of the patient. The cellular model may be used for the development of drugs. In addition, the thus formed population may serve for drug development and testing for the specific patient from which they were developed in the course of personalized medicine.

In another exemplary embodiment neural stem cells, neural precursors or neural progenitors may be developed from any source of somatic cells, e.g., the gonads, bone marrow, brain biopsy or any transdifferentiation of somatic cells obtained from a patient with neurodegenerative disorder of any etiology, and directed to convert by transdifferentiation method as disclosed herein into a population of neurons. Such iN population may then be used as a cellular model for the neurodegenerative disorder of the patient. The cellular model may be used for the development of drugs. In addition, the thus formed population may serve for drug development and testing for the specific patient from which they were developed in the course of personalized medicine.

In some embodiments, an iN population as disclosed herein may serve for testing and high throughput screening of molecules for neurotoxic, teratogenic, neurotrophic, neuroprotective and neurodegenerative effects. In accordance with another embodiment, the iNs can be used for studying exogenous diseases and disorders of neurons. In one exemplary embodiment, the iNs can be used to study viral infections of neurons such as West Nile virus.

In some embodiments, an iMN population as disclosed herein may serve for testing and high throughput screening of molecules for neurotoxic, teratogenic, neurotrophic, neuroprotective and neurodegenerative effects. In accordance with another embodiment, the iMNs can be used for studying exogenous diseases and disorders of motor neurons. In one exemplary embodiment, the iMNs can be used to study viral infections of motor neurons such as polio.

In some embodiments, altering the surface antigens of the iNs or iMNs produced by the methods as disclosed herein can reduce the likelihood that iNs or iMNs will cause an immune response. The iNs or iMNs with altered surface antigens can then be administered to the subject. The cell surface antigens can be altered prior to, during, or after the fibroblasts are transdifferentiated into iNs or iMNs.

The subject of the invention can include individual humans, domesticated animals, livestock (e.g., cattle, horses, pigs, etc.), pets (like cats and dogs).

Accordingly, the methods for treatment as described herein can be combined with other methods of treating motor neuron diseases which are known by a skilled physician in the art of neurological treatment of motor neuron diseases.

Kits

The cells and components such as one or more ALK4, ALK5, and ALK7 inhibiting agents, and/or a PLK1 inhibiting agent, and/or one or more MN-inducing factors or agents can be provided in a kit. The kit includes (a) the cells and components described herein, e.g., a composition(s) that includes a cell and component(s) described herein, and, optionally (b) informational material. The informational material can be descriptive, instructional, marketing or other material that relates to the methods described herein and/or the use of a compound(s) described herein for the methods described herein.

The informational material of the kits is not limited in its form. In one embodiment, the informational material can include information about production of a cell, the nature of the components such as the transcription factor, concentration of components, date of expiration, batch or production site information, and so forth. In one embodiment, the informational material relates to methods for administering the cells or other components.

In one embodiment, the informational material can include instructions to administer a compound(s) component such as a ALK4, ALK5, and ALK7 inhibiting agent described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a component(s) described herein to a suitable subject, e.g., a human, e.g., a human having or at risk for a disorder described herein or to a cell in vitro.

In one embodiment, the informational material can include instructions to administer a compound(s) component such as a PLK1 inhibiting agent described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a component(s) described herein to a suitable subject, e.g., a human, e.g., a human having or at risk for a disorder described herein or to a cell in vitro.

In one embodiment, the informational material can include instructions to administer a compound(s) component such as a ALK4, ALK5, ALK7 inhibiting agent, and a PLK1 inhibiting agent described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a component(s) described herein to a suitable subject, e.g., a human, e.g., a human having or at risk for a disorder described herein or to a cell in vitro.

In one embodiment, the informational material can include instructions to administer a compound(s) component such as a ALK4, ALK5, and ALK7 inhibiting agent and/or a PLK1 inhibiting agent, together with a transcription factor described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a component(s) described herein to a suitable subject, e.g., a human, e.g., a human having or at risk for a disorder described herein or to a cell in vitro.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound(s) described herein, the composition of the kit can include other ingredients, such as a solvent or buffer, a stabilizer, a preservative, and/or an additional agent, e.g., for reprogramming a somatic cell (e.g., fibroblast) such as a somatic cell (e.g., in vitro or in vivo) or for treating a condition or disorder described herein. Alternatively, the other ingredients can be included in the kit, but in different compositions or containers than a component described herein. In such embodiments, the kit can include instructions for admixing a component(s) described herein and the other ingredients, or for using a component(s) described herein together with the other ingredients, e.g., instructions on combining the two agents prior to administration.

The kit can include one or more containers for the composition containing a component(s) described herein. In some embodiments, the kit contains separate containers (e.g., two separate containers for the two agents), dividers or compartments for the component(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a component described herein. The containers of the kits can be air tight, waterproof (e.g., impermeable to changes in moisture or evaporation), and/or light-tight.

The kit optionally includes a device suitable for administration of the component, e.g., a syringe, inhalant, pipette, forceps, measured spoon, dropper (e.g., eye dropper), swab (e.g., a cotton swab or wooden swab), or any such delivery device.

Pharmaceutical Compositions Comprising a Population of iNs or iMNs.

In another aspect of the invention, the methods provide use of an isolated population of iNs or iMNs as disclosed herein. In one embodiment of the invention, an isolated population of iNs as disclosed herein may be used for the production of a pharmaceutical composition, for the use in transplantation into subjects in need of treatment, e.g. a subject that has, or is at risk of developing a neurodegenerative disease or disorder, for example but not limited to subjects with Non-limiting examples of neurodegenerative disorders include polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, Guillain-Barré syndrome, ischemia stroke, Krabbe disease, kuru, Lewy body dementia, multiple sclerosis, multiple system atrophy, non-Huntingtonian type of Chorea, Parkinson's disease, Pelizaeus-Merzbacher disease, Pick's disease, primary lateral sclerosis, progressive supranuclear palsy, Refsum's disease, Sandhoff disease, Schilder's disease, spinal cord injury, spinal muscular atrophy (SMA), SteeleRichardson-Olszewski disease, and Tabes dorsalis.

In one embodiment, an isolated population of iNs may be genetically modified. In another aspect, the subject may have or be at risk of a motor neuron disease, e.g., carry a particular mutation for susceptibility for a neurodegenerative disorder which has not yet been observed or detected along with neurodegenerative disorder symptoms. In some embodiments, an isolated population of iNs as disclosed herein may be autologous and/or allogenic. In some embodiments, the subject is a mammal, and in other embodiments the mammal is a human.

In one embodiment of the invention, an isolated population of iMNs as disclosed herein may be used for the production of a pharmaceutical composition, for the use in transplantation into subjects in need of treatment, e.g. a subject that has, or is at risk of developing a motor neuron disease or disorder, for example but not limited to subjects with congenital and acquired ALS or SMA. In one embodiment, an isolated population of iMNs may be genetically modified. In another aspect, the subject may have or be at risk of a motor neuron disease, e.g., carry a particular mutation for susceptibility for ALS by has not yet observed or detected ALS symptoms. In some embodiments, an isolated population of iMNs as disclosed herein may be autologous and/or allogenic. In some embodiments, the subject is a mammal, and in other embodiments the mammal is a human.

The use of an isolated population of iNs or iMNs as disclosed herein provides advantages over existing methods because the iNs or iMNs can be reprogrammed from a somatic cell, e.g., fibroblast obtained or harvested from the subject administered an isolated population of iNs or iMNs. This is highly advantageous as it provides a renewable source of functional neurons or functional motor neurons, respectively, for transplantation into a subject, in particular a substantially pure population of iNs or iMNs that do not have the risks and limitations of iNs or iMNs derived from other systems, such as from iPS cells which have risks of formation of teratomas (Lafamme and Murry, 2005, Murry et al, 2005; Rubart and Field, 2006).

In another embodiment, an isolated population of iNs or iMNs can be used as models for studying properties of neurons or motor neurons, or pathways of development of a somatic cell, e.g., fibroblast into neuron cells or motor neuron cells, respectively. In some embodiments, the iNs or iMNs cells may be genetically engineered to comprise markers operatively linked to promoters that are expressed when a marker is expressed or secreted, for example, a marker can be operatively linked to Hb9 promoter, so that the marker is expressed when the cell becomes a functional motor neuron. In some embodiments, a population of iNs can be used as a model for studying the differentiation pathway of cells which differentiate into neurons. In some embodiments, a population of iMNs can be used as a model for studying the differentiation pathway of cells which differentiate into motor neurons. In other embodiments, the iNs may be used as models for studying the role of neurons in development and in the development of neurodegenerative diseases or disorders. In other embodiments, the iMNs may be used as models for studying the role of motor neurons in development and in the development of motor neuron disease or disorders. In some embodiments, the iMNs can be from a normal subject, or from a subject which carries a mutation and/or polymorphism (e.g. a mutation in the SOD1 gene is one form of the inherited form of ALS), as well as effect of mutations on late onset ALS, which can be use to identify small molecules and other therapeutic agents that can be used to treat subjects with ALS with such mutations or polymorphism in ALS associated genes. In some embodiments, the iMNs may be genetically engineered to correct the polymorphism in the SOD1 gene, or other ALS susceptibility genes, including but not limited to, heavy neurofilament chain (NFH), dynactin, vescicular binding protein 1 gene and the ALSIN (ALS2) gene, prior to being administered to a subject in the therapeutic treatment of a subject with ALS. In some embodiments, the iMNs may be genetically engineered to carry a mutation and/or polymorphism for studying the effects of the mutation and/or polymorphism on the development and contribution to the motor neuron disease.

In one embodiment of the invention relates to a method of treating a neurodegenerative disease or disorder, e.g., Alzheimer's disease, Parkinson's disease, or multiple sclerosis, in a subject comprising administering an effective amount of a composition comprising a population of iNs as disclosed herein to a subject with a neurodegenerative disease, e.g., AD, PD, or MS. In a further embodiment, the invention provides a method for treating a neurodegenerative disorder or disease, e.g., AD, PD, or MS, comprising administering a composition comprising a population of iNs as disclosed herein to a subject that has, or has increased risk of developing a neurodegenerative disorder or disease, e.g., AD, PD, or MS, in an effective amount sufficient to produce neurons which can support degenerating or dying neurons in the subject.

In one embodiment of the invention relates to a method of treating a motor neuron disease, e.g., ALS or SMA in a subject comprising administering an effective amount of a composition comprising a population of iMNs as disclosed herein to a subject with a motor neuron disease, e.g., ALS or SMA. In a further embodiment, the invention provides a method for treating a motor neuron disease, e.g., ALS or SMA, comprising administering a composition comprising a population of iMNs as disclosed herein to a subject that has, or has increased risk of developing a motor neuron disease, e.g., ALS or SMA, in an effective amount sufficient to produce motor neurons which can support degenerating or dying motor neurons in the subject.

In some embodiments, a population of iNs can be administered to a subject in combination with other treatment for neurodegenerative disorders or diseases, such as, for example, administration on combination with other agents or stem cells, e.g, embryonic stem cells used for the treatment of neurodegenerative disorders or diseases.

In some embodiments, a population of iMNs can be administered to a subject in combination with other treatment for motor neuron diseases, such as, for example, administration on combination with riluzole, RNA interference (RNAi) for ALS susceptibility or mutated genes (e.g., RNAi of mutant SOD1 genes, or RNAi for any of the mutant NFH, dynactin, vesicular binding protein or ALSIN genes), neurotrophic factors (e.g., IGF-1, EPO, CTNF, BDNF, VEGF), anti-oxidative agents such as HIF-loc, amino acids, e.g., creatine, as well as small molecules drugs such as ceftriaxone, lithium, xaliproden, pioglitazone, pyridostigmine and seligiline and other agents or stem cells, e.g, embryonic stem cells used for the treatment of motor neuron diseases.

In one embodiment of the above methods, the subject is a human and a population of iNs as disclosed herein are human cells.

In one embodiment of the above methods, the subject is a human and a population of iMNs as disclosed herein are human cells.

A population of iNs or iMNs as disclosed herein can be administered to any suitable location in the subject. In some embodiments, the invention contemplates that a population of iNs or iMNs as disclosed herein are administered directly to the spinal cord of a subject, or is administered systemically. In some embodiments, a population of iNs or iMNs as disclosed herein can be administered in a capsule in the blood vessel or any suitable site where administered population of iNs or iMNs can integrate into the spinal cord and send axonal projections which make synaptic contact with the muscle tissues in the subject.

The disclosure is also directed to a method of treating a subject with a motor neuron disease, e.g., ALS or SMA which occurs as a consequence of genetic defect, physical injury, environmental insult or conditioning, bad health, obesity and other a motor neuron disease risk factors commonly known by a person of ordinary skill in the art. Efficacy of treatment can be monitored by clinically accepted criteria and tests, which include for example, using Electromyography (EMG), which is used to diagnose muscle and nerve dysfunction and spinal cord disease, and measure the speed at which impulses travel along a particular nerve. EMG records the electrical activity from the brain and/or spinal cord to a peripheral nerve root (found in the arms and legs) that controls muscles during contraction and at rest. One can also monitor efficacy of treatment using a nerve conduction velocity study to measure electrical energy to test the nerve's ability to send a signal, as well as laboratory screening tests of blood, urine, as well as magnetic resonance imaging (MRI), which uses computer-generated radio waves and a powerful magnetic field to produce detailed images of body structures including tissues, organs, bones, and nerves to detect and monitor degenerative disorders. In some embodiments, efficacy of treatment can also be assessed by a muscle or nerve biopsy can help confirm nerve disease and nerve regeneration. A small sample of the muscle or nerve is removed under local anesthetic and studied under a microscope. The sample may be removed either surgically, through a slit made in the skin, or by needle biopsy, in which a thin hollow needle is inserted through the skin and into the muscle. A small piece of muscle remains in the hollow needle when it is removed from the body. In some embodiments, efficacy of treatment can also be monitored by a transcranial magnetic stimulation to study areas of the brain related to motor activity.

Other motor neuron diseases which can be treated by the methods as disclosed herein include, but are not limited to: Amyotrophic lateral sclerosis (ALS), Progressive bulbar palsy, Pseudobulbar palsy, Primary lateral sclerosis (PLS), Progressive muscular atrophy, Spinal muscular atrophy (SMA), including Type I (also called Werdnig-Hoffmann disease), Type II, Type III (Kugelberg-Welander disease), Fazio-Londe disease, Kennedy's disease also known as progressive spinobulbar muscular atrophy; congenital SMA with arthrogryposis, Post-polio syndrome (PPS) and traumatic spinal cord injury.

ALS, also called Lou Gehrig's disease or classical motor neuron disease, is a progressive, ultimately fatal disorder that eventually disrupts signals to all voluntary muscles. In the United States, doctors use the terms motor neuron disease and ALS interchangeably. Both upper and lower motor neurons are affected. Approximately 75 percent of people with classic ALS will also develop weakness and wasting of the bulbar muscles (muscles that control speech, swallowing, and chewing). Symptoms are usually noticed first in the arms and hands, legs, or swallowing muscles. Muscle weakness and atrophy occur disproportionately on both sides of the body. Affected individuals lose strength and the ability to move their arms, legs, and body. Other symptoms include spasticity, exaggerated reflexes, muscle cramps, fasciculations, and increased problems with swallowing and forming words. Speech can become slurred or nasal. When muscles of the diaphragm and chest wall fail to function properly, individuals lose the ability to breathe without mechanical support. Although the disease does not usually impair a person's mind or personality, several recent studies suggest that some people with ALS may have alterations in cognitive functions such as problems with decision-making and memory. ALS most commonly strikes people between 40 and 60 years of age, but younger and older people also can develop the disease. Men are affected more often than women. Most cases of ALS occur sporadically, and family members of those individuals are not considered to be at increased risk for developing the disease. (There is a familial form of ALS in adults, which often results from mutation of the superoxide dismutase gene, or SOD1, located on chromosome 21.) A rare juvenile-onset form of ALS is genetic. Most individuals with ALS die from respiratory failure, usually within 3 to 5 years from the onset of symptoms. However, about 10 percent of affected individuals survive for 10 or more years.

Progressive bulbar palsy, also called progressive bulbar atrophy, involves the bulb-shaped brain stem—the region that controls lower motor neurons needed for swallowing, speaking, chewing, and other functions. Symptoms include pharyngeal muscle weakness (involved with swallowing), weak jaw and facial muscles, progressive loss of speech, and tongue muscle atrophy. Limb weakness with both lower and upper motor neuron signs is almost always evident but less prominent. Affected persons have outbursts of laughing or crying (called emotional lability). Individuals eventually become unable to eat or speak and are at increased risk of choking and aspiration pneumonia, which is caused by the passage of liquids and food through the vocal folds and into the lower airways and lungs. Stroke and myasthenia gravis each have certain symptoms that are similar to those of progressive bulbar palsy and must be ruled out prior to diagnosing this disorder. In about 25 percent of ALS cases early symptoms begin with bulbar involvement. Some 75 percent of individuals with classic ALS eventually show some bulbar involvement. Many clinicians believe that progressive bulbar palsy by itself, without evidence of abnormalities in the arms or legs, is extremely rare.

Pseudobulbar palsy, which shares many symptoms of progressive bulbar palsy, is characterized by upper motor neuron degeneration and progressive loss of the ability to speak, chew, and swallow.

Progressive weakness in facial muscles leads to an expressionless face. Individuals may develop a gravelly voice and an increased gag reflex. The tongue may become immobile and unable to protrude from the mouth. Individuals may also experience emotional lability.

Primary lateral sclerosis (PLS) affects only upper motor neurons and is nearly twice as common in men as in women. Onset generally occurs after age 50. The cause of PLS is unknown. It occurs when specific nerve cells in the cerebral cortex (the thin layer of cells covering the brain which is responsible for most higher level mental functions) that control voluntary movement gradually degenerate, causing the muscles under their control to weaken. The syndrome—which scientists believe is only rarely hereditary—progresses gradually over years or decades, leading to stiffness and clumsiness of the affected muscles. The disorder usually affects the legs first, followed by the body trunk, arms and hands, and, finally, the bulbar muscles. Symptoms may include difficulty with balance, weakness and stiffness in the legs, clumsiness, spasticity in the legs which produces slowness and stiffness of movement, dragging of the feet (leading to an inability to walk), and facial involvement resulting in dysarthria (poorly articulated speech). Major differences between ALS and PLS (considered a variant of ALS) are the motor neurons involved and the rate of disease progression. PLS may be mistaken for spastic paraplegia, a hereditary disorder of the upper motor neurons that causes spasticity in the legs and usually starts in adolescence. Most neurologists follow the affected individual's clinical course for at least 3 years before making a diagnosis of PLS. The disorder is not fatal but may affect quality of life. PLS often develops into ALS.

Progressive muscular atrophy is marked by slow but progressive degeneration of only the lower motor neurons. It largely affects men, with onset earlier than in other MNDs. Weakness is typically seen first in the hands and then spreads into the lower body, where it can be severe. Other symptoms may include muscle wasting, clumsy hand movements, fasciculations, and muscle cramps. The trunk muscles and respiration may become affected. Exposure to cold can worsen symptoms. The disease develops into ALS in many instances.

Spinal muscular atrophy (SMA) is a hereditary disease affecting the lower motor neurons. Weakness and wasting of the skeletal muscles is caused by progressive degeneration of the anterior horn cells of the spinal cord. This weakness is often more severe in the legs than in the arms. SMA has various forms, with different ages of onset, patterns of inheritance, and severity and progression of symptoms. Some of the more common SMAs are described below.

SMA type I, also called Werdnig-Hojfmann disease, is evident by the time a child is 6 months old. Symptoms may include hypotonia (severely reduced muscle tone), diminished limb movements, lack of tendon reflexes, fasciculations, tremors, swallowing and feeding difficulties, and impaired breathing. Some children also develop scoliosis (curvature of the spine) or other skeletal abnormalities. Affected children never sit or stand and the vast majority usually die of respiratory failure before the age of 2. Symptoms of SMA type II usually begin after the child is 6 months of age. Features may include inability to stand or walk, respiratory problems, hypotonia, decreased or absent tendon reflexes, and fasciculations. These children may learn to sit but do not stand. Life expectancy varies, and some individuals live into adolescence or later. Symptoms of SMA type Ill (Kugelberg-Welander disease) appear between 2 and 17 years of age and include abnormal gait; difficulty running, climbing steps, or rising from a chair; and a fine tremor of the fingers. The lower extremities are most often affected. Complications include scoliosis and joint contractures—chronic shortening of muscles or tendons around joints, caused by abnormal muscle tone and weakness, which prevents the joints from moving freely.

Symptoms of Fazio-Londe disease appear between 1 and 12 years of age and may include facial weakness, dysphagia (difficulty swallowing), stridor (a high-pitched respiratory sound often associated with acute blockage of the larynx), difficulty speaking (dysarthria), and paralysis of the eye muscles. Most individuals with SMA type III die from breathing complications.

Kennedy disease, also known as progressive spinobulbar muscular atrophy, is an X-linked recessive disease. Daughters of individuals with Kennedy disease are carriers and have a 50 percent chance of having a son affected with the disease. Onset occurs between 15 and 60 years of age. Symptoms include weakness of the facial and tongue muscles, hand tremor, muscle cramps, dysphagia, dysarthria, and excessive development of male breasts and mammary glands. Weakness usually begins in the pelvis before spreading to the limbs. Some individuals develop noninsulin-dependent diabetes mellitus. The course of the disorder varies but is generally slowly progressive. Individuals tend to remain ambulatory until late in the disease. The life expectancy for individuals with Kennedy disease is usually normal. Congenital SMA with arthrogryposis (persistent contracture of joints with fixed abnormal posture of the limb) is a rare disorder. Manifestations include severe contractures, scoliosis, chest deformity, respiratory problems, unusually small jaws, and drooping of the upper eyelids.

Post-polio syndrome (PPS) is a condition that can strike polio survivors decades after their recovery from poliomyelitis. PPS is believed to occur when injury, illness (such as degenerative joint disease), weight gain, or the aging process damages or kills spinal cord motor neurons that remained functional after the initial polio attack. Many scientists believe PPS is latent weakness among muscles previously affected by poliomyelitis and not a new MND. Symptoms include fatigue, slowly progressive muscle weakness, muscle atrophy, fasciculations, cold intolerance, and muscle and joint pain. These symptoms appear most often among muscle groups affected by the initial disease. Other symptoms include skeletal deformities such as scoliosis and difficulty breathing, swallowing, or sleeping. Symptoms are more frequent among older people and those individuals most severely affected by the earlier disease. Some individuals experience only minor symptoms, while others develop SMA and, rarely, what appears to be, but is not, a form of ALS. PPS is not usually life threatening. Doctors estimate the incidence of PPS at about 25 to 50 percent of survivors of paralytic poliomyelitis.

In some embodiments, the effects of administration of a population of iNs or iMNs as disclosed herein to a subject in need thereof is associated with improved exercise tolerance or other quality of life measures, and decreased mortality. The effects of cellular therapy can be evident over the course of days to weeks after the procedure. However, beneficial effects may be observed as early as several hours after the procedure, and may persist for several years.

In some embodiments, the iNs or iMNs can be used for transplantation into any tissue of interest, where such tissues could be neural tissues (central nervous system or peripheral nervous system, e.g. spinal cord, nerve bundles, motor nerves, nerve ganglia) or non-neural tissues (muscle, liver, lungs). The iNs or iMNs can be transplanted into the spinal cord at any position from the cervical to lumbar regions. One of skill in the art can determine what procedures would be necessary for transplanting the cells into a particular position in the spinal cord, e.g., in some embodiments, a laminectomy may be appropriate to facility entry to the spinal cord, while in other embodiments the cells could be administered by directly accessing the spinal cord, as may be possible for neonatal applications, or administration to adult subjects by inserted the injection apparatus between vertebral bodies (similar to a spinal tap), to deliver the cells either into nervous tissue or intra thecal or into any other appropriate site.

In accordance with one aspect of the invention, when the iNs are used in a therapeutic application wherein the cells are expected to exhibit functions similar or identical to neuron functions. In accordance with one aspect of the invention, when the iMNs are used in a therapeutic application wherein the cells are expected to exhibit functions similar or identical to motor neuron functions. In one embodiment, the iNs or iMNs are transplanted using procedures to target the cells to selected sites. In an exemplary embodiment, when iNs or iMNs are introduced into the spinal cord, the cells may be targeted to spinal cord grey matter, including the dorsal or ventral horn of the grey matter. In another exemplary embodiment, iNs or iMNs can be targeted to other sites including, but not limited to, an emerging ventral or dorsal root, a dorsal root ganglion, a spinal nerve, a peripheral nerve a motor nerve, or any other appropriate site as determined by one of skill in the art. In one embodiment, the iNs or iMNs are transplanted directly or indirectly (e.g. ex vivo) to mammals, preferably, to humans.

In some other embodiments, the iNs or iMNs can be used as carriers for gene therapy, or as carriers for protein delivery.

In some embodiments, a population of iNs or iMNs as disclosed herein may be used for tissue reconstitution or regeneration in a human patient or other subject in need of such treatment. The cells are administered in a manner that permits them to graft or migrate to the intended tissue site and reconstitute or regenerate the functionally deficient area. Special devices are available that are adapted for administering cells capable of reconstituting a population of iNs or iMNs as disclosed herein into the spinal cord or at an alternative desired location. The cells may be administered to a recipient by injection, or administered by intramuscular injection.

To determine the suitability of cell compositions for therapeutic administration, the cells can first be tested in a suitable animal model. At one level, cells are assessed for their ability to survive and maintain their phenotype in vivo. Cell compositions can be administered to immunodeficient animals (such as nude mice, or animals rendered immunodeficient chemically or by irradiation). Tissues are harvested after a period of regrowth, and assessed as to whether the administered cells or progeny thereof are still present.

This can be performed by administering cells that express a detectable label (such as green fluorescent protein, or beta-galactosidase); that have been pre-labeled (for example, with BrdU or [3H]thymidine), or by subsequent detection of a constitutive cell marker (for example, using human-specific antibody). The presence and phenotype of the administered population of iNs or iMNs can be assessed by immunohistochemistry or ELISA using human-specific antibody, or by RT-PCR analysis using primers and hybridization conditions that cause amplification to be specific for human polynucleotides, according to published sequence data.

A number of animal models for testing in models of motor neuron diseases are available for such testing, and are commonly known in the art, for example as the S0D1(G93A) mutant mouse and SMA (B6.129-Smnl^{tmlJme J}) mouse models from Jackson laboratories.

In some embodiments, a population of iNs or iMNs as disclosed herein may be administered in any physiologically acceptable excipient, where the cells may find an appropriate site for regeneration and differentiation. In some embodiments, a population of iNs or iMNs as disclosed herein can be introduced by injection, catheter, or the like. In some embodiments, a population of iNs or iMNs as disclosed herein can be frozen at liquid nitrogen temperatures and stored for long periods of time, being capable of use on thawing. If frozen, a population of iNs or iMNs will usually be stored in a 10% DMSO, 50% FCS, 40% RPMI 1640 medium. Once thawed, the cells may be expanded by use of growth factors and/or feeder cells associated with culturing iNs or iMNs as disclosed herein.

In some embodiments, a population of iNs or iMNs as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising a population of iNs or iMNs as disclosed herein will be adapted in accordance with the route and device used for administration. In some embodiments, a composition comprising a population of iNs or iMNs can also comprise or be accompanied with one or more other ingredients that facilitate the engraftment or functional mobilization of the iNs or iMNs. Suitable ingredients include matrix proteins that support or promote adhesion of the iNs or iMNs, or complementary cell types, especially glial and/or muscle cells. In another embodiment, the composition may comprise resorbable or biodegradable matrix scaffolds.

In some embodiments, a population of iNs or iMNs as disclosed herein may be genetically altered in order to introduce genes useful in the iNs or iMNs, e.g. repair of a genetic defect in an individual, selectable marker, etc., or genes useful in selection against non-iNs or non-iMNs or for the selective suicide of implanted iNs or iMNs. In some embodiments, a population of iNs or iMNs can also be genetically modified to enhance survival, control proliferation, and the like. In some embodiments, a population of iNs or iMNs as disclosed herein can be genetically altering by transfection or transduction with a suitable vector, homologous recombination, or other appropriate technique, so that they express a gene of interest. In one embodiment, a iNs or iMNs is transfected with genes encoding a telomerase catalytic component (TERT), typically under a heterologous promoter that increases telomerase expression beyond what occurs under the endogenous promoter, (see International Patent Application WO 98/14592, which is incorporated herein by reference). In other embodiments, a selectable marker is introduced, to provide for greater purity of the population of iNs or iMNs. In some embodiments, a population of iNs or iMNs may be genetically altered using vector containing supernatants over a 8-16 h period, and then exchanged into growth medium for 1-2 days. Genetically altered iNs or iMNs can be selected using a drug selection agent such as puromycin, G418, or blasticidin, and then recultured.

Gene therapy can be used to either modify a cell to replace a gene product, to facilitate regeneration of tissue, to treat disease, or to improve survival of the cells following implantation into a subject (i.e. prevent rejection).

In an alternative embodiment, a population of iNs or iMNs as disclosed herein can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the differentiated cell type.

Many vectors useful for transferring exogenous genes into target iNs or iMNs as disclosed herein are available. The vectors may be episomal, e.g. plasmids, virus derived vectors such as cytomegalovirus, adenovirus, etc., or may be integrated into the target cell genome, through homologous recombination or random integration, e.g. retrovirus derived vectors such MMLV, HIV-1, ALV, etc. In some embodiments, combinations of retroviruses and an appropriate packaging cell line may also find use, where the capsid proteins will be functional for infecting the iNs or iMNs as disclosed herein. Usually, iNs or iMNs and virus will be incubated for at least about 24 hours in the culture medium. In some embodiments, iNs or iMNs are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The host cell specificity of the retrovirus is determined by the envelope protein, env (pi 20). The envelope protein is provided by the packaging cell line. Envelope proteins are of at least three types, ecotropic, amphotropic and xenotropic. Retroviruses packaged with ecotropic envelope protein, e.g. MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse. Amphotropic packaging cell lines include PA12 (Miller et al. (1985) Mol. Cell. Biol. 5:431-437); PA317 (Miller et al. (1986) Mol. Cell. Biol. 6:2895-2902) GRIP (Danos et al. (1988) PNAS 85:6460-6464). Retroviruses packaged with xenotropic envelope protein, e.g. AKR env, are capable of infecting most mammalian cell types, except murine cells. In some embodiments, the vectors may include genes that must later be removed, e.g. using a recombinase system such as Cre/Lox, or the cells that express them destroyed, e.g. by including genes that allow selective toxicity such as herpesvirus TK, Bcl-Xs, etc.

Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold, Various promoters are known that are induced in different cell types.

In one aspect of the disclosure, a population of iNs or iMNs as disclosed herein are suitable for administering systemically or to a target anatomical site. A population of iNs or iMNs can be grafted into or nearby a subject's spinal cord, for example, or may be administered systemically, such as, but not limited to, intraarterial or intravenous administration. In alternative embodiments, a population of iNs or iMNs of the disclosure can be administered in various ways as would be appropriate to implant in the central nervous system or peripheral nervous system, including but not limited to parenteral, including intravenous and intraarterial administration, intrathecal administration, intraventricular administration, intraparenchymal, intracranial, intracisternal, intrastriatal, and intranigral administration. Optionally, a population of iMNs can be administered in conjunction with an immunosuppressive agent.

In some embodiments, a population of iNs or iMNs can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art. A population of iNs or iMNs can be administered to a subject the following locations: clinic, clinical office, emergency department, hospital ward, intensive care unit, operating room, catheterization suites, and radiologic suites.

In other embodiments, a population of iNs or iMNs is stored for later implantation/infusion. A population of iNs or iMNs may be divided into more than one aliquot or unit such that part of a population of iNs or iMNs is retained for later application while part is applied immediately to the subject. Moderate to long-term storage of all or part of the cells in a cell bank is also within the scope of this invention, as disclosed in U.S. Patent Application Serial No. 20030054331 and Patent Application No. WO03024215, and is incorporated by reference in their entireties. At the end of processing, the concentrated cells may be loaded into a delivery device, such as a syringe, for placement into the recipient by any means known to one of ordinary skill in the art.

In some embodiments, a population of iNs or iMNs can be applied alone or in combination with other cells, tissue, tissue fragments, growth factors such as VEGF and other known angiogenic or arteriogenic growth factors, biologically active or inert compounds, resorbable plastic scaffolds, or other additive intended to enhance the delivery, efficacy, tolerability, or function of the population. In some embodiments, a population of iNs or iMNs may also be modified by insertion of DNA or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998).

In another aspect, in some embodiments, a population of iNs or iMNs could be combined with a gene encoding pro-angiogenic growth factor(s). Genes encoding anti-apoptotic factors or agents could also be applied. Addition of the gene (or combination of genes) could be by any technology known in the art including but not limited to adenoviral transduction, “gene guns,” liposome-mediated transduction, and retrovirus or lentivirus-mediated transduction, plasmid adeno-associated virus. Cells could be implanted along with a carrier material bearing gene delivery vehicle capable of releasing and/or presenting genes to the cells over time such that transduction can continue or be initiated. Particularly when the cells and/or tissue containing the cells are administered to a patient other than the patient from whom the cells and/or tissue were obtained, one or more immunosuppressive agents may be administered to the patient receiving the cells and/or tissue to reduce, and preferably prevent, rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell co-stimulation pathways, such as agents that interfere with the coupling of T-cells and B-cells via the CTLA4 and B7 pathways, as disclosed in U.S. Patent Pub. No 2002/0182211, which is incorporated herein by reference. In one embodiment, an immunosuppressive agent is cyclosporine A. Other examples include myophenylate mofetil, rapamicin, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the iMNs of the invention.

Pharmaceutical compositions comprising effective amounts of a population of iNs or iMNs are also contemplated by the disclosure. These compositions comprise an effective number iNs or iMNs, optionally, in combination with a pharmaceutically acceptable carrier, additive or excipient. In certain aspects of the disclosure, a population of iNs or iMNs can be administered to the subject in need of a transplant in sterile saline. In other aspects of the disclosure, a population of iNs or iMNs can be administered in Hanks Balanced Salt Solution (HBSS) or Isolyte S, pH 7.4. Other approaches may also be used, including the use of serum free cellular media. In one embodiment, a population of iNs or iMNs can be administered in plasma or fetal bovine serum, and DMSO. Systemic administration of a population of iNs or iMNs to the subject may be preferred in certain indications, whereas direct administration at the site of or in proximity to the diseased and/or damaged tissue may be preferred in other indications.

In some embodiments, a population of iNs or iMNs can optionally be packaged in a suitable container with written instructions for a desired purpose, such as the reconstitution or thawing (if frozen) of a population of iNs or iMNs prior to administration to a subject.

In one embodiment, an isolated population of iNs or iMNs as disclosed herein can be administered with a differentiation agent. In one embodiment, iNs or iMNs can be combined with the differentiation agent to administration into the subject. In another embodiment, the cells are administered separately to the subject from the differentiation agent. Optionally, if the cells are administered separately from the differentiation agent, there is a temporal separation in the administration of the iNs or iMNs and the differentiation agent. The temporal separation may range from about less than a minute in time, to about hours or days in time. The determination of the optimal timing and order of administration is readily and routinely determined by one of ordinary skill in the art.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the disclosure. Further, all patents, patent applications, and publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The following Examples are provided to illustrate the disclosure, and should not be construed as limiting thereof.

### EXAMPLES

The mammalian nervous system comprises many distinct neuronal subtypes, each with its own phenotype and differential sensitivity to degenerative disease. Although specific neuronal types can be isolated from rodents or engineered from stem cells for translational studies, transcription factor mediated reprogramming might provide a more direct route to their generation. Recent studies have demonstrated that the forced expression of select transcription factors is sufficient to convert mouse and human fibroblasts and stem cells directly into a variety of neuronal subtypes. However, the utility of this approach is currently limited by the low efficiency of conversion.

One potential solution is to identify small molecules that increase induced neuron generation. Such chemicals would enable the generation of large numbers of patient-specific neurons for disease studies and provide insight into the mechanisms that regulate neuronal induction by defined factors.

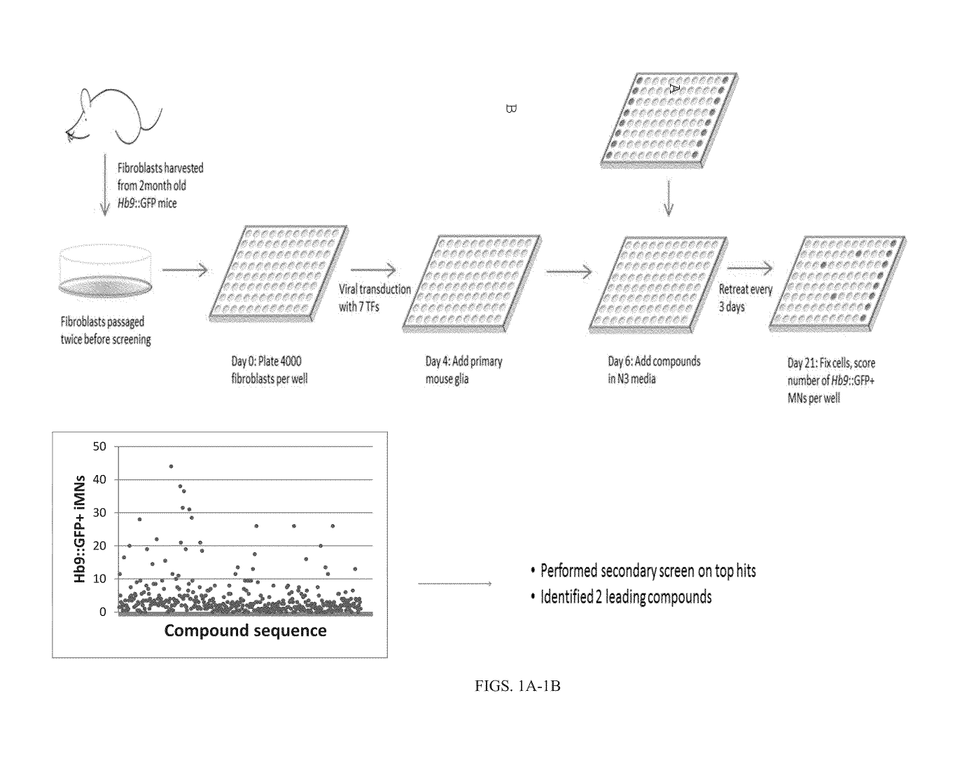

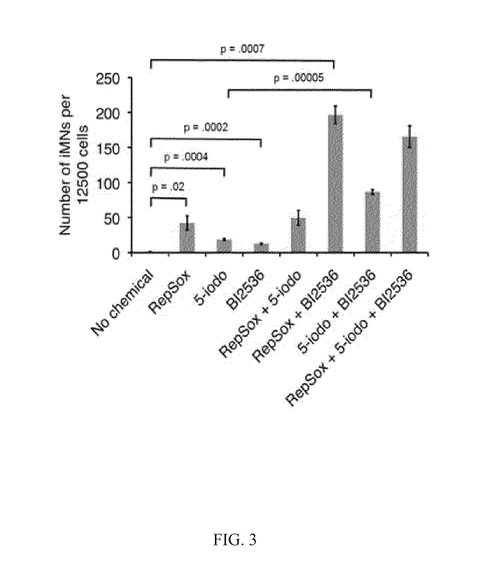

To this end, a functional reprogramming screen was used to identify small molecules that increase the rate of transcription factor-mediated conversion of mouse adult fibroblasts into Hb9::GFP+ spinal motor neurons (FIGS. 1A and 1B). An inhibitor of Activin-like kinases 4/5/7 (FIG. 2A) and a Polo-like kinase I (PLK1) inhibitor (FIG. 2B) each increased induced motor neuron formation by 5-10-fold (FIG. 3). In combination, the chemicals increased the rate of induced motor neuron formation by 50-fold (FIG. 3).

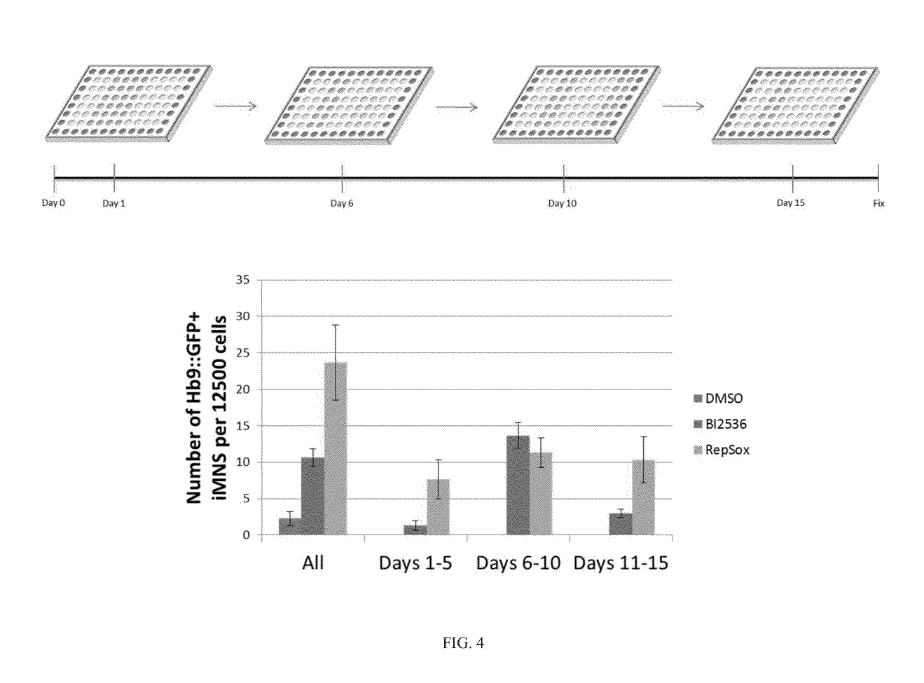

After using peptide or small molecule analogues Activin signaling was confirmed to be the functional target of one of these molecules during motor neuron induction, pulse treatments were performed at different times during reprogramming to determine when they are most effective. The Activin inhibitor was found to be effective when administered from days 1-5, 6-10, or 11-15 (FIG. 4). In contrast, the PLK1 inhibitor was only active during days 6-10 (FIG. 4), suggesting it acts on transient intermediates in the cultures. These results suggest that these chemicals act by divergent mechanisms.

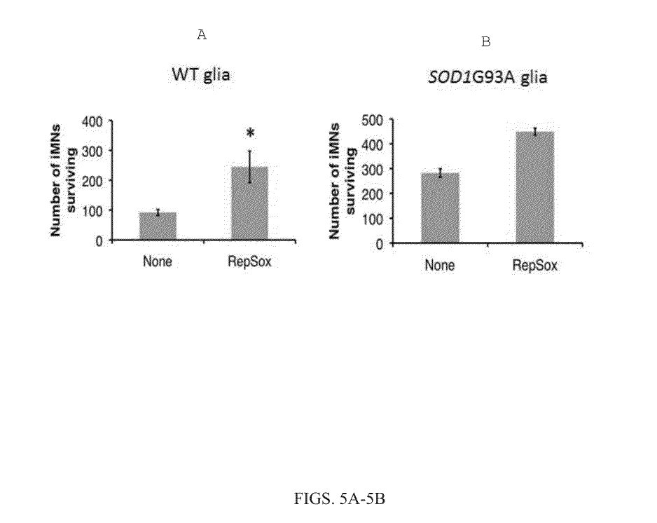

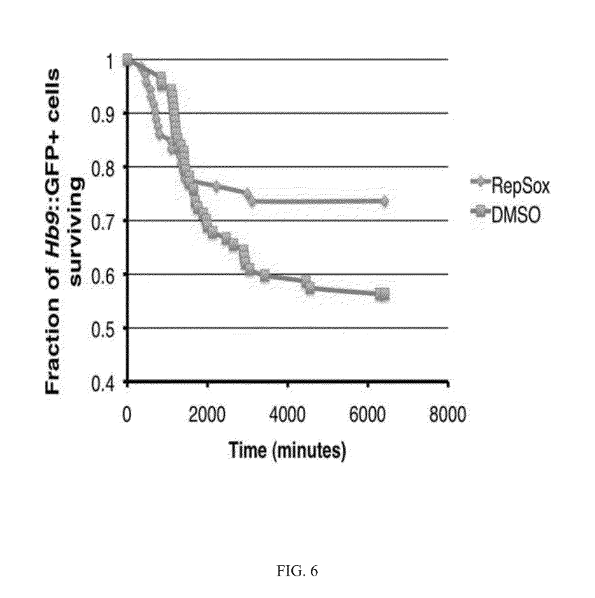

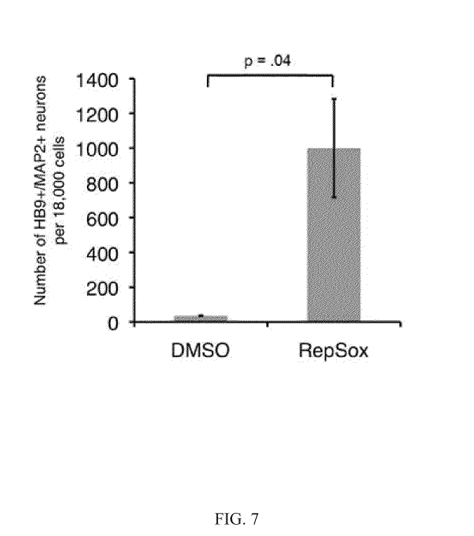

Because Activin inhibition was effective even after many motor neurons had appeared, the inventors hypothesized that it might enhance motor neuron survival. Indeed, chemical treatment greatly promoted the survival of flow-purified mouse and human motor neurons in culture, indicating that Activin inhibition can act by promoting neuronal survival (FIG. 5). Activin signaling also stimulated the survival of early reprogramming intermediates (FIG. 6). Both small molecules also increased the rate of conversion of human fibroblasts and embryonic stem cells into motor neurons, indicating that these chemicals should enable the generation of human patient-specific motor neurons for disease modeling (FIG. 7).

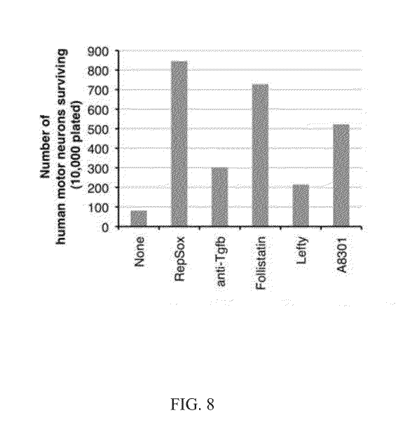

To determine if Activin inhibition increases conversion into other neuronal types, fibroblasts were transduced with Ascl1, Myt1l, and Brn2, transcription factors that induce the formation of non-motor neurons, and cultured the cells with or without the Activin inhibitor. Chemical treatment increased the number of neurons generated by 10-fold, indicating this approach may be applicable to a variety of neuronal types (FIG. 8).

The inventors have identified small molecules that increase the rate of direct conversion of mouse and human fibroblasts and stem cells into motor neurons. These results identify the Activin and the Polo-like kinase I signaling pathways as major roadblocks to induced neuron formation and indicate that many neurons are lost shortly after conversion. Finally, these chemicals should enable the efficient generation of induced neurons for patient-specific disease modeling.

In addition, it is likely that these compounds improve ES cell derived neuronal induction as well as direct conversion into alternate cell types including, for example, other neuronal subtypes and iPS cells

What is claimed is:

1. A method for improving the efficiency of neuron generation from a somatic cell, comprising (a) exposing the somatic cell to conditions sufficient for transdifferentiation of the somatic cell into a neuron; and (b) inhibiting one or both of Activin signaling and PLK1 signaling in the cell, thereby increasing the efficiency of neuron formation as compared with the efficiency when neither Activin signaling nor PLK1 signaling is inhibited.

2. A method for improving the efficiency of neuron generation from a less differentiated cell, comprising (a) exposing the less differentiated cell to conditions sufficient for differentiation of the less differentiated cell into a neuron; and (b) inhibiting one or both of Activin signaling and PLK1 signaling in the cell, thereby increasing the efficiency of neuron formation as compared with the efficiency when neither Activin signaling nor PLK1 signaling is inhibited.

3. A method according to any of claims 1-2, wherein the neuron is a motor neuron.

4. A method according to claim 1, wherein the somatic cell is a mouse cell.

5. A method according to claim 1, wherein the somatic cell is a human cell.

6. A method according to claim 1, wherein the somatic cell is a patient-derived cell.

7. A method according to claim 1, wherein the somatic cell is a fibroblast.

8. A method according to claim 1, wherein the conditions sufficient for transdifferentiation of the somatic cell are conditions sufficient for factor-mediated transdifferentiation.

9. A method according to claim 2, wherein the conditions sufficient for differentiation of the less differentiated cell are conditions sufficient for factor-mediated differentiation.

10. A method according to any of claims 1-9, wherein inhibiting Activin signaling comprises inhibiting Activin.

11. A method according to any of claims 1-10, wherein inhibiting Activin signaling comprises decreasing the level or activity of one or more of activin-like kinase 4 (ALK4), activin-like kinase 5 (ALK5), and activin-like kinase 7 (ALK7).

12. A method according to any of claims 1-11, wherein inhibiting PLK1 signaling comprises decreasing the level or activity of PLK1.

13. A method according to any of claims 1-12, wherein the neuron exhibits at least two characteristics of a functional neuron.

14. A method according to claim 13, wherein the neuron is a motor neuron and wherein the motor neuron exhibits at least two characteristics of a functional motor neuron.

15. A method according to any of claims 1-13, wherein the efficiency of neuron formation is increased at least 5-fold as compared with the efficiency when neither Activin signaling nor PLK1 signaling is inhibited.

16. A method according to claim 14, wherein a characteristic of the functional motor neuron is expression of at least two motor neuron specific genes selected from the group consisting of: β2-tubulins, Map2, synapsins, synaptophysin, synaptotagmins, NeuroD, Isl1, cholineacetyltransferase (ChAT).

17. A method according to claim 16, wherein the β2-tubulin is selected from Tubb2a and Tubb2b.

18. A method according to claim 16, wherein the synapsins is selected from Syn1 and Syn2.

19. A method according to claim 16, wherein the synaptotagmins are selected from: Syt1, Syt4, Syt13, Syt 16.

20. A method according to claim 16, wherein the ChAT is vesicular ChAT.

21. A method according to claim 13, wherein a characteristic of the functional neuron is expression of a decreased level of a fibroblast gene selected from the group of: Snail 1, thy1 and Fsp1, by a statistically significant level as compared to a somatic cell from which the neuron was derived.

22. A method according to claim 14, wherein a characteristic of the functional motor neuron is a functional characteristic selected from the group consisting of: ability to fire action potentials, produce an outward current in response to glycine, GABA or kainate, or produce an inward current in response to glutamate.

23. A method according to any of claims 1-22, wherein inhibiting Activin signaling comprises contacting the cell with an agent which decreases the level or activity of Activin.

24. A method according to claim 23, wherein the agent is selected from the group consisting of small organic or inorganic molecules; saccharines; oligosaccharides; polysaccharides; a biological macromolecule selected from the group consisting of antibodies, peptides, proteins, peptide analogs and derivatives, and dominant negative variants; peptidomimetics; nucleic acids selected from the group consisting of microRNAs, siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

25. A method according to claim 23 or 24, wherein the agent is RepSox or an analog or derivative thereof.

26. A method according to any of claims 1-22, wherein inhibiting PLK1 signaling comprises contacting the cell with an agent which decreases the level or activity of PLK1.

27. A method according to claim 26, wherein the agent is selected from the group consisting of small organic or inorganic molecules; saccharines; oligosaccharides; polysaccharides; a biological macromolecule selected from the group consisting of antibodies, peptides, proteins, peptide analogs and derivatives, and dominant negative variants; peptidomimetics; nucleic acids selected from the group consisting of microRNAs, siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

28. A method according to claim 26 or 27, wherein the agent is BI 2536 or an analog or derivative thereof.

29. A method according to any of claims 1-2, wherein the cell is obtained from a human subject.

30. A method according to claim 29, wherein the subject has, or is at risk of developing, a motor neuron disease or disorder.

31. A method according to claim 30, wherein the motor neuron disease or disorder is selected from the group consisting of amyotrophic lateral sclerosis (ALS) or spinal muscular atrophy (SMA) or a disease, condition, or symptom associated therewith.

32. An isolated population of neurons obtained by any of the methods of claims 1-31.

33. Use of an isolated population of neurons according to claim 32 for administering to a subject in need thereof.

34. A method for increasing neuron survival, comprising inhibiting Activin signaling in the cell, thereby increasing neuron survival compared to survival when Activin signaling is not inhibited.

35. A method for improving the survival of intermediates in a cell differentiation pathway, comprising inhibiting PLK1 signaling, thereby increasing the survival of intermediates in a cell differentiation pathway compared to survival when PLK1 signaling is not inhibited.
